SEQUENCE LISTING (38 sequences)

SEQ ID NO: 1 - DNA, 725 bp, Homo sapiens
  SHONa
tgttggtctagccttgcctcccgcatacccagtctagagcctggccacatgcccatcaaaagactctcactgctctgcctgccttcttctgtcctggcgtctataccctcagaccagccttgcatacctactcctgccagcctcctccaggaagccttgcctcctcaactctcctgcaccatcctcacatttcacctggccactgtcactgccagaggaggcaccactgacctgcctcatggcccagccactgcacccatctccaaccagacccttggtgtgttccccacccaatccatcacaagccactttcaagctttagggaagtgaaaagtactgcctgccctcaaaaaaaaaaaaaaaagaaaaagaaaagaaaggaaaagaaaagaaaacccagccgtaataaggagaccttatcaggagtactttacagtttataaagcatgtttctagccatcattgggaccacacggtggtcagtcattatcattttcattttatggatgaggacatcgtgactcaaagagcttggtaacgtgcccagggtcatcccacgggtgagtggaagggccaggattcaaaaccaggactccaggttccaagtgtcaggcccgttcgaccacactgtcctcccaccagccaagagggcaattctggaatcagccaggagggctgtcaatcacaggacaaataaaatagtctgtttttacttagctggaaaa

SEQ ID NO: 2 - protein, 93 aa, Homo sapiens
  SHON-alpha protein
MPIKRLSLLCLPSSVLASIPSDQPCIPTPASLLQEALPPQLSCTILTFHLATVTARGGTTDLPHGPATAPISNQTLGVFPTQSITSHFQALGK

SEQ ID NO: 3 - DNA, 893 bp, Homo sapiens
  SHONb
ccggggcagccgagggcccctgactcggctcctcgcggcgacatggatcggatggccagctccatgaagcaggtgcccaacccactgcccaaggtgctgagccggcgcggggtcggcgctgggctggaggcggcggagcgcgagagcttcgagcggactcagggtcgctgttggtctagccttgcctcccgcatacccagtctagagcctggccacatgcccatcaaaagactctcactgctctgcctgccttcttctgtcctggcgtctataccctcagaccagccttgcatacctactcctgccagcctcctccaggaagccttgcctcctcaactctcctgcaccatcctcacatttcacctggccactgtcactgccagaggaggcaccactgacctgcctcatggcccagccactgcacccatctccaaccagacccttggtgtgttccccacccaatccatcacaagccactttcaagctttagggaagtgaaaagtactgcctgccctcaaaaaaaaaaaaaaaagaaaaagaaaagaaaggaaaagaaaagaaaacccagccgtaataaggagaccttatcaggagtactttacagtttataaagcatgtttctagccatcattgggaccacacggtggtcagtcattatcattttcattttatggatgaggacatcgtgactcaaagagcttggtaacgtgcccagggtcatcccacgggtgagtggaagggccaggattcaaaaccaggactccaggttccaagtgtcaggcccgttcgaccacactgtcctcccaccagccaagagggcaattctggaatcagccaggagggctgtcaatcacaggacaaataaaatagtctgtttttacttagctggaaaa

SEQ ID NO: 4 - DNA, 775 bp, Homo sapiens
  SHONc
ccggggcagccgagggcccctgactcggctcctcgcggcgacatggatcggatggccagctccatgaagcaggtgcccaacccactgcccaaggtgctgagccggcgcggggtcggcgctgggctggaggcggcggagcgcgagagcttcgagcggactcagaccagccttgcatacctactcctgccagcctcctccaggaagccttgcctcctcaactctcctgcaccatcctcacatttcacctggccactgtcactgccagaggaggcaccactgacctgcctcatggcccagccactgcacccatctccaaccagacccttggtgtgttccccacccaatccatcacaagccactttcaagctttagggaagtgaaaagtactgcctgccctcaaaaaaaaaaaaaaaagaaaaagaaaagaaaggaaaagaaaagaaaacccagccgtaataaggagaccttatcaggagtactttacagtttataaagcatgtttctagccatcattgggaccacacggtggtcagtcattatcattttcattttatggatgaggacatcgtgactcaaagagcttggtaacgtgcccagggtcatcccacgggtgagtggaagggccaggattcaaaaccaggactccaggttccaagtgtcaggcccgttcgaccacactgtcctcccaccagccaagagggcaattctggaatcagccaggagggctgtcaatcacaggacaaataaaatagtctgtttttacttagctggaaaa

SEQ ID NO: 5 - protein, 151 aa, Homo sapiens
  SHON-beta protein
MDRMASSMKQVPNPLPKVLSRRGVGAGLEAAERESFERTQGRCWSSLASRIPSLEPGHMPIKRLSLLCLPSSVLASIPSDQPCIPTPASLLQEALPPQLSCTILTFHLATVTARGGTTDLPHGPATAPISNQTLGVFPTQSITSHFQALGK

SEQ ID NO: 6 - protein, 109 aa, Homo sapiens
  SHON-gamma protein
MDRMASSMKQVPNPLPKVLSRRGVGAGLEAAERESFERTQTSLAYLLLPASSRKPCLLNSPAPSSHFTWPLSLPEEAPLTCLMAQPLHPSPTRPLVCSPPNPSQATFKL

SEQ ID NO: 7 - DNA, 1140 bp, Homo sapiens
  Promoter 2 for SHONb and SHONc
taagcatcacacttctctctgcttgctggtaccctggctgacgtctgagcaggatgggacgcgcggctcagagcctaagataaaccgaaagtgtctacagggctttctaccaacaaataagctctgacgggttcctttgatgaggtttgggaggcaggtcccgggaggtgcccgcggactcagccccgaactccgggacctgggctctgcgatccttgcagagggcatgggcaggggcagaatggagaagtgcaggacggggaagcctgggagctgggtgggggcggccacccattgagccggatccggccaggcgcccacgctgcgccccgcccgcggggctgcgctttcccgggctgcggccgcagggctggtcagtccggcgctcccgggtcccaggcccggaaggagctaacgggctattcgcaggcgggctgggattcccccgggggaggccccactgcccggcccgcgtcatccccgcccatctccacgggccgtcgcgggatagccccctgcaggagcgggcagggtagtgggcggcgcttggcggagggcagcacgctcggggcgcgcggggactgcggccgaggggaggagagggcggtgcgcgggcgggggcggggcgagcgagggcatatcagggcagcccgcccaggccacgccccatccaggcctcgccccgtccgcaccccgccccggccctgcctagaacggcgggaccacggtgactgacaggactccagaccagtcaccacgcgcctcgaggccccgagaggctagtcggggccgagccagcggaggggctcctgaaggggcgggggcgggcggggaagccgttcggcgaggggcggggtctctggaagactggcagaactcacagccaatggcaggcgggagccgtcccgttagcgccggatccccgcgggtagggcggggcgggcggcgccgtggggatcccggggcagccgagggcccctgactcggctcctcgcggcgacatggatcggatggccagctccatgaagcaggtgcccaacccactgcccaaggtgctgagccggcgcggggtcggcgctgggctggaggcggcggagcgcgagagcttcgagcggactcaggtgaggacgggggaccccg

SEQ ID NO: 8 - DNA, 4013 bp, Homo sapiens
  Promoter 1 for SHONa
  modified_base (24)..(3983): a, c, t, g, unknown or other
gggcatccctggggcaccctcctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncctgggccacaggatatattaataattact

SEQ ID NO: 9 - DNA, 18 bp, Artificial Sequence
  Description of Artificial Sequence Synthetic primer
atcatatcgccgcgctcg

SEQ ID NO: 10 - DNA, 19 bp, Artificial Sequence
  Description of Artificial Sequence Synthetic primer
cgctcggtgaggatcttca

SEQ ID NO: 11 - protein, 6 aa, Artificial Sequence
  Description of Artificial Sequence Synthetic 6xHis tag
HHHHHH

SEQ ID NO: 12 - protein, 13 aa, Artificial Sequence
  Description of Artificial Sequence Synthetic peptide
YALEQKLISEEDL

SEQ ID NO: 13 - DNA, 21 bp, Artificial Sequence
  Description of Artificial Sequence Synthetic oligonucleotide
aatccatcacaagccactttc

SEQ ID NO: 14 - DNA, 23 bp, Artificial Sequence
  Description of Artificial Sequence Synthetic primer
acttccctaaagcttgaaagtgg

SEQ ID NO: 15 - DNA, 37 bp, Artificial Sequence
  Description of Artificial Sequence Synthetic primer
ccggacgcgtcgactagtacttttttttttttttttt

SEQ ID NO: 16 - DNA, 21 bp, Artificial Sequence
  Description of Artificial Sequence Synthetic primer
gtgatggattgggtggggaac

SEQ ID NO: 17 - DNA, 21 bp, Artificial Sequence
  Description of Artificial Sequence Synthetic primer
acaccaagggtctggttggag

SEQ ID NO: 18 - DNA, 23 bp, Artificial Sequence
  Description of Artificial Sequence Synthetic primer
atgcccatcaaaagactctcact

SEQ ID NO: 19 - DNA, 22 bp, Artificial Sequence
  Description of Artificial Sequence Synthetic primer
atggatcggatggccagctcca

SEQ ID NO: 20 - DNA, 17 bp, Artificial Sequence
  Description of Artificial Sequence Synthetic primer
ttcctgggcatggagtc

SEQ ID NO: 21 - DNA, 18 bp, Artificial Sequence
  Description of Artificial Sequence Synthetic primer
caggtctttgcggatgtc

SEQ ID NO: 22 - protein, 21 aa, Homo sapiens
MPIKRLSLLCLPSSVLASIPS

SEQ ID NO: 23 - protein, 4 aa, Homo sapiens
NQTL

SEQ ID NO: 24 - protein, 4 aa, Homo sapiens
KRLS

SEQ ID NO: 25 - protein, 6 aa, Homo sapiens
GVFPTQ

SEQ ID NO: 26 - protein, 6 aa, Homo sapiens
GVGAGL

SEQ ID NO: 27 - protein, 4 aa, Homo sapiens
SLPE

SEQ ID NO: 28 - DNA, 15 bp, Artificial Sequence
  Description of Artificial Sequence Synthetic oligonucleotide
ttataagaaaacaag

SEQ ID NO: 29 - DNA, 15 bp, Artificial Sequence
  Description of Artificial Sequence Synthetic oligonucleotide
cttgttttcttataa

SEQ ID NO: 30 - DNA, 14 bp, Artificial Sequence
  Description of Artificial Sequence Synthetic oligonucleotide
ctataattacttgt

SEQ ID NO: 31 - protein, 6 aa, Homo sapiens
KVLSRR

SEQ ID NO: 32 - protein, 6 aa, Homo sapiens
RESFER

SEQ ID NO: 33 - protein, 10 aa, Homo sapiens
GGTTDLPHGP

SEQ ID NO: 34 - protein, 10 aa, Homo sapiens
PATAPISNQT

SEQ ID NO: 35 - protein, 10 aa, Homo sapiens
NQTLGVFPTQ

SEQ ID NO: 36 - protein, 10 aa, Homo sapiens
PTQSITSHFQ

SEQ ID NO: 37 - DNA, 12 bp, Artificial Sequence
  Description of Artificial Sequence Synthetic oligonucleotide
cacagccaatgg

SEQ ID NO: 38 - DNA, 1392 bp, Homo sapiens
  Promoter 1 for SHONa
ttatttatttttatgtaagagatggtctcgctctattgtccagtctggagtgcagtggcaccatcatagctcagttttgcctgggactcctgggctcaagtgatcctcctgccttagcctcctgagtgctgggactacaggagcatgccaccatgcttagctgatctttaaaatttatttatttatttatttatttatttatttatttatttatttgagacagagtctcactctgttgctcaggctggagtgcagtggcgccatctcggttcactacaaccttcgcctcctgggttcaagtgattctcctgcctcggcctctcgagtagctgggattacaggcgagcgccaccacgcccggctaattttttgtatttttagtagagacagggtctcacttgttgaccaggctggcctcgaactcctgacctcaggtgagccgcctgcctcagcctcccaaagtgctgggattacaggtataagccatcgttcctggcctagaattttttttgtagagatggggtcttgctgtgttgcccaggctggtcttgaactcctgggctcaagcagtcctcccgccttggcttcccaaagtgttgggattacaggtgtgagccgccgtgcccggcctataattacttgttttcttataatgctccagggtcgctgttggtctagccttgcctcccgcatacccagtctagagcctggccacatgcccatcaaaagactctcactgctctgcctgccttcttctgtcctggcgtctataccctcagaccagccttgcatacctactcctgccagcctcctccaggaagccttgcctcctcaactctcctgcaccatcctcacatttcacctggccactgtcactgccagaggaggcaccactgacctgcctcatggcccagccactgcacccatctccaaccagacccttggtgtgttccccacccaatccatcacaagccactttcaagctttagggaagtgaaaagtactgcctgccctcaaaaaaaaaaaaaaaagaaaaagaaaagaaaggaaaagaaaagaaaacccagccgtaataaggagaccttatcaggagtactttacagtttataaagcatgtttctagccatcattgggaccacacggtggtcagtcattatcattttcattttatggatgaggacatcgtgactcaaagagcttggtaacgtgcccagggtcatcccacgggtgagtggaagggccaggattcaaaaccaggactccaggttccaagtgtcaggcccgttcgaccacactgtcctcccaccagccaagagggcaattctggaatcagccaggagggctgtcaatcacaggacaaataaaatagtctgtttttacttagctggaaaa

(19) United States
(12) Patent Application Publication
(10) Pub. No.: US 2021/0018504 A1
(43) Pub. Date: Jan. 21, 2021

(54) SHON AS A PROGNOSTIC BIOMARKER FOR CANCER AND AS A PREDICTOR OF RESPONSE TO ENDOCRINE THERAPY

(71) Applicant: Runlin Z. MA, Beijing (CN)

(72) Inventor: Dong-Xu LIU, Auckland (NZ)

(21) Appl. No.: 16/727,777

(22) Filed: Dec. 26, 2019

Related U.S. Application Data
(63) Continuation of application No. 15/103,581, filed on Jun. 10, 2016, now abandoned.

(30) Foreign Application Priority Data
Oct. 17, 2012 (NZ) 603056

(51) Int. Cl. G01N 33/574; C12Q 1/6886; A61K 31/138; A61K 31/4535; C07K 16/32

(52) U.S. Cl. CPC G01N 33/57415; C12Q 1/6886; A61K 31/138; A61K 31/4535; C07K 16/32; C12Q 2600/118; C12Q 2600/106; C12Q 2600/158; G01N 2333/723; G01N 2333/52; G01N 33/5748

(57) ABSTRACT

The estrogen-regulated gene sequence SHON has been discovered and isolated, and found to be a novel oncogene in mammary carcinoma and significantly associated with estrogen and progesterone receptor expression in breast cancer. The invention encompasses methods for predicting the responsiveness to endocrine therapy for breast cancer and providing a prognosis for disease-free and/or distant metastasis-free survival of a cancer patient.

### CROSS REFERENCE TO RELATED APPLICATIONS

This application is a continuation of U.S. application Ser. No. 15/103,581, filed on Jun. 10, 2016, which, in turn is a § 371 National State Application of PCT/NZ2013/000188, filed Oct. 17, 2013, which claims priority to NZ603056, filed Oct. 17, 2012, all of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Sep. 27, 2016, is named 2935720-17_SL.txt and is 21,657 bytes in size.

### FIELD OF THE INVENTION

The present invention relates to the fields of biotechnology, cancer cell biology and molecular medicine. Specifically, the present invention is directed to the prediction of the outcome of endocrine treatment of cancer based on the presence and quantities of specific molecular markers, called biomarkers, present in a tumour sample of the treated patients. More specifically, the present invention relates to methods of predicting the responsiveness of breast cancer patients to endocrine therapy. The present invention also relates to methods of providing a prognosis of disease-free and distant metastasis-free survival for a cancer patient.

### BACKGROUND OF THE INVENTION

Breast cancer has now become the most commonly diagnosed cancer among women, both in developed and developing countries, with an estimated 1.38 million new cases diagnosed annually; it is the most frequent cause of cancer death in women with approximately 458,000 deaths worldwide in 2008 (Ferlay et al., 2010).
1) Endocrine therapy is the most effective form of treatment for hormone-dependent breast cancer: Breast cancer is a group of highly heterogeneous diseases consisting of about 20 morphologically distinct subtypes (Rosen, 2009) or at least 5 molecular subtypes (Colombo et al., 2011). Although complicated, breast cancer can be divided into two distinct subtypes: estrogen receptor positive (ER+) and negative (ER−) breast cancer, with approximately 75% breast cancers being ER+. Endocrine therapies, sometimes called hormonal therapies, remain the most effective form of systemic therapy for ER+ patients (Pritchard, 2005). Current endocrine therapies include the use of selective ER modulators (e.g. tamoxifen that competes with estrogen for ER binding (Dowsett et al., 2005), and fulvestrant that down-regulates ER (Osborne et al., 2004)) or aromatase inhibitors (e.g. letrozole, anastrozole and exemestane which block the production of estrogen (Geisler et al., 2002)). Endocrine therapies have reduced the death rate associated with breast cancer and have improved overall survival. For example, adjuvant therapy with tamoxifen for 5 years results in a reduction in the annual breast cancer death rate of 34%, with an absolute reduction in mortality of 9.2% at 15 years (Early Breast Cancer Trialists' Collaborative Group (EBCTCG), 2005).
2) ER, the current clinical predictive biomarker for endocrine therapy, is not always an accurate predictor due to de novo or acquired resistance: Since the actions of endocrine therapies are to interfere with the stimulating effect of estrogen on the growth and progression of tumours, ER has been clinically used as a biomarker to predict the response of patients to endocrine therapies. However, the predictive accuracy of ER is not satisfactory because: A) although patients whose tumours are ER+ respond more frequently to the treatment than those whose tumours are ER-, the expression of ER is not always associated with treatment sensitivity; B) a significant proportion of ER+ breast tumours initially responsive to these therapies develop resistance (Clarke et al., 2001) and up to 40-50% of patients relapse (Ma et al., 2009); and C) while approximately 20% of breast cancer patients treated with endocrine therapy lose ER expression (Encarnacion et al., 1993; Gutierrez et al., 2005), most tumours that become anti-estrogen resistant still express ER (Clarke et al., 2003) and demonstrate earlier metastatic recurrence (Early Breast Cancer Trialists' Collaborative Group (EBCTCG), 2005). Therefore, lack of good predictive biomarker and endocrine resistance are two major problems in the clinical management of breast cancer.
3) Currently, there is still no clear method to distinguish tumours that will or will not respond to endocrine therapies (Al et al., 2011; Baumgarten and Frasor, 2012): During the last decade, advances in molecular biology, technology and bioinformatics have led to the advent of a new era loosely described as “omics” such as genomics (for DNA), transcriptomics (RNA), proteomics (protein), and metabolomics (metabolites). These platforms have generated a huge amount of multi-dimensional data which have been used to generate multigene profiles or signatures to predict endocrine therapy response (Musgrove and Sutherland, 2009). However, almost all of them face common issues such as insufficiently high levels of evidence, overfitting computational models and false discovery rates (Hayes and Khoury, 2012), and often these signatures do not yield significant improvement in predictive accuracy over the well-established pathological parameters such as histological grade (Clarke et al., 2003; Fan et al., 2006; Yu et al., 2007; Thomassen et al., 2007; Haibe-Kains et al., 2008; Wirapati et al., 2008; Sgroi, 2009; Prat et al., 2012). Moreover, such an approach of large scale gene expression profiles is less likely to be implemented in the clinic in the near future in comparison to conventional IHC staining. Therefore, there is an urgent need to develop a robust clinical biomarker to predict response to endocrine therapies in order to improve breast cancer management.

4) ER-regulated genes offer a promise for a better prediction of endocrine response: The precise mechanisms that contribute to progression to acquired endocrine therapy resistance are not yet fully understood. Several molecular mechanisms have been proposed to be responsible for endocrine resistance (Clarke et al., 2001; Clarke et al., 2003; Riggins et al., 2005; Giuliano et al., 2011). Because ER signalling is the target of endocrine therapy and the expression of ER is still observed in most tumours that have become resistant to endocrine therapy (Clarke et al., 2003), ER-regulated functions appear to play an important role to determine the response to the therapy. OncotypeDX, the only multigene test approved by the FDA, is a RT-PCR-based multigene assay which measures ER mRNA levels as well as the expression of several downstream ER-regulated genes (PR, BCL-2 and SCUBE2), then computes a recurrence score using an algorithm to predict patients' response to tamoxifen. Although OncotypeDX has not been widely used in clinics because it may provide no new biological insights into tamoxifen response than the measurement of ER and PR levels by the easy conventional IHC (Kok and Linn, 2010), it has been shown to accurately identify a group of patients with excellent prognosis when treated with adjuvant tamoxifen (Paik et al., 2004; Paik et al., 2006). Therefore, ER-driven genes may be promising in the development of molecular biomarkers predicting response to endocrine treatment.

### OBJECT OF THE INVENTION

It is an object of the present invention to address the foregoing disadvantages or at least to provide us with a useful choice.

### SUMMARY OF INVENTION

The present invention is based on the discovery of a novel estrogen regulated oncogene, named SHON (secreted hominoid-specific oncogene), in mammary carcinoma (Jung, et al., SHON is a novel estrogen regulated oncogene in mammary carcinoma that predicts patient response to endocrine therapy. Cancer Research 2013, in press). SHON has three transcript variants which code three SHON protein isoforms. SHON is highly expressed in all cancer cell lines tested so far, including breast, lung, liver, stomach, colon and prostate cancer. SHON, which among other things, acts to promote cell proliferation, anchorage-independent growth, colony formation, survival, migration, and invasion of cancer cells. More importantly, SHON expression is observed in 62% of breast tumours and is highly positively correlated to the expression of ER, progesterone (PR) and androgen (AR) receptors, and BCL-2, while negatively to EGFR and HER2 expression, and triple negative phenotype. Moreover, SHON expression in ER-positive (ER+)/high risk (Nottingham Prognostic Index (NPI) scores of ≥3.4) tumours is able to predict the patient response to endocrine therapy; patients whose tumours were SHON negative (SHON−) had a 2-fold increase in risk of death, recurrence and distant metastasis at 10 years compared with patients whose tumours were SHON positive (SHON+). As such, SHON provides an ideal clinical prognostic biomarker for response to endocrine therapy in high risk ER+ breast cancer patients.

### DISCLOSURE OF THE INVENTION

In one aspect, the invention provides a method of predicting the responsiveness to endocrine therapy of a tumour, which comprises obtaining a sample from the patient and determining the expression of a polypeptide containing at least a substantial part of the amino acid sequence of SEQ ID NO: 2, 5 and 6 of the accompanying drawings, or a polypeptide homologous therewith, wherein an expression of SHON polypeptide in the sample indicates that the patient is endocrine therapy-responsive and lack of the expression of SHON polypeptide in the sample indicates that the patient is endocrine therapy-resistant.

In another aspect, this invention provides a method of providing a prognosis of disease-free survival of a tumour, which comprises obtaining a sample from the patient and determining the expression of a polypeptide containing at least a substantial part of the amino acid sequence of SEQ ID NO: 2, 5 and 6 of the accompanying drawings, or a polypeptide homologous therewith, wherein an expression of SHON polypeptide in the sample indicates the good prognosis of a prolonged disease-free survival and lack of the expression of SHON polypeptide in the sample indicates the prognosis of a low disease-free survival.

In another aspect, this invention provides a method of predicting the propensity for distant metastatic spread of a tumour, which comprises obtaining a sample from the patient and determining the expression of a polypeptide containing at least a substantial part of the amino acid sequence of SEQ ID NO: 2, 5 and 6 of the accompanying drawings, or a polypeptide homologous therewith, wherein an expression of SHON polypeptide in the sample indicates that the patient the prognosis of a prolonged distant metastasis-free survival and lack of the expression of SHON polypeptide in the sample indicates the prognosis of a low distant metastasis-free survival.

In one embodiment of the invention, the sample is a fluid, a tissue or a cell. In another embodiment of the invention, the SHON polypeptide comprises an amino acid sequence substantially similar to the amino acid sequence of SEQ ID NO: 2, 5 and 6 of the accompanying drawings, or a polypeptide homologous therewith. In another specific embodiment, the SHON polypeptide is an alternatively spliced form of SHON. In a specific embodiment of the invention, the cancer comprises an ER-positive cancer or a PR-positive cancer. In a specific embodiment of the invention, the cancer is other cancers including but not limited to lung, stomach, prostate, endometrial, or ovarian cancer. A specific embodiment of the invention further comprises the step of measuring the expression of ER polypeptide, wherein an expression of ER polypeptide together with the expression of SHON polypeptide indicates that the patient is endocrine therapy-responsive, and the good prognosis of a prolonged disease-free survival and distant metastasis-free survival. In a further specific embodiment, the cancer comprises an ER-positive cancer or a PR-positive cancer.

The present invention provides a method of providing a treatment decision for a cancer patient receiving an endocrine therapy comprising obtaining a sample from the patient; and comprising the steps of obtaining a sample from the patient; and determining the expression of SHON polypeptide level in the sample, wherein the expression of SHON polypeptide indicates that that cancer is endocrine therapy responsive.

Expression of the polypeptides may be determined directly or indirectly. For example, the sample may be contacted with an antibody (monoclonal or polyclonal) specific to the selected polypeptide. Alternatively, the sample may be contacted with a nucleic acid hybridization probe capable of hybridising with the mRNA corresponding to the selected polypeptide. Still further, the sample may be subjected to a Northern blotting technique to examine for mRNA, indicating expression of the polypeptide. For those techniques in which the mRNA is detected, the sample may be subjected to a nucleic acid amplification process whereby the mRNA molecule or a selected part thereof is amplified using appropriate nucleotide primers.

The invention also features these methods of determining the effectiveness of endocrine therapy treatments of cancer by monitoring SHON expression in a subject, in particular, breast cancer, colon cancer, prostate cancer, endometrial cancer, lung cancer, stomach cancer, liver cancer, ovarian cancer or another cancer, comprising the method comprising contacting at least one antibody or antibody fragment as described herein in contact with a sample from the subject; and, determining the level of polypeptide of SHON, e.g., any one of the amino acid sequence of SEQ ID NO: 2, 5 and 6 of the accompanying drawings, or a polypeptide homologous therewith.

As another feature, the invention encompasses a method of diagnosing or monitoring cancer in a subject, in particular, breast cancer, colon cancer, prostate cancer, endometrial cancer, lung cancer, stomach cancer, liver cancer, ovarian cancer or another cancer, comprising the method comprising contacting at least one antibody or antibody fragment as described herein in contact with a sample from the subject; and, determining the level of polypeptide of SHON, e.g., any one of the amino acid sequence of SEQ ID NO: 2, 5 and 6 of the accompanying drawings, or a polypeptide homologous therewith.

The invention also features a composition of the invention as part of a kit for diagnosis or treatment, especially for cancer, and particularly breast cancer, colon cancer, prostate cancer, endometrial cancer, lung cancer, stomach cancer, liver cancer, ovarian cancer, or another cancer, in accordance with the disclosed methods. The kits can comprise: at least one component for SHON (e.g., an antibody or antibody fragment) as set out herein; and optionally, instructions for use, for example, in diagnosing or treating cancer.

Other embodiments, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this

### DETAILED DESCRIPTION

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

This invention particularly features three SHON transcript variants a, b and c, having GenBank Accession Nos. JX965369, JX965370 and JX965371, respectively, deposited on the 13 of Oct. 2012.

Other aspects and embodiments of the invention are described herein below.

### BRIEF DESCRIPTION OF THE DRAWINGS

These and other aspects of the present invention, which should be considered in all its aspects, will become apparent from the following description, which is given by way of example only, with reference to the accompanying figures:

FIG. 1. The nucleotide sequence of SHON transcript a. The SHONa sequence was compiled according to the sequences obtained in the 5′RACE as well as those of an EST clone (GenBank accession number AY358103) and a genomic clone (GenBank accession number NT_007933). Two transcription initiation sites identified in the 5′RACE at nucleotide position 1 (T) and 21 (C), respectively, are in bold. The primers used in the 5′RACE and RT-PCR are also indicated above the sequence. The translation initiation codon ATG and the termination codon TGA are underlined and in bold. The standard AATAAA polyadenylation signal is also underlined and in italics. * indicates the possible position of poly(A) tails. (SHON transcript variant a has been deposited into GenBank on the 13 of Oct. 2012 with Accession No. JX965369).

FIG. 2. Predicted amino acid sequence of SHONα protein. The protein sequence is presented in single letter code according to the nucleotide sequence of SHON transcript a.

Predicted signal peptide (residues 1-21) is in lower case. The cysteines are in bold italics. A postulated internal disulfide bond between the two cysteines in matured protein is also labelled. Amino acid numbers are in the left margin. The sequence was analysed for conserved motifs using Motif Scan (MyHits, Swiss Institute of Bioinforrnatics; Lausanne, Switzerland), SOSUI (Mitalm Group; Tokyo, Japan) and PredictProtein (BiosofLLC; New York, N.Y., USA).

FIGS. 3A-B. The nucleotide sequence of SHON transcripts b and c. The sequence was compiled according to the sequences obtained in the 5′RACE as well as those of an EST clone (IMAGE:1286243) from human tonsillar cells (GenBank accession numbers CR745472 and AA740612) and a genomic clone (GenBank accession number NT_007933). Two additional SHON transcript variants were identified: a longer one (SHONb) (A) and a shorter one (SHONc) (B). Compared with transcript b, transcript c has a shorter exon 2 as a result of alternative splicing using a downstream acceptor. The sequence of 118 bp in length unique to transcript variant b is in lower case. The transcription initiation site T at nucleotide position 21 identified in the 5′RACE is in bold. The primers used in the 5′RACE and RT-PCR are also indicated above the sequence. Three potential in frame translation initiation codons ATG and the termination codon TAG are underlined and in bold. The standard AATAAA polyadenylation signal is also underlined and in italics. ▾, indicates the position of the intron of 5,000 bp; * indicates the possible position of poly(A) tails. (SHON transcript variants b and c have been deposited into GenBank on the 13 of Oct. 2012 with Accession No. JX965370 and JX965371, respectively).

FIGS. 4A-B. Predicted amino acid sequence of SHON protein isoforms β and γ. The protein sequences are presented in single letter code according to the nucleotide sequence of SHON transcripts b and c, respectively. The cysteines are in bold italics. A postulated internal disulfide bond between the two cysteines in matured protein is also labelled. Amino acid numbers are in the left margin. The sequence was analysed for conserved motifs using Motif Scan (MyHits, Swiss Institute of Bioinformatics; Lausanne, Switzerland), SOSUI (Mitaku Group; Tokyo, Japan) and PredictProtein (Biosof LLC; New York, N.Y., USA).

FIG. 5. Alignment of SHON protein isoforms. Alignment of SHON protein isoforms α, (SEQ ID NO: 2), β (SEQ ID NO: 5), and γ (SEQ ID NO: 6) coded by transcripts a, band c, respectively, was carried out using CLUSTAL v2.0.10 (Conway Institute-University College Dublin; Dublin, Ireland) with manual adjustments. The aligned sequences were shaded using BOXSHADE v3.31C (available from sourceforge.net, SlashdotMedia; La Jolla, Calif., USA).

FIGS. 6A-B. Genomic description of SHON locus. (A) Schematic structure of the SHON locus showing the relative positions of SHON transcripts a, band c and the two promoters. Transcript a contains a single exon (light shaded box) and uses promoter 1, while both transcripts band c have two exons, which results from splicing by the use of a downstream acceptor, and are under the control of promoter 2. These exons (E1, E2 or E2′) are indicated by solid or dark shaded boxes, and introns by empty boxes. The promoter sequences are annotated with TATA, CAAT and GC boxes. The transcription initiation sites and the position of the conserved polyadenylation signal AATAAA are also indicated. The sizes of exons and introns are marked. The drawings are not to scale. (Figure discloses “cacagccaat gg” as SEQ ID NO: 37) (B and C) Sequence of SHON gene extracted from genomic clone (GenBank accession number NT 007933). Introns are in lower case with splicing donor (GT) and acceptor (AG) sites highlighted in bold and in italics, and exons are in upper case with the sequence of 118 bp in length unique to transcript variant SHONb is underlined. Numbers at the left indicate the position of the sequence relative to the transcription start site (+1) of respective SHON transcripts a, band c. Some of the potential transcription factor binding sites defined by using the TFSEARCH online database (Parallel Application TRC Laboratory, Real World Computing Project, Japan) are annotated. Dozens of potential Sp1 sites also were detected in promoter 1 but not drawn. The arrows indicate the strand polarity. The transcription initiation sites are in bold and in italics. Translation start codons ATG and the termination codon TGA, and the conserved polyadenylation signal AATAAA are underlined and in bold. The termination codon TAG for SHONc is boxed. The promoter sequence is annotated with TATA box, CAAT box and GC box. Primers used to confirm the transcription variants are also annotated.

FIG. 7. SHON mRNA expression in MCF-7 cells. SHON mRNA transcript variants were amplified by PCR from MCF-7 cells after reverse transcription in the presence (+) or absence (−) of reverse transcriptase (RT) as indicated. Two pairs of primers SHONc5/SHONc3 and SHONF1/SHONc3 (Table 1) were used to amplify transcripts for SHON a/b (of 280 bp for both) and b/c (456 bp for b and 338 bp for c), respectively. M, 1 Kb Plus DNA Ladder.

FIGS. 8A-C: Characterization of a polyclonal anti-SHONα antibody raised in rabbits. (A) Western blotting. E. coli M15 cells harbouring the pQE30-SHONα plasmid (coding a HIS-tagged SHONα fusion protgein HIS-SHONα) prior to (−) and post (+) the addition of IPTG, HIS-SHONα bound Ni-NTA beads and HIS-SHONα protein elutes were separated by SDS-PAGE and transferred to PVDF membranes, then probed with the mouse anti-HIS monoclonal antibody. HIS-SHONα was detected as a band of approximately 6 kDa. (B) Western blotting. Purified recombinant GST-SHONα fusion protein (5 ng each lane) was separated by SDS-PAGE and immunoprobed with rabbit sera collected prior to immunization (Pre), 3 weeks after the first immunization (Post 1), and 3 weeks after the second boost immunization (Post 2). The anti-sera clearly recognized GST-SHONα fusion and fragments of SHONα. (C) Western blotting. Purified HIS-SHONα was immunoprobed with the anti-SHONα serum raised with the GST-SHONα in (B). The anti-sera were able to detect the HIS-SHONα as a band of about 6 kDa. Molecular weights (M) of detected protein bands in kDa are shown on the left.

FIGS. 9A-E. Specificity of the rabbit SHONα polyclonal antibody. (A) MCF-7 cells were transiently transfected with the SHONα expression plasmid pIRESneo3-SHONα at indicated amounts for 24 h. Cells were then lysed for Western blot analysis. The blot was immuno-blotted with the rabbit polyclonal SHONα antibody. The forced expression of SHONα from the plasmid was detected as a specific band of 12 kDa. (B) MCF-7 cells were stably transfected with the SHONα expression plasmid pIRESneo3-SHONα (MCF7-SHON) or the empty vector plasmid pIRESneo3 (MCF7-Vec) as a control, or with the SHON siRNA plasmid pSilencer-siRNA (MCF7-siRNA) or the negative siRNA control plasmid pSilencer-CK (MCF7-CK) as indicated. Reverse transcription (RT)-PCR was performed to detect the expression of SHON mRNA, and Western blot (WB) was done for the detection of SHON protein using the rabbit polyclonal SHONα antibody. (C) Peptide blocking. Whole cell lysates of stable MCF7-Vec (Vector) and MCF7-SHON (SHON) cells at indicated amounts were immune-blotted with 1 μg/m1 of the affinity purified SHONα antibody which was pre-incubated with 1 μg/ml of BSA or recombinant HIS-SHONα peptide (HIS-SHON). (D) Immunoprecipitation. The whole cell lysates of stable MCF7-Vec (Vector) and MCF7-SHON (SHON) cells were immuno-precipitated with affinity purified SHONα antibody (anti-SHON) and normal control rabbit IgG (Control IgG). The precipitants were subjected to Western blotting analysis with the SHONα antibody. Both the endogenous and forced expression of SHON proteins were effectively pulled-down by the SHONα antibody as a full-length of 12 kDa band. The 6 kDa band may be the result of degradation during immunoprecipitation. (E) SHON protein expression in a variety of human breast cell lines was detected by Western blot using affinity purified SHONα antibody. β-actin was used as a loading control. The sizes of amplified RT-PCR products or molecular weights of detected protein bands are shown on the sides.

FIG. 10A-C. SHON mRNA and protein is expressed in normal human tissues, cancer cell lines and breast cancer tissues. (A) SHON was amplified by PCR (40 cycles) with SHON-specific primers (SHONc5/SHONc3) for both transcripts a and b in a panel of cDNAs derived from different human tissues (OriGene). The tissue of origin is indicated above each lane. The GAPDH gene was used as the cDNA input control. (B) The expression of SHON mRNA in human cell lines, as indicated, was examined by RT-PCR with the SHON-specific primers. The expression of SHON protein was determined by Western blot (WB) by the rabbit anti-SHONα polyclonal antibody. β-ACTIN was included as the RNA input control or cell lysate protein input control. (C) The expression of SHON mRNA in a Breast Cancer cDNA Array (OriGene) was examined by PCR with the SHON-specific primers. The array contained 48 samples covering 5-normal, 11-stage I, 8-IIA, 6-IIB, 8-IIIA, 2-IIIB, 4-IIIC and 4-IV. (3-ACTIN was included as the cDNA input control. The sizes of amplified PCR products are shown on the right. Relative expression of SHON in the cDNA array panel was estimated by densitometric analysis using the ImageJ software (NIH) with β-ACTIN as the normalization control. The sizes of amplified (RT-)PCR products or molecular weights of detected protein bands are shown on the right.

FIGS. 11A-D. SHONα is a secreted protein and SHONβ is a proprotein. (A) Western blot analyses. HEK293 cells were transfected with a HIS-tagged SHON α expression plasmid pIRESneo3-SHONα-HIS (SHONα-HIS) or the empty control vector (Vector). Soluble whole cellular extracts or concentrated media were separated on an SDS-PAGE and immunoblotted using a mouse anti-HIS tag monoclonal antibody or the anti-SHONα polyclonal. β-ACTIN was used as loading control for cell lysates. (B) MCF-7 cells were transiently transfected with SHONα expression plasmid pIRESneo3-SHONα, SHONβ plasmid pIRESneo3-SHONβ or the empty control pIRESneo3 vector plasmid at the indicated amounts. The expression of SHON protein was analyzed by Western blot. M, Molecular mass markers indicated in kDa by bars on the left. (C) Predicted amino acid sequence of SHONP protein (SEQ ID NO: 5). Conserved motif (K/R)Xn(K/R) recognised for proprotein convertases are in bold and underlined. (D) HEK293 cells were transfected with the empty vector pIRESneo3 (Vector), SHONα expression plasmid pIRESneo3-SHONα (SHONα), SHONβ plasmid pIRESneo3-SHONβ (SHONβ), and SHONβ mutant plasmid pIRESneo3-SHONβni (SHONβmut) in which the third conserved motif K⁶²R⁶³ was mutated into N⁶²I⁶³, as well as pIRESneo3-SHONα-Myc (SHONα-Myc), pIRESneo3-SHONβ-Myc (SHONβ-Myc) and pIRESneo3-SHONβni (SHONβmut-Myc) which express a C-terminal c-Myc tagged SHONα, SHONβ and SHONβ (N⁶²I⁶³), respectively. The expression of SHON was examined by Western blot with a rabbit anti-SHON polyclonal antibody or the mouse monoclonal antibody 9E10 to the c-Myc tag. β-ACTIN was used as protein lysate loading control. Molecular weights of detected protein bands are shown on the right.

FIGS. 12A-C. SHON is an estrogen inducible gene. (A) RT-PCR. MCF-7 cells were cultured for 24 h in RPMI media containing 10% foetal bovine serum (FBS). Prior to 17β-estradiol (E2) treatment, cells were further cultured for 72 h in phenol red-free medium containing 10% charcoal stripped-FBS. Cells were then treated with 10 nM of E2. Total RNAs were isolated from the cells at indicated time points and the expression of SHON mRNA was determined by One-Step RT-PCR kits (Qiagen) with the SHON-specific primer pairs of SHONc5 and SHONc3 (Table 1). β-ACTIN was included as the RNA input control using forward primer: 5′-ATCATATCGCCGCGCTCG-3′ (SEQ ID NO: 9) and reverse primer: 5′-CGCTCGGTGAGGATCTTCA-3′ (SEQ ID NO: 10). The bp sizes of amplified products are shown on the right. (B) MCF7-Vec and MCF7-SHON cells were treated with DMSO vehicle (−) or 10 nM of E2 (+) for 72 h in phenol red-free medium containing 10% charcoal stripped-FBS. The expression of SHON in whole cell lysates was determined by Western blot with the rabbit polyclonal SHONα antibody. Molecular weights of detected protein bands are shown on the right. (C) Total cell number assays. MCF7-Vec and MCF7-SHON stable cells were seeded in phenol-red free RPMI media with 10% charcoal stripped FBS. The cells were treated with DMSO (Veh), 10 nM 17β-Estradiol (E2) or 100 nM ICI 182,780 (ICI). The cell number was determined at the days indicated. All numerical data are presented as mean±SD (standard deviation). The cell number was determined at the indicated days. **, P<0.01; ***, P<0.001.

FIGS. 13A-B. Immunocytochemistry with affinity purified SHONα antibody. (A) HEK293 cells were transiently transfected with the expression plasmid pIRESneo3-SHONα-EGFP, which encodes SHONα with a C-terminal EGFP tag, and the pEGFP-C1 empty vector, which encodes the EGFP protein. 24 h post-transfection, cells were fixed and permeabilized with Triton X-100 for immunocytochemical staining with the affinity purified rabbit polyclonal SHONα antibody as the primary antibody. The staining of SHONα was then visualised with a Cy5 cyanine dye conjugated secondary antibody (Red) and counterstained with Hoechst 33258 (Blue). The expression of EGFP or SHONα-EGFP was examined by fluorescence microscopy (Green). Merged images are shown in the far right column. The co-localisation of green and red fluorescence staining in the pIRESneo3-SHONα-EGFP transfected cells demonstrated that the rabbit anti-SHONα antibody specifically recognised SHON. Bar, 50 μm. (B) HEK293 cells were transiently transfected with the SHONα expression plasmid pIRESneo3-SHONα (SHON) and the empty control pIRESneo3 vector (Vector). 48 h after transfection, cells were harvested. Cell pellets were fixed with 4% paraformaldehyde and embedded in paraffin. Paraffin sections were boiled for 20 min in 10 mM of citrate buffer (pH 6.0) using a high pressure cooker for antigen retrieval, and then stained with the rabbit anti-SHONα antibody. SHON protein was visualized by using a Cy5 cyanine dye conjugated secondary antibody (Red). The nuclei were counterstained with Hoechst 33258 (Blue). Merged images are shown in the far right column. The rabbit anti-SHONα antibody was able to recognise SHON protein in the formalin-fixed paraffin-embedded cell blocks. Bar, 50 μm.

FIGS. 14A-B. Microphotographs of SHON expression in normal and breast cancer tissues. (A) IHC staining optimization controls. (B) SHON expression in representative normal and breast cancer TMA cores. ER, estrogen receptor.

FIGS. 15A-C. Kaplan-Meier survival curves. Kaplan-Meier plots of the rates of breast cancer specific survival (BCSS) (A), disease free survival (DFS) (B), and distant metastasis free survival (DMFS) (C) for all patients in the study according to SHON expression status. The p value from the log rank test is shown in each panel; ‘n’ is the number of samples in each group.

FIGS. 16A-C. SHON expression predicts patient response to endocrine therapy in ER+ breast cancer. Kaplan Meier survival curves demonstrating the rates of breast cancer specific survival (BCSS) (A), disease free survival (DFS) (B), and distant metastasis free survival (DMFS) (C) of high risk breast cancer (Nottingham Prognostic Index ≥3.4)/ER+ patients whose tumours were SHON positive (+) versus negative (−). p values represent log-rank testing of the difference in survival.

FIGS. 17A-B Kaplan-Meier survival curves. Kaplan-Meier plots of the rates of disease free survival of ER− patients who were untreated (A) or treated (B) with anthracycline in the study according to SHON expression status. The p value from the log rank test is shown in each panel; ‘n’ is the number of samples in each group.

FIGS. 18A-D. Production and specificity of mouse monoclonal antibodies against SHON. (A) Western blot. MCF-7 cells were stably transfected with the SHONα expression plasmid pIRESneo3 SHONα (SHON) or the empty vector plasmid pIRESneo3 (Vector) as a control. Soluble whole cellular extracts were separated on an SDS-PAGE and immunoblotted using the purified mouse monoclonal SHON antibodies from clones mAb#4, mAb#5, mAb#8 and mAb#8. M, Molecular weights of protein markers in kDa. (B) Western blot. SHON protein expression in a variety of human breast cell lines as indicated was detected by Western blot using the purified mouse monoclonal SHON antibodies from clone mAb#5. The blot was exposed at shorter and longer exposures. M, Molecular weights of protein markers in kDa. (C) HEK293 cells were transiently transfected with the expression plasmid pIRESneo3-SHONα-EGFP, which encodes SHONα with a C-terminal EGFP tag. 24 h post-transfection, cells were fixed and permeabilized with Triton X-100 for immunocytochemical staining with purified mouse monoclonal antibody clones 1H6 or 4G4, or mouse polyclonal SHONα antibody (mPA #4) as the primary antibody. The staining of SHONα was then visualised with a Cy3 cyanine dye conjugated secondary antibody (Red). The expression of SHONα-EGFP was examined by fluorescence microscopy (Green). Merged images are shown in the far right column. The co-localisation of green and red fluorescence staining in the pIRESneo3-SHONα-EGFP transfected cells demonstrated that the mouse anti-SHONα monoclonal antibodies specifically recognised SHON. (D) Western blot. HEK293 cells were transiently transfected with the expression plasmid pIRESneo3-SHONα-EGFP, which encodes SHONα with a C-terminal EGFP tag, and the pEGFP-C1 empty vector, which encodes the EGFP protein. 24 h post-transfection. Soluble whole cellular extracts of pIRESneo3-SHON □-EGFP transfected cells (+) or the empty vector pEGFP-C1 transfected cells (−) were separated on an SDS-PAGE and immunoblotted using the purified mouse monoclonal antibodies from subclones of the 1H6 or 4G4 clones, or the mouse polyclonal SHONα antibody (mPA #4). Lane 1, 1H6 subclone 1; Lane 2, 1H6 subclone 2; Lane 3, 1H6 subclone 3; Lanes 4 and 8, the mouse polyclonal SHONα antibody (mPA #4); Lane 5, 4G4 subclone 3; Lane 6, 4G4 subclone 4; Lane 7, 4G4 subclone 6. Molecular weights of protein markers are shown on the left.

### DETAILED DESCRIPTION OF THE INVENTION

The following is a description of the present invention, including preferred embodiments thereof, given in general terms. The invention is further elucidated from the disclosure given under the section “Examples” which provides experimental data supporting the invention and specific examples thereof.

The present invention provides clinical data and observations of ER+ breast cancer patients having tumours expressing ER-regulated genes. In particular, the expression of SHON demonstrated a surprising and significant relationship with endocrine therapy effectiveness. The identified relationship led to the development of novel methods to predict disease-free and distant metastasis-free survival of a breast cancer patient, in particular ER+ cancers. The invention establishes that the ER-regulated SHON is an important molecular marker for determining disease-free and distant metastasis-free survival, for identifying endocrine therapy-response patients and for treating those patients. Specifically, SHON gene expression levels serve as a predictor of endocrine therapy response and as a prognostic marker. In contrast, SHON polypeptide expression predicts favourable disease-free and distant metastasis-free survival in patients not receiving endocrine therapy, and serves as a prognostic marker in patients receiving endocrine therapy.

Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “disease-free survival” as used herein is defined as the time between the first diagnosis and/or first surgery to treat a cancer patient and a first reoccurrence. For example, a disease-free survival is “low” if the cancer patient has a first reoccurrence within five years after tumour resection, and more specifically, if the cancer patient has less than about 55% disease-free survival over 5 years. For example, a high disease-free survival refers to at least about 55% disease-free survival over 5 years.

The term “breast cancer-specific survival” is defined as the time from and/or first surgery to treat a cancer patient to the time of a breast cancer-related death. For instance, long-term breast cancer-specific survival is for at least 5 years, more preferably for at least 8 years, most preferably for at least 10 years following surgery or other treatment.

The term “distant metastasis free survival” is defined as the time from diagnosis and/or first surgery to treat a cancer patient to the time of first distant metastasis.

The term “endocrine therapy” as used herein is defined as a treatment of or pertaining to any of the ducts or endocrine glands characterized by secreting internally and into the bloodstream from the cells of the gland. The treatment may remove the gland, block hormone synthesis, or prevent the hormone from binding to its receptor.

The term “endocrine therapy-responsive patient” as used herein is defined as a patient receiving an endocrine therapy and demonstrating a desired physiological effect, such as a therapeutic benefit, from the administration of an endocrine therapy.

The term “SHON” as used herein refers to, but not limited to, SHON transcript variant a, SHON transcript variant b, and SHON transcript variant c; and SHON protein isoform α, SHON protein isoform β, and SHON protein isoform γ. For use with the invention, human sequences are preferred, but other homologs and orthologs can also be used. It will be understood that each reference to these factors (e.g., SHON or like terms), herein, will include the full length sequences as well as any fragments, or modifications (including variants) thereof.

The term “SHON positive (SHON+)” as used herein refers to cancers that do have SHON while those cancers that do not possess SHON are “SHON-negative (SHON−).”

The term “estrogen-receptor positive (ER+)” as used herein refers to cancers that do have estrogen receptors while those cancers that do not possess estrogen receptors are “estrogen receptor-negative (ER−).”

The term “polypeptide” as used herein is used interchangeably with the term “protein” and is defined as a molecule which comprises more than one amino acid subunits. The polypeptide may be an entire protein or it may be a fragment of a protein, such as a peptide or an oligopeptide. The polypeptide may also comprise alterations to the amino acid subunits, such as methylation or acetylation. The polypeptide may be naturally occurring, recombinant, synthetic, or semi-synthetic molecules. Where these terms are recited herein to refer to an amino acid sequence of a naturally occurring protein molecule, the terms are not meant to limit the amino acid sequence to the complete, original sequence for the full length molecule. It will be understood that each reference to “polypeptide” or like term, herein, will include the full length sequence, as well as any modifications thereof.

The term “prognosis” as used herein is defined as a prediction of a probable course and/or outcome of a disease. For example, in the present invention SHON is a prognostic marker for response to endocrine therapy in a cancer patient.

The term “prediction” is used herein to refer to the likelihood that a patient will respond either favourably or unfavourably to a drug or set of drugs, and also the extent of those responses, or that a patient will survive, following surgical removal or the primary tumour and/or chemotherapy for a certain period of time without cancer recurrence. The predictive methods of the present invention are valuable tools in predicting if a patient is likely to respond favourably to a treatment regimen, such as surgical intervention, chemotherapy with a given drug or drug combination, and/or radiation therapy, or whether long-term survival of the patient, following surgery and/or termination of chemotherapy or other treatment modalities is likely.

“SEQ ID NO:” as referred to herein, can indicate each sequence identifier individually, or any combination thereof, or all such sequence identifiers.

The term “substantially similar to SEQ ID NO: 2, 5 and 6”, for example, as used herein is defined as a polypeptide having an amino acid sequence that is at least about 70% identical to or similar to SEQ ID NO: 2, 5 and 6, and the substantially similar polypeptide also exhibits the biological activity of the polypeptide of SEQ ID NO: 2, 5 and 6.

The term “treatment” and like terms refer to methods and compositions to prevent, cure, or ameliorate a medical disorder (e.g., medical disease, condition, or syndrome), or reduce at least a symptom of such disorder. In particular, this includes methods and compositions to prevent or delay onset of a medical disorder; to cure, correct, reduce, slow, or ameliorate the physical or developmental effects of a disorder; and/or to prevent, end, reduce, or ameliorate the pain or suffering caused the disorder. The term “treatment” is to be considered in its broadest context. The term does not necessarily imply that the subject is treated until total recovery. Accordingly, “treatment” as used herein broadly includes inhibiting, reducing or preventing cell proliferation, cell survival, cell motility, and/or oncogenicity; ameliorating the symptoms or severity of cell proliferation, cell survival, cell motility, and/or oncogenicity; or preventing or otherwise reducing the risk of developing cell proliferation, cell survival, cell motility, and/or oncogenicity, for example cancer, and in particular, breast cancer, colon cancer, prostate cancer, endometrial cancer, lung cancer, stomach cancer, liver cancer, ovarian cancer, or another cancer.

The term “sample” as used herein indicates a patient sample. Tissue, cell, or fluid samples can be removed from almost any part of the body. The most appropriate method for obtaining a sample depends on the type of cancer that is suspected or diagnosed. Biopsy methods include needle, endoscopic, and excisional. The treatment of the tumour sample or body fluid after removal from the body depends on the type of detection method that will be employed for determining SHON or ER expression.

The term “antibody” should be understood in the broadest possible sense and is intended to include intact monoclonal antibodies and polyclonal antibodies. It is also intended to cover modified antibodies so long as they exhibit the desired biological activity. Antibodies encompass immunoglobulin molecules and immunologically active portions of immunoglobulin (Ig) molecules, i.e., molecules that contain an antigen binding site that specifically binds (immunoreacts with) an antigen. It will be understood that each reference to “antibodies” or any like term, herein includes intact antibodies, as well as any modifications thereof

The term “amino acid sequence”, as used herein, refers to a sequence of an oligopeptide, peptide, polypeptide, protein, or antibody, and any fragment thereof, and to any naturally occurring, recombinant, synthetic, or semi-synthetic molecules. The sequences of the invention comprise at least 5, 6, 7, 8, 9, 10, 11, or 12 amino acids, preferably at least 5 to 10, 5 to 15, 10 to 15, or 12 to 15 amino acids. Preferably, the sequences retain the biological activity (e.g., effect on cell proliferation, cell survival, cell motility, and/or oncogenicity) or the immunogenicity/immunological activity of the original amino acid sequence. It will be understood that “amino acid sequence” and like terms are not limited to the complete, original sequence associated with the full-length molecule, but include also any modifications thereof

The term “expression” includes production of polynucleotides and polypeptides, in particular, the production of RNA (e.g., mRNA) from a gene or portion of a gene, and includes the production of a polypeptide encoded by an RNA or gene or portion of a gene, and the appearance of a detectable material associated with expression. For example, the formation of a complex, for example, from a polypeptide-polypeptide interaction, polypeptide-nucleotide interaction, or the like, is included within the scope of the term “expression”. Another example is the binding of a binding ligand, such as a hybridization probe or antibody, to a gene or other polynucleotide or oligonucleotide, a polypeptide or a protein fragment, and the visualization of the binding ligand. Thus, increased intensity of a spot on a microarray, on a hybridization blot such as a Northern blot, or on an immunoblot such as a Western blot, or on a bead array, or by PCR analysis, is included within the term “expression” of the underlying biological molecule.

The term “homology”, as used herein, refers to a degree of complementarity. There may be partial homology (i.e., a certain % identity) or complete homology (i.e., 100% identity). A partially complementary sequence that at least partially inhibits an identical sequence from hybridizing to a target nucleic acid is referred to using the functional term “substantially homologous.” The inhibition of hybridization of the completely complementary sequence to the target sequence may be examined using a hybridization assay (e.g., Southern or northern blot, solution hybridization, and the like) under conditions of low stringency. A substantially homologous sequence or hybridization probe will compete for and inhibit the binding of a completely homologous sequence to the target sequence under conditions of low stringency. This is not to say that conditions of low stringency are such that non-specific binding is permitted; low stringency conditions require that the binding of two sequences to one another be a specific (i.e., selective) interaction.

The term “hybridization”, as used herein, refers to any process by which a strand of nucleic acid binds with a complementary strand through base pairing.

The terms “modified” or “modification” refer to altered sequences and to sequence fragments, variants, and derivatives, as described herein. The term includes polypeptides, polynucleotides, antibodies, and like agents described herein.

The term “nucleic acid sequence” or “nucleotide sequence” as used herein, refers to a sequence of a polynucleotide, oligonucleotide, or fragments thereof, and to DNA or RNA of natural, recombinant, synthetic or semi-synthetic, origin which may be single or double stranded, and can represent sense or antisense strand, or coding or non-coding regions. The sequences of the invention, preferably, comprise at least 15, 21, 27, 33, 36, 39, 45, 51, 57, or 66 nucleotides, preferably at least 15 to 36, 15 to 66, 36 to 66, or 45 to 66 nucleotides, or at least 100 nucleotides, or at least 1000 nucleotides. It will be understood that each reference to a “nucleic acid sequence” or “nucleotide sequence,” herein, will include the original, full length sequence, as well as any complements or modifications thereof. It will be further understood that any reference to a “polynucleotide” (or “oligonucleotide,” or “probe,” or “primer,” etc.) having a particular SEQ ID NO. will encompass both the DNA and the counterpart RNA sequences.

The term “oligonucleotide” refers to a polynucleotide, typically a probe or primer, including, without limitation, single stranded DNAs, single or double stranded RNAs, RNA:DNA hybrids, and double stranded DNAs. Oligonucleotides, such as single stranded DNA probe oligonucleotides, are often synthesized by chemical methods, for example using automated oligonucleotide synthesizers that are commercially available, or by a variety of other methods, including in vitro expression systems, recombinant techniques, and expression in cells and organisms.

A “variant” of polypeptide, as used herein, refers to an amino acid sequence that is altered by one or more amino acids. Similarly, a variant antibody is altered by one or more amino acids. A variant polynucleotide is altered by one or more nucleotides. A variant may result in “conservative” changes, wherein a substituted amino acid has similar structural or chemical properties, e.g., replacement of leucine with isoleucine. More rarely, a variant may result in “nonconservative” changes, e.g., replacement of a glycine with a tryptophan. Analogous minor variations may also include amino acid deletions or insertions, or both. Guidance in determining which amino acid residues may be substituted, inserted, or deleted without abolishing biological or immunogenic activity may be found using computer programs well known in the art, for example, LASERGENE software (DNASTAR).

The invention also encompasses variants which retain at least one biological activity (e.g., effect on cell proliferation, cell survival, cell motility, and/or oncogenicity) or immunogenic/immunological function. A preferred variant is one having at least 80%, and more preferably at least 90%, sequence identity to a disclosed sequence. A most preferred variant is one having at least 95%, at least 97%, at least 98%, or at least 99% sequence identity to a sequence disclosed herein. The percentage identity is determined by aligning the two sequences to be compared as described below, determining the number of identical residues in the aligned portion, dividing that number by the total number of residues in the inventive (queried) sequence, and multiplying the result by 100. A useful alignment program is AlignX (Vector NTI).

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, Molecular Cloning: A Laboratory Manual, 2nd edition, Sambrook et al., 1989; also, Molecular Cloning: A Laboratory Manual, 3^{rd} Edition, Sambrook et al., 2000; Oligonucleotide Synthesis, MY Gait, ed., 1984; Animal Cell Culture, R. I. Freshney, ed., 1987; Methods in Enzymology, Academic Press, Inc.; Handbook of Experimental Immunology, 4th edition, D. M. Weir & C C. Blackwell, eds., Blackwell Science Inc., 1987; Gene Transfer Vectors for Mammalian Cells, JAM. Miller & MAP. Calos, eds., 1987; Current Protocols in Molecular Biology, FEM. Ausubel et al., eds., 1987; and PCR: The Polymerase Chain Reaction, Mullis et al., eds., 1994.

Any of the methods described herein may be implemented using therapeutic compositions of the invention and vice versa. It is contemplated that any embodiment discussed with respect to an aspect of the invention may be implemented or employed in the context of other aspects of the invention.

Compositions for Detection of SHON

The agent for use in detecting SHON (e.g., an antibody or antibody fragment) may be used on its own, or in the form of compositions in combination with one or more pharmaceutically acceptable diluents, carriers, and/or excipients.

SHON Polynucleotides and Polypeptides

The invention employs polypeptides and peptides for producing SHON antibodies, including those directed to at least one of SEQ ID NO: 2, 5 and 6 of the accompanying drawings, and fragments, and modifications thereof. SHON antibodies can be used in conjunction with these polypeptides in the diagnosis of cancer, especially breast cancer, colon cancer, prostate cancer, endometrial cancer, lung cancer, stomach cancer, liver cancer, ovarian cancer, or another cancer. The polypeptides may be used for large-scale synthesis and isolation protocols, for example, for commercial production.

The polypeptides of the present invention comprise at least one sequence selected from the group consisting of: (a) polypeptides comprising at least one amino acid sequence selected from the group consisting of SEQ ID NO: SEQ ID NO: 2, 5 and 6 or modifications thereof; (b) polypeptides comprising a functional domain of at least one amino acid sequence selected from the group consisting of SEQ ID NO: 2, 5 and 6, and modifications thereof; and (c) polypeptides comprising at least a specified number of contiguous residues of at least one amino acid sequence selected from the group consisting of SEQ ID NO: 2, 5 and 6, or modifications thereof. In one particular embodiment, the invention encompasses an isolated polypeptide comprising the amino acid sequence of at least one of SEQ ID NO: 2, 5 and 6. All of these sequences are collectively referred to herein as polypeptides of the invention.

The invention also encompasses polynucleotides for producing SHON antibodies, including those directed to the coding sequences of SEQ ID NO: 1, 3 and 4, and modified sequences thereof. Accordingly, the invention encompasses the use of the polynucleotides for preparing expression vectors and host cells, and for preparing antisense polynucleotides and iRNAs. The polynucleotides of the present invention may also be used as compositions, for example, pharmaceutical compositions.

The polynucleotides of the present invention comprise at least one sequence selected from the group consisting of: (a) sequences comprising a coding sequence for at least one amino acid sequence selected from the group consisting of SEQ ID NO: 1, 3 and 4, or modifications thereof; (b) complements, reverse sequences, and reverse complements of a coding sequence for at least one amino acid sequence selected from the group consisting of SEQ ID NO: 1, 3 and 4, or modifications thereof; (c) open reading frames contained in the coding sequence for at least one amino acid sequence selected from the group consisting of SEQ ID NO: 1, 3 and 4, or their modifications (d) functional domains of a coding sequence for at least one amino acid sequence selected from the group consisting of SEQ ID NO: 1, 3 and 4, or modifications thereof, (e) sequences comprising at least a specified number of contiguous residues of a coding sequence for at least one amino acid sequence selected from the group consisting of SEQ ID NO: 1, 3 and 4, or modifications thereof; and (0 sequences comprising at least a specified number of contiguous nucleotides of SEQ ID NO: 1, 3 and 4, or complements, or modified sequences thereof. In one particular embodiment, the invention encompasses an isolated polynucleotide comprising a coding sequence for at least one amino acid sequence selected from the group consisting of SEQ ID NO: 1, 3 and 4. In another particular embodiment, the invention encompasses an isolated polynucleotide comprising a sequence selected from the group consisting of SEQ ID NO: SEQ ID NO: 1, 3 and 4. Oligonucleotide probes and primers and their modifications are also provided. All of these polynucleotides and oligonucleotide probes and primers are collectively referred to herein, as polynucleotides of the invention.

It will be appreciated by those skilled in the art that as a result of the degeneracy of the genetic code, a multitude of nucleotide sequences encoding the polypeptides of the invention, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to naturally occurring amino acid sequences, and all such variations are to be considered as being specifically disclosed.

Nucleotide sequences for SHON, or modifications thereof, are preferably capable of hybridizing to the nucleotide sequence of the naturally occurring sequence under appropriately selected conditions of stringency. However, it may be advantageous to produce nucleotide sequences, or modifications thereof, possessing a substantially different codon usage. Codons may be selected to increase the rate at which expression of the polypeptide occurs in a particular prokaryotic or eukaryotic host in accordance with the frequency with which particular codons are utilized by the host. Other reasons for substantially altering the nucleotide sequence and its derivatives without altering the encoded amino acid sequences include the production of RNA transcripts having more desirable properties, such as a greater half-life, than transcripts produced from the naturally occurring sequence.

The invention also encompasses production of polynucleotides for SHON, or modifications thereof, entirely by synthetic chemistry. After production, the synthetic sequence may be inserted into any of the many available expression vectors and cell systems using reagents that are well known in the art. Moreover, synthetic chemistry may be used to introduce mutations into a nucleotide sequence, or any derivatives thereof. Also encompassed by the invention are polynucleotide sequences that are capable of hybridizing to the claimed nucleotide sequences, and in particular, those shown in SEQ ID NO: 1, 3 and 4, or complements, or modified sequences thereof, under various conditions of stringency as taught in Wahl, G. M. and S. L. Berger (1987; Methods Enzymol. 152:399-407) and Kimmel, A. R. (1987; Methods Enzymol. 152:507-511).

Methods for DNA sequencing which are well known and generally available in the art may be used to practice any of the embodiments of the invention. The methods may employ such enzymes as the Klenow fragment of DNA polymerase I, SEQUENASE (U.S. Biochemical Corp, Cleveland, Ohio), Taq polymerase (Perkin Elmer), thermostable T7 polymerase (Amersham Pharmacia Biotech, Piscataway, N.J.), or combinations of polymerases and proofreading exonucleases such as those found in the ELONGASE Amplification System (Life Technologies, Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) the ABI Catalyst and 373 and 377 DNA Sequencers (Perkin Elmer), or the Genome Sequencer 20™ (Roche Diagnostics).

The nucleic acid sequences may be extended utilizing a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). In particular, genomic DNA is first amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Capillary electrophoresis systems which are commercially available may be used to analyse the size or confirm the nucleotide sequence of sequencing or PCR products. In particular, capillary sequencing may employ flowable polymers for electrophoretic separation, four different fluorescent dyes (one for each nucleotide), which are laser activated, and detection of the emitted wavelengths by a charge coupled device camera. Output/light intensity may be converted to electrical signal using appropriate software (e.g., GENOTYPER and Sequence NAVIGATOR, Perkin Elmer) and the entire process from loading of samples to computer analysis and electronic data display may be computer controlled. Capillary electrophoresis is especially preferable for the sequencing of small pieces of DNA which might be present in limited amounts in a particular sample.

In another embodiment of the invention, the polynucleotides or modification thereof may be used in recombinant DNA molecules to direct expression of polypeptides for SHON, or modifications thereof, in appropriate host cells. Due to the inherent degeneracy of the genetic code, other DNA sequences which encode substantially the same or a functionally equivalent amino acid sequence may be produced, and these sequences may be used to clone and express polypeptides. The nucleotide sequences of the present invention can be engineered using methods generally known in the art in order to alter amino acid encoding sequences for a variety of reasons, including but not limited to, alterations which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, introduce mutations, and so forth.

In another embodiment of the invention, a natural, modified, or recombinant nucleic acid sequence encoding a polypeptide may be ligated to a heterologous sequence to encode a fusion protein. For example, it may be useful to encode a chimeric sequence that can be recognized by a commercially available antibody. A fusion protein may also be engineered to contain a cleavage site located between the polypeptide of the invention and the heterologous protein sequence, so that the polypeptide may be cleaved and purified away from the heterologous moiety.

In another embodiment, nucleotide sequences may be synthesized, in whole or in part, using chemical methods well known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the polypeptide itself may be produced using chemical methods to synthesize the amino acid sequence, or a modification thereof. For example, polypeptide synthesis can be performed using various solid-phase techniques (Roberge, J. Y. et al. (1995) Science 269:202-204; Merrifield J. (1963) J. Am. Chem. Soc. 85:2149-2154) and automated synthesis may be achieved, for example, using the ABI 431A Peptide Synthesizer (Perkin Elmer). Various fragments of polypeptides may be chemically synthesized separately and combined using chemical methods to produce the full length molecule.

The newly synthesized polypeptide may be isolated by preparative high performance liquid chromatography (e.g., Creighton, T. (1983) Proteins Structures and Molecular Principles, W H Freeman and Co., New York, N.Y.). The composition of the synthetic polypeptides may be confirmed by amino acid analysis or sequencing (e.g., the Edman degradation procedure; Creighton, supra). Additionally, the amino acid sequence of the polypeptide, or any part thereof, may be altered during direct synthesis and/or combined using chemical methods with sequences from other proteins, or any part thereof, to produce a modified molecule.

In order to express a biologically active polypeptides, the nucleotide sequences encoding the polypeptide or functional equivalents, may be inserted into appropriate expression vector, e.g., a vector which contains the necessary elements for the transcription and translation of the inserted coding sequence. Methods which are well known to those skilled in the art may be used to construct expression vectors containing sequences encoding the polypeptide and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y.; also, Sambrook, J. et al. (2000) Molecular Cloning, A Laboratory Manual, Cold Spring Harbor Laboratory Press, Plainview, N.Y., and Ausubel, F. M. et al. (1989) Current Protocols in Molecular Biology, John Wiley & Sons, New York, N.Y.

A variety of expression vector/host systems may be utilized to contain and express sequences encoding the polypeptides of the invention. These include, but are not limited to, microorganisms such as bacteria transformed with recombinant phage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems. For bacteria, useful plasmids include pET, pRSET, pTrcHis2, and pBAD plasmids from Invitrogen, pET and pCDF plasmids from Novagen, and Director™ plasmids from Sigma-Aldrich. In particular, E. coli can be used with the expression vector pET. The invention is not limited by the expression vector or host cell employed.

The “control elements” or “regulatory sequences” are those non-translated regions (e.g., enhancers, promoters, 5′ and 3′ untranslated regions) which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like may be used. The baculovirus polyhedrin promoter may be used in insect cells. Promoters or enhancers derived from the genomes of plant cells (e.g., heat shock, RUBISCO, and storage protein genes) or from plant viruses (e.g., viral promoters or leader sequences) may be cloned into the vector.

In bacterial systems, a number of expression vectors may be selected depending upon the use intended for the polypeptide. For example, when large quantities of polypeptide are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding a polypeptide may be ligated into the vector in frame with sequences for the amino-terminal Met and the subsequent 7 residues of β-galactosidase so that a hybrid protein is produced; pIN vectors (Van Heeke, G. and S. M. Schuster (1989) J. Biol. Chem. 264:5503-5509); and the like.

pGEX vectors (Promega, Madison, Wis.) and pQE vectors (Qiagen) may also be used to express the polypeptides as fusion proteins with glutathione S-transferase (GST), or HIS tag, respectively. In general, such fusion proteins are soluble and can easily be purified from lysed cells by adsorption to glutathione-agarose beads followed by elution in the presence of free glutathione. Proteins made in such systems may be designed to include heparin, thrombin, or factor Xa protease cleavage sites so that the cloned polypeptide of interest can be released from the GST moiety at will. In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

Specific initiation signals may also be used to achieve more efficient translation of sequences encoding the polypeptides of the invention. Such signals include the ATG initiation codon and adjacent sequences. In cases where sequences encoding a polypeptide, its initiation codon, and upstream sequences are inserted into the appropriate expression vector, no additional transcriptional or translational control signals may be needed. However, in cases where only coding sequence, or a modification thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell may be chosen for its ability to modulate the expression of the inserted sequences or to process the expressed polypeptide in the desired fashion. Such modifications of the sequence include, but are not limited to, acetylation, carboxylation, glycosylation, phosphorylation, lipidation, and acylation. Post-translational processing which cleaves a “prepro” form of the polypeptide may also be used to facilitate correct insertion, folding, and/or function. Different host cells which have specific cellular machinery and characteristic mechanisms for post-translational activities are available from the American Type Culture Collection (ATCC; Bethesda, Md.) and may be chosen to ensure the correct modification and processing of the sequence. Specific host cells include, but are not limited to, Rhodotorula, Aureobasidium, Saccharomyces, Sporobolomyces, Pseudomonas, Erwinia and Flavobacterium; or such other organisms as Escherichia, Lactobacillus, Bacillus, Streptomyces, and the like. Particular host cells include Escherichia coli, which is particularly suited for use with the present invention, Saccharomyces cerevisiae, Bacillus thuringiensis, Bacillus subtilis, Streptomyces lividans, and the like.

There are several techniques for introducing nucleic acids into eukaryotic cells cultured in vitro. These include chemical methods (Felgner et al., Proc. Natl. Acad. Sci., USA, 84:7413 7417 (1987); Bothwell et al., Methods for Cloning and Analysis of Eukaryotic Genes, Eds., Jones and Bartlett Publishers Inc., Boston, Mass. (1990), Ausubel et al., Short Protocols in Molecular Biology, John Wiley and Sons, New York, NY (1992); and Farhood, Annal. NY Acad. Sci., 716:23 34 (1994)), use of protoplasts (Bothwell, supra) or electrical pulses (Vatteroni et al., Mutn. Res., 291:163 169 (1993); Sabelnikov, Prog. Biophys. Mol. Biol., 62: 119 152 (1994); Bothwell et al., supra; and Ausubel et al., supra), use of attenuated viruses (Davis et al., J. Virol. 1996, 70(6), 3781 3787; Brinster et al. J. Gen. Virol. 2002, 83(Pt 2), 369 381; Moss, Dev. Biol. Stan., 82:55 63 (1994); and Bothwell et al., supra), as well as physical methods (Fynan et al., supra; Johnston et al., Meth. Cell Biol., 43(Pt A):353 365 (1994); Bothwell et al., supra; and Ausubel et al., supra).

A variety of protocols for detecting and measuring the expression of the polypeptides of the invention, using either polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay can be used with monoclonal antibodies reactive to two non-interfering epitopes on the polypeptide, but a competitive binding assay can also be used. These and other assays are described, among other places, in Hampton, R. et al. (1990; Serological Methods, a laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labelled hybridization or PCR probes for detecting sequences related to polynucleotides include oligolabelling, nick translation, end-labelling or PCR amplification using a labelled nucleotide. Alternatively, the sequences, or any modifications thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labelled nucleotides. These procedures may be conducted using a variety of commercially available kits Amersham Pharmacia Biotech, Promega, and US Biochemical. Suitable reporter molecules or labels, which may be used for ease of detection, include radionuclides, enzymes, fluorescent, chemiluminescent, or chromogenic agents as well as substrates, cofactors, inhibitors, magnetic particles, and the like.

Expression vectors or host cells transformed with expression vectors may be cultured under conditions suitable for the expression and recovery of the polypeptide from culture. The culture can comprise components for in vitro or in vivo expression. In vitro expression components include those for rabbit reticulocyte lysates, E. coli lysates, and wheat germ extracts, for example, Expressway™ or RiPs systems from Invitrogen, Genelator™ systems from iNtRON Biotechnology, EcoPro™ or STP3™ systems from Novagen, TNT® Quick Coupled systems from Promega, and EasyXpress systems from QIAGEN. The polypeptide produced from culture may be secreted or contained intracellularly depending on the sequence and/or the vector used. In particular aspects, expression vectors which encode a phage polypeptide can be designed to contain signal sequences which direct secretion of the polypeptide through a prokaryotic or eukaryotic cell membrane.

Other constructions may include an amino acid domain which will facilitate purification of the polypeptide. Such domains include, but are not limited to, metal chelating peptides such as histidine-tryptophan (e.g., 6X-HIS (SEQ ID NO: 11)) modules that allow purification on immobilized metals, protein A domains that allow purification on immobilized immunoglobulin, and the domain utilized in the FLAG® extension/affinity purification system (Immunex Corp., Seattle, Wash.). Useful epitope tags include 3X-FLAG®, HA, VSV-G, V5, HSV, GST, GFP, MBP, GAL4, and β-galactosidase. Useful plasmids include those comprising a biotin tag (e.g., PinPoint™ plasmids from Promega), calmodulin binding protein (e.g., pCAL plasmids from Stratagene), streptavidin binding peptide (e.g., InterPlay™ plasmids from Stratagene), a c-myc or FLAG® tag (e.g., Immunoprecipitation plasmids from Sigma-Aldrich), or a histidine tag (e.g., QIAExpress plasmids from QIAGEN).

To facilitate purification, expression vectors can include a cleavable linker sequences such as those specific for Factor Xa or enterokinase (Invitrogen, San Diego, Calif.). For example, the vector can include one or more linkers between the purification domain and the polypeptide. One such expression vector provides for expression of a fusion protein comprising a polypeptide of the invention and a nucleic acid encoding 6 histidine residues preceding a thioredoxin or an enterokinase cleavage site. The histidine residues facilitate purification on IMAC (immobilized metal ion affinity chromatography as described in Porath, J. et al. (1992) Prot. Exp. Purif. 3: 263-281) while the enterokinase cleavage site provides a means for purifying the polypeptide from the fusion protein. A discussion of vectors which contain fusion proteins is provided in Kroll, D. J. et al. (1993; DNA Cell Biol. 12:441-453).

Antibodies for SHON

The invention encompasses antibodies for SHON, for example, antibodies that bind to at least a portion of or a modified sequence thereof. In certain aspects of the invention, antibodies may be used to detect these ligands. Those of ordinary skill in the art to which the invention relates will recognize methods to generate antibody fragments. Antibody fragments of the invention can encompass a portion of one of the intact antibodies, generally the antigen binding or variable region of the antibody. However, by way of general example, fragments may be generated by proteolytic digestion of intact antibodies, or the fragments may be directly produced via recombinant nucleic acid technology.

The production of antibodies may be carried out according to standard methodology in the art. For example, in the case of the production of polyclonal antibodies the methodology of “Polyclonal Antibodies” described by Bean (Bean, 2000) may be used. Monoclonal antibodies and corresponding hybridomas may be prepared, for example, in accordance with the methodology of “Monoclonal Antibody Production” described by Stewart (Howard and Bethell, 2001), or of “Monocolonal Antibody Production Techniques and Applications” (Schook, 1987). Hybridomas may be subcloned, grown, and maintained using standard techniques in the art. For example, they may be grown and maintained in vitro in media such as DMEM or RPMI-1640. Alternatively, this may be done in vivo as ascites tumours in an animal of choice.

Antibodies of use in the invention may also be produced via standard recombinant techniques, see, e.g., “Recombinant monoclonal antibody technology” by Siegel (Siegel, 2002) and “Generation and screening of a modular human scFv expression library from multiple donors by Welschof et al. (Welschof et al., 2003). The inventors consider recombinant techniques to be a preferable means of producing antibodies on a commercial scale. Polynucleotides encoding an antibody may be readily identified on the basis of the amino acid sequence of the antibody, the genetic code, and the understood degeneracy therein. Polynucleotides encoding antibodies may be isolated from hybridoma cells, for example, and subsequently characterized using procedures standard in the art. For example, a polynucleotide probe may be designed based on the amino acid sequence of a portion of an antibody and then used to isolate genes encoding the heavy and/or light chains of the antibody. Alternatively, polynucleotides may be generated by standard chemical synthesis methodology, for example, using phosphoramidite and solid phase chemistry. The amino acid sequence of an antibody of the invention may be determined using standard methodology; for example, Edman degradation and HPLC or mass spectroscopy analysis, may be used.

In a preferred aspect, the invention encompasses three transcript variants termed SHONa, SHONb and SHONc, deposited on the 13 of Oct. 2012 with GenBank Accession Nos. JX965369, JX965370 and JX965371, respectively.

We have demonstrated that SHON expression promotes survival, migration, and invasion of carcinoma cells, in addition to anchorage-independent growth and colony formation (i.e., oncogenicity), and that these activities can be effectively inhibited by reducing the expression levels of SHON via siRNA or activity via antibodies. Therefore, we have shown SHON, affects cancer cell apoptosis, migration, and invasion, as well as anchorage-independent growth and colony formation (i.e., oncogenicity). As such, inhibition of these biological functions can be used to dramatically limit the onset, progression, metastasis, and recurrence of cancer, especially breast cancer, colon cancer, prostate cancer, endometrial cancer, lung cancer, stomach cancer, liver cancer, ovarian cancer, or another cancer.

The data disclosed herein demonstrates that SHON antibodies can effectively inhibit cell invasion and anchorage-independent growth, two important biological activities in cancer growth and metastasis, in both mammary carcinoma MCF-7 cells. As such, SHON antibodies represent ideal new reagents for cancer treatment and diagnosis, particularly for breast cancer, colon cancer, prostate cancer, endometrial cancer, lung cancer, stomach cancer, liver cancer, ovarian cancer, or another cancer. Antibodies that inhibit the biological activity and/or levels of SHON can be used to inhibit cell proliferation, cell survival, cell motility, and/or oncogenicity, for example, for cancer cells. These antibody inhibitors can be administered in conjunction with other agents, for example, chemical compounds (e.g., small molecules), antagonists, other antibodies, and iRNAs. Similarly, the antibodies or antibody fragments can be used to determine levels for SHON, and detect a cancerous condition, e.g., cancer onset, presence, progression, metastasis, or recurrence.

The data disclosed herein demonstrates that SHON antibodies directed against SHON can be used in diagnostic applications, for example, predicting the response of a patient to endocrine therapy, which comprises obtaining a sample from the patient and determining the expression of a SHON polypeptide in the sample, wherein an expression of SHON polypeptide in the sample indicates that the patient is endocrine therapy-responsive. In another aspect, this invention provides a method of providing a prognosis of disease-free survival of a tumour, which comprises obtaining a sample from the patient and determining the expression of a SHON polypeptide in the sample, wherein an expression of SHON polypeptide in the sample indicates the good prognosis of a prolonged disease-free survival.

In another aspect, this invention provides a method of predicting the propensity for distant metastatic spread of a tumour, which comprises obtaining a sample from the patient and determining the expression of a SHON polypeptide in the sample, wherein an expression of SHON polypeptide in the sample indicates that the patient the prognosis of a prolonged distant metastasis-free survival. In the case of diagnostic procedures, it is not necessary for the antibody to have inhibitor activity or decrease expression levels of the ligand. Although, as may be useful for certain applications, antibodies may be modified by labelling with a compound which provides a detectable signal; for example, enzymes, fluorescent agents, and radioisotopes can be used. Those of general skill in the art to which the invention relates will readily identify such suitable labelling systems. Additionally, antibodies may be used as carriers, for example to carry toxins, radionucleotides, isotopes, genes, or other therapeutic molecules to cells or tissues to aid in therapy. Persons of ordinary skill in the art will readily appreciate methods for determining the efficacy of an antibody in preventing, decreasing, or inhibiting cell proliferation, cell survival, cell motility, and/or oncogenicity. However, by way of example, the methodology described elsewhere herein, including one or more of the assays referred to in the examples section, may be used.

As will be appreciated, the antibodies, or antibody fragments, or modifications thereof may be used for the general purposes of detection and purification of SHON. The ligand may be from a natural or artificial source, such as a cell culture. Preferably, the ligand is of human origin. Additionally, as may be useful for certain applications, antibodies may be modified by labelling with a compound which provides a detectable signal. For example, enzymes, fluorescent agents, and radioisotopes can be used. Those of general skill in the art to which the invention relates will readily identify such suitable labelling systems. Thus, in addition to therapeutic use of antibodies directed against ligands, the antibodies may find use in purification of the ligands or in diagnostic applications. For example, antibodies immobilized on a solid phase would aid in purification and/or quantitation of the level of ligand in a sample. Those of ordinary skill in the art to which the invention relates will appreciate techniques by which this may be done. However, by way of example, affinity chromatography using antibodies, antibody fragments, or modifications may be used immobilized on a chromatographic support. In the case of diagnostic and purification procedures, it is not necessary for the antibody to have inhibitory activity.

It will be appreciated that ELISA or similar assays may incorporate both direct and indirect detection means, and that an antibody of the invention, or antibody fragment, or modification thereof, may be used as either capture or detection antibodies. As will be appreciated, one or more of the antibodies of the invention may be used in a single assay. For example, where two antibodies of the invention do not recognize the same antigenic determinant on SHON, one antibody may be used as a capture antibody and the other antibody may be used as a detection antibody. Alternatively, the antibodies of the invention can be used in combination with previously identified antibodies to the ligands. As will be appreciated by persons of ordinary skill in the art, the detection antibody used in an ELISA may be conjugated to a detectable label as herein described. In addition to ELISA, other useful assays include western blots, radioimmunoassays, immunoprecipitation assays, immunocytochemistry, immunohistochemistry, flow cytometry, Luminex® assays, and cytometric bead arrays.

Information of use in diagnosing or generally monitoring the status of a subject may be gained by making a direct comparison of the level of SHON in a test sample, with that of a determined base level or standard. For example, the average serum level of a ligand for a normal subject can be determined (i.e., a subject known not to present a medical disorder as described herein). These concentrations may be used as base levels, with a result above this range being indicative of a medical disorder. Preferably, the level necessary to be indicative of a disorder is a statistically significant increase of those ranges identified as normal. However, even where there is no statistically significant increase, results obtained may provide valuable information about the status of a subject. It should be appreciated that the normal ranges of ligand may differ in different body fluids and tissues. Similarly, normal levels of localized ligand may fall outside the range for normal levels in serum.

It should be appreciated that diagnosis or general determination of a subject's status may be made by comparing the level of SHON present in a test sample against a database of results obtained from a range of other subjects. Instead of utilizing a standard or base level concentration of ligand obtained from a number of normal subjects, the base level concentration may be determined from a single subject during a period when they were known not to present a medical disorder, or during a period of an active medical disorder. This may be particularly applicable to cases of on going and/or intermittent disease events or disorders where constant monitoring of the subjects status is required. For example, a base level may be determined during a period of remission from the disorder and the diagnostic procedure carried out at various times thereafter to assess status. This may provide valuable information pertaining to progression of a disorder, or help in assessing whether treatment of the disorder is proving successful.

In one aspect of the invention, the antibodies of the invention can be used for immunohistochemistry-based applications. For example, antibody staining can be used for the diagnosis of abnormal cells, such as those found in tumours, or for the characterization of particular cellular events such as cell proliferation or cell death, or for evaluating the localization and differential expression of proteins, e.g., SHON, in biological tissue. For immunohistochemistry, the antibody-ligand interaction can be visualized by various means. In particular, the antibody can be conjugated to an enzyme, such as peroxidase, which can catalyze a color-producing reaction. Alternatively, the antibody can be tagged to a fluorophore, such as FITC, rhodamine, Texas Red, Alexa Fluor®, or DyLight™ Fluor, which can be viewed by immunofluorescence microscopy. Fluorophor tagging is particularly useful for confocal laser scanning microscopy, which is highly sensitive and can be used to visualize interactions between multiple proteins. As another approach, secondary antibodies can be used to amplify the antibody signal. The secondary antibodies can be conjugated, for example, to biotin or a reporter enzyme such as alkaline phosphatase or horseradish peroxidase, or to fluorescent agents as described in detail herein. For immunohistochemistry, any cells or tissues from a biopsy can be used, or any biological sample as described herein.

As described herein, antibodies produced in accordance with the invention may find particular use as therapeutic agents, for example, for preventing, decreasing, or inhibiting cell proliferation, cell survival, cell motility, and/or oncogenicity. In one broad embodiment the invention provides a method of blocking the interaction of at least one ligand with one or more receptors, or more broadly, blocking the interaction of a ligand with a binding agent, the method comprising contacting the antibody, antibody fragment, or modification thereof in accordance with the invention. This method may be conducted in vivo or in vitro. Persons of ordinary skill in the art will readily appreciate methods for determining the efficacy of an antibody in preventing, decreasing, or inhibiting cell proliferation, cell survival, or cell motility. However, by way of example, the methodology described elsewhere herein, including one or more of the assays referred to in the “Examples” section, may be used. Additionally, the antibodies of the invention may be used as carriers, for example to carry toxins, radionucleotides, isotopes, genes, or other therapeutic molecules to cells or tissues to aid in therapy.

The invention further encompasses immunotoxins comprising an SHON antibody or antibody fragment, which is linked to a toxic agent. Such agents include pharmacologic toxins that can be conjugated to an antibody and delivered in an active form to a cell, wherein they will exert a significant deleterious effect. The preparation of immunotoxins is, in general, well known in the art (see, e.g., U.S. Pat. No. 4,340,535, incorporated herein by reference). Exemplary toxic agents include chemotherapeutic agents, radioisotopes as well as cytotoxins. Example of chemotherapeutic agents are hormones such as steroids; antimetabolites such as cytosine arabinoside, fluorouracil, methotrexate or aminopterin; anthracycline; mitomycin C; vinca alkaloids; demecolcine; etoposide; mithramycin; or alkylating agents such as chlorambucil or melphalan. Useful immunotoxins include plant-, fungal- or bacterial-derived toxins, such as an A chain toxin, a ribosome inactivating protein, alpha-sarcin, aspergillin, restirictocin, a ribonuclease, diphtheria toxin or pseudomonas exotoxin, to mention just a few examples.

The use of toxin-antibody constructs is well known in the art of immunotoxins, as is their attachment to antibodies. Of course, combinations of the various toxins could also be coupled to one antibody molecule, thereby accommodating variable or even enhanced cytotoxicity. One particular type of toxin for attachment to antibodies is ricin, with deglycosylated ricin A chain being particularly preferred. Various recombinant or genetically engineered forms of the ricin molecule are known to those of skill in the art, all of which may be employed in accordance with the present invention. Deglycosylated ricin A chain (dgA) is useful because of its extreme potency, longer half-life, and because it is economically feasible to manufacture it a clinical grade and scale. Truncated ricin A chain, from which the N-terminal amino acids have been removed by Nagarase (Sigma), also may be employed. While IgG based immunotoxins will typically exhibit better binding capability and slower blood clearance than their Fab′ counterparts, Fab′ fragment-based immunotoxins will generally exhibit better tissue penetrating capability as compared to IgG based immunotoxins.

Methods of Treatment

While the inventors' primary studies have involved breast cancer cells, SHON is predicted to also act in the small intestine and kidney; and in the heart, prostate, uterus, normal colon, stomach, skin, trachea, brain, cerebellum, foetal brain, spinal cord, placenta, adipose tissue, cartilage; and also in the thymus. Specifically, SHON is predicted to act in breast cancer, colon cancer, prostate cancer, endometrial cancer, lung cancer, stomach cancer, liver cancer, and ovarian cancer, among others. Accordingly, the inventors contemplate the predicting value being applicable to the treatment of a variety of disorders characterized by increased or aberrant cell proliferation, cell survival, cell motility, and/or oncogenicity.

In one embodiment, the disorder is an epithelial tumour of the breast, prostate, colon, lung, pancreas, endometrium, or ovary, or squamous cell carcinoma, or a melanoma, or a renal cancer or tumour. Regarding breast cancers, these can include epithelial tumours (e.g., from cells lining ducts or lobules) or nonepithelial tumours (e.g., from the supporting stroma), such as angiosarcomas, primary stromal sarcomas, and phyllodes tumour. Breast cancers can also include carcinomas, for example, carcinomas in situ, as well as invasive cancers. Carcinoma in situ includes proliferation of cancer cells within ducts or lobules and without invasion of stromal tissue. Lobular carcinoma in situ (LCIS) includes nonpalpable lesions which can indicate increased risk of subsequent invasive carcinoma in either breast. In breast cancer, invasive carcinoma generally comprises adenocarcinoma, with most comprising infiltrating ductal type carcinoma and the remainder comprising infiltrating lobular carcinoma. Rare forms of breast cancer include medullary, mucinous, and tubular carcinomas. Breast cancer disorders also include Paget's disease of the nipple, and metastatic breast cancer.

Regarding colon cancer, this can generally include cancer of the colon, rectum, and/or anus, and especially, adenocarcinomas, and also carcinomas (e.g., squamous cloacogenic carcinomas), melanomas, lymphomas, and sarcomas. Epidermoid (nonkeratinizing squamous cell or basaloid) carcinomas are also included. The colon cancer may be associated with particular types of polyps or other lesions, for example, tubular adenomas, tubulovillous adenomas (e.g., villoglandular polyps), villous (e.g., papillary) adenomas (with or without adenocarcinoma), hyperplastic polyps, hamartomas, juvenile polyps, polypoid carcinomas, pseudopolyps, lipomas, or leiomyomas. The cancer may be associated with familial polyposis and related conditions such as Gardner's syndrome or Peutz-Jeghers syndrome. The cancer may be associated, for example, with chronic fistulas, irradiated anal skin, leukoplakia, lymphogranuloma venereum, Bowen's disease (intraepithelial carcinoma), condyloma acuminatum, or human papillomavirus. In other aspects, the cancer may be associated with basal cell carcinoma, extramammary Paget's disease, cloacogenic carcinoma, or malignant melanoma.

Regarding endometrial cancers, these can include adenocarcinomas and also papillary serous, clear cell, squamous, and mucinous carcinoma. Also included are precancerous conditions such as endometrial hyperplasia. The endometrial cancer may be associated with one or more of obesity, polycystic ovarian syndrome, nulliparity, late menopause, estrogen-producing tumors, anovulation (ovulatory dysfunction), and estrogen therapy without progesterone and hereditary nonpolyposis colorectal cancer (HNPCC) syndrome.

Regarding ovarian cancers, these commonly originate in the epithelium and can include cystadenocarcinomas as well as Brenner tumours, clear cell carcinomas, endometrioid carcinomas, mucinous carcinomas, transitional cell carcinomas, in addition to unclassified carcinomas. For ovarian cancers originating from germ cells, these can include choriocarcinomas, dysgerminomas, embryonal carcinomas, endodermal sinus tumors, immature teratomas, and polyembryomas. For ovarian cancers originating from sex cord and stromal cells, these include granulosa-theca cell tumors and Sertoli-Leydig cell tumors. For ovarian cancers originating from metastases, these include those from breast cancers and cancers of the GI tract, as well as others.

Persons of general skill in the art to which the invention relates may readily appreciate alternative types of disorder which the invention may be applicable, especially having regard to the expression of SHON provided herein. In addition, it will be appreciated by those of general skill in the art to which the invention relates, having regard to the nature of the invention and the results reported herein, that the present invention is applicable to a variety of different animals. Accordingly, the diagnostic cell treatment methods can apply to any animal of interest. In particular, the invention is applicable to mammals, more particularly humans.

Persons of ordinary skill in the art to which the invention relates will appreciate various means and agents for use in detecting SHON. By way of example, antibodies directed against SHON, or functional modifications of such antibodies may be used. Exemplary agents are described in detail herein. Those agents of use in the invention will preferably exhibit one or more of the following characteristics: 1) the ability to detect SHON peptides; 2) the ability to detect SHON transcripts.

As shown herein, SHON is encoded as a cellular factor that is expressed in certain cancer cells, but not by at least one subset of normal adult cells. Therefore, SHON can be considered a tumour-associated antigen. Several approaches can be used to detect these ligands based on differences in expression and access in normal and cancer cells (reviewed, generally, in Paul, Fundamental Immunology, 1999, Lippincott-Raven Publishers, Philadelphia, Pa., Chapter 37). Cancer cells are likely to express tumour-associated antigens at much higher levels and such differences in expression levels between normal and cancer cells can be exploited therapeutically (see, e.g., Brown J P, et al., Quantitative analysis of melanoma-associated antigen p97 in normal and neoplastic tissues. Proc Natl Acad Sci USA 1981; 78:539-543).

The assays described herein after under the heading “Examples” may be used to determine the suitability of an agent in accordance with the invention. Specifically, RT-PCR and Northern blot analysis can be used to detect expression at the mRNA level, and Western blotting and direct or indirect immunostaining can be used to detect the expression at the protein level. To detect activity, cell-based assays for cell proliferation, cell survival, cell motility, and/or oncogenicity can be used. Regarding inhibition of metastasis, an in vivo assay may be used, as described, for example, in Fidler, I. J. (1973) Nat. New Biol. 242, 148-149; and Price J. E. The biology of cancer metastasis. Prog. Clin. Biol. Res., 354A: 237-255, 1990, or Kerbel R. S. What is the optimal rodent model for anti-tumor drug testing? Cancer Metastasis Rev., 17: 301-304, 1998; Killion J. J., Radinsky R., Fidler I. J. Orthotopic models are necessary to predict therapy of transplantable tumors in mice. Cancer Metastasis Rev., 17: 279-284, 1998; and Price J. E. Analyzing the metastatic phenotype. J. Cell. Biochem., 56: 16-22, 1994.

Kits for Treatment or Diagnosis

The agents and compositions of the invention may be used in kits suitable for detecting SHON, or for the treatment of a disorder as defined herein. The agents and compositions may also be used in diagnostic kits. Kits can comprise at least one agent of the invention in a suitable container. The kit may include an SHON antibody or antibody fragment, a pair of DNA primers which specifically bind to the SHON nucleotide sequence or a modification thereof. The kit may further include one or more ancillary reagents suitable for detecting the presence of a complex between the antibody or antibody fragment and SHON or portion thereof These can be provided in further separate containers as may be necessary for a particular application. Where a secondary antibody capable of binding to the primary antibody is employed, such secondary antibody can be provided in the kit, for instance in a separate vial or container. The secondary antibody, if present, is typically labelled, and may be formulated in an analogous manner with the antibody formulations described herein.

For a kit, the antibody compositions of the present invention may be provided either alone or in combination with additional antibodies specific for other epitopes. The antibody or antibody fragment may be labelled or unlabelled, and may be provided with adjunct ingredients, e.g., buffers, such as Tris, phosphate and carbonate, stabilizers, excipients, biocides and/or inert proteins, such as bovine serum albumin. Generally these adjunct materials will be present in less than about 5% weight based on the amount of active antibody, and usually will be present in a total amount of at least about 0.001% weight based on antibody concentration. The antibodies or antibody fragments can be provided as a lyophilized mixture with the adjunct ingredients, or the adjunct ingredients can be separately provided for combination by the user.

In a particular aspect, the kit can include, in an amount sufficient for at least one diagnostic assay, an antibody of the present invention or a fragment thereof as a separately packaged reagent. The antibody or antibody fragment can be provided as reagent in combination with a solid phase support or bead. The kit can be directed to the isolation of SHON polypeptides or peptides or cells expressing SHON. In specific aspects, the kit can be directed to FACS analysis. The kit can comprise a hybridoma cell line as disclosed herein, and allow production of an SHON antibody. In particular aspects, a cell culture medium for said hybridoma cell line can be included.

In the case of therapeutics, the antibodies and antibody fragments may be formulated suitable for direct administration to a subject for example, as pharmaceutical compositions. Alternatively, the kit may comprise one or more agents in one container and pharmaceutical diluents, carriers and/or excipients in another; the contents of each container being mixed together prior to administration. Any container suitable for storing and/or administering an agent or composition may be used in a kit of the invention. Suitable containers will be appreciated by persons skilled in the art. By way of example, such containers include vials and syringes. The containers may be suitably sterilized and hermetically sealed. Further, kits of the invention can also comprise instructions for the use and administration of the components of the kit.

Diagnostic Methods and Compositions

In one embodiment, the invention relates to use of one or more reagents of the invention in a method of detecting SHON in a disorder associated with SHON. Specific disorders can include, for example, cancer (breast cancer, colon cancer, prostate cancer, endometrial cancer, lung cancer, stomach cancer, liver cancer, ovarian cancer, for example) and endometriosis. Exemplary disorders are described in detail herein.

In accordance with the invention, antibodies which specifically bind SHON may be used for the diagnosis of conditions or disorders characterized by altered expression, or in assays to monitor patients being treated. The antibodies useful for diagnostic purposes may be prepared in the same manner as those described above. Diagnostic assays include methods which utilize the antibody and a label to detect the corresponding peptide or polypeptide in human body fluids or extracts of cells or tissues. The antibodies may be used with or without modification, and may be labelled by joining them, either covalently or non-covalently, with a reporter molecule. A wide variety of reporter molecules which are known in the art may be used, several of which are described herein.

A variety of protocols, including ELISA, RIA, and FACS, are known in the art and provide a basis for diagnosing altered or abnormal levels of expression for SHON. Normal or standard values for expression are established by combining body fluids or cell extracts taken from normal mammalian subjects, preferably human, with antibody under conditions suitable for complex formation. The amount of standard complex formation may be quantified by various methods, but preferably by photometric means. Quantities expressed in subject, control, and disease, samples from biopsied tissues are compared with the standard values. Deviation between standard and subject values establishes the parameters for diagnosing disorders.

The antibodies or antibody fragments may be used to detect and quantitate expression in biopsied tissues in which expression may be correlated with disorders. The diagnostic assay may be used to distinguish between absence, presence, and altered expression, and to monitor regulation of levels during therapeutic intervention. In one aspect, nucleic acid hybridization can be performed with antibodies or antibody fragments which are capable of detecting polypeptide sequences for SHON, or fragments, or resultant modifications. For example, the antibodies or antibody fragments of the subject invention may recognize an amino acid sequence of the invention, e.g., one or more of SEQ ID NO: 2, 5 and 6, or modified sequences thereof

Antibodies for SHON may be used for the diagnosis of disorders which are associated with either increased or decreased expression. The antibodies or antibody fragments may be used in qualitative or quantitative methods are well known in the art. In a particular aspect, the antibodies for SHON may be useful in assays that detect activation or induction of various cancers, particularly those mentioned above. The antibodies or antibody fragments may be labelled by standard methods, and added to a fluid or tissue sample from a patient under conditions suitable for the formation of binding complexes. After a suitable incubation period, the sample is washed and the signal is quantitated and compared with a standard value. If the amount of signal in the biopsied or extracted sample is significantly altered from that of a comparable control sample, the presence of altered levels of amino acid sequences in the sample indicates the presence of the associated disorder. Such assays may also be used to evaluate the efficacy of a particular therapeutic treatment regimen in animal studies, in clinical trials, or in monitoring the treatment of an individual patient.

In order to provide a basis for the diagnosis of a disorder associated with expression of SHON, a normal or standard profile for expression is established. This may be accomplished by combining body fluids or cell extracts taken from normal subjects, either animal or human, with an antibody or antibody fragment, or a modification thereof, for SHON, under conditions suitable for binding. Standard binding may be quantified by comparing the values obtained from normal subjects with those from an experiment where a known amount of a substantially purified polypeptide is used. Standard values obtained from normal samples may be compared with values obtained from samples from patients who are symptomatic for a disorder. Deviation between standard and subject values is used to establish the presence of the disorder.

Methods which may also be used to quantitate the expression of SHON include radiolabelling antibodies, binding with a control polypeptide, and standard curves onto which the experimental results are interpolated. The speed of quantitation of multiple samples may be accelerated by running the assay in an ELISA format where the sequence of interest is presented in various dilutions and a spectrophotometric or colorimetric response gives rapid quantitation.

In further embodiments, antibodies, antibody fragments, or modifications thereof as described herein may be used as reagents in a microarray. The microarray can be used to monitor the expression level of large number of samples simultaneously and to develop and monitor the activities of therapeutic agents. The microarrays may be prepared and used according to the methods known in the art. The microarray substrate may be paper, nylon or any other type of membrane, filter, chip, plate such as a microtiter plate, glass slide, or any other suitable solid support. In one aspect, a gridded array analogous to a dot or slot blot may be used to link samples to the surface of a substrate. In yet another aspect, an array may be produced by hand or by using available devices, materials, and machines (including multichannel pipettors or robotic instruments) and may include about 8, 24, 96, 384, 1536 or 6144 samples, or any other multiple from 2 to 1,000,000, which lends itself to the efficient use of commercially available instrumentation.

In order to conduct sample analysis using the microarrays, polypeptides may be extracted from a biological sample. The biological samples may be obtained from any bodily fluid (e.g., blood, urine, saliva, phlegm, gastric juices, etc.), cultured cells, biopsies, or other tissue preparations. Labelled antibodies or antibody fragments may be incubated with the microarray so that they bind to the polypeptides of the microarray. Incubation conditions can be adjusted so that binding occurs with specificity. After removal of unbound antibodies, a scanner can be used to determine the levels and patterns of label. The scanned images are examined to determine the relative abundance of a polypeptide sequence on the microarray. A detection system may be used to measure the absence, presence, and amount of binding for a number of distinct sequences simultaneously.

In another embodiment, the antibodies of the invention may be used for immunohistochemistry-based applications. In accordance with standard immunohistochemistry techniques, thin slices (e.g., about 4-40 μm) can be taken from the tissue of interest, or the tissue can be used whole. The slicing can be accomplished through the use of a microtome, and slices can be mounted on slides. The tissue can then be treated to rupture the cell membranes, e.g., using detergent such as Triton X-100. Additional unmasking steps can also be used, as well as blocking steps to minimize non-specific binding. For direct immunohistochemistry, the labelled antibody of interest (e.g. FITC conjugated antiserum) is used to bind the antigen in the tissue sections. For indirect immunohistochemistry, an unlabelled primary antibody is used to bind to the tissue antigen, and a labelled secondary antibody is used to react with the primary antibody.

The antibodies and antibody fragments may also be used as in vivo diagnostic agents to provide an image of cancer cells (e.g., tumours) or respective metastases. Various diagnostic methods such as magnetic resonance imaging (MRI), X-ray imaging, computerized emission tomography and similar technologies may be employed. In this type of imaging, the antibody portion used will generally bind to the cancer marker, such as SHON, and the imaging agent will be an agent detectable upon imaging, such as a paramagnetic, radioactive or fluorescent molecule. Many appropriate imaging agents are known in the art, as are methods for their attachment to antibodies (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4,472,509). Antibodies also may be reacted with an enzyme in the presence of a coupling agent such as glutaraldehyde or periodate. Conjugates with fluorescein markers are prepared in the presence of these coupling agents or by reaction with an isothiocyanate.

In the case of paramagnetic ions, exemplary agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III). In the case of radioactive isotopes for diagnostic application, useful agents include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵I is commonly used, and technicium^{99m} and indium¹¹¹ are also often used due to their low energy and suitability for long range detection. Elements particularly useful in MRI include the nuclear magnetic spin-resonance isotopes ¹⁵⁷Gd, ⁵⁵Mn, ¹⁶²Dy, ⁵²Cr, and ⁵⁶Fe , with gadolinium often being preferred.

Radioactively labelled antibodies and antibody fragments of the present invention may be produced according to well-known methods in the art. For instance, antibodies can be iodinated by contact with sodium or potassium iodide and a chemical oxidizing agent such as sodium hypochlorite, or an enzymatic oxidizing agent, such as lactoperoxidase. Antibodies according to the invention may be labelled with technetium^{99m} by ligand exchange process, for example, by reducing pertechnate with stannous solution, chelating the reduced technetium onto a Sephadex column and applying the antibody to this column or by direct labelling techniques, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) and ethylene diaminetetracetic acid (EDTA).

A factor to consider in selecting a radionuclide for in vivo diagnosis is that the half-life of a nuclide be long enough so that it is still detectable at the time of maximum uptake by the target, but short enough so that deleterious radiation upon the host, as well as background, is minimized. Ideally, a radionuclide used for in vivo imaging will lack a particulate emission, but produce a large number of photons in a 140 2000 keV range, which may be readily detected by conventional gamma cameras. Administration of the labelled antibody may be local or systemic and accomplished intravenously, intra-arterially, via the spinal fluid or the like. Administration also may be intradermal or intracavitary, depending upon the body site under examination. After a sufficient time has lapsed for the labelled antibody or fragment to bind to the diseased tissue, in this case cancer tissue, for example 30 min to 48 h, the area of the subject under investigation is then examined by the imaging technique. MRI, SPECT, planar scintillation imaging and other emerging imaging techniques may all be used. The distribution of the bound radioactive isotope and its increase or decrease with time is monitored and recorded. By comparing the results with data obtained from studies of clinically normal individuals, the presence and extent of the diseased tissue can be determined. The exact imaging protocol will necessarily vary depending upon factors specific to the patient, and depending upon the body site under examination, method of administration, type of label used and the like.

In further embodiments there is provided a reagent comprising at least one pair of DNA primers which specifically bind to the SHON nucleotide sequence.

In additional embodiments, the antibodies or antibody fragments for SHON may be used in any molecular biology techniques that have yet to be developed, provided the new techniques rely on properties of antibodies that are currently known, including, but not limited to, such properties as binding specificity.

The invention is further elucidated with reference to the examples below.

### EXAMPLES

The examples described herein are for purposes of illustrating embodiments of the invention.

Other embodiments, methods, and types of analyses are within the scope of persons of ordinary skill in the molecular diagnostic arts and need not be described in detail herein. Other embodiments within the scope of the art are considered to be part of this invention.

### Example 1

### Materials and Methods

Cell Culture

The normal but immortalised human mammary epithelial cell line MCF10A as well as all the carcinoma cell lines were obtained from the American Type Culture Collection, including lung cancer (A549 and H1975), stomach cancer (AGS and MKN-45), prostate cancer (DU145, PC3 and LnCap), endometrial cancer (RL95-2 and AN3), breast cancer (MCF-7, T47D, BT474, BT459 and MDA-MB-231), and ovarian cancer (Ovca4). All were cultured in conditions as recommended, except that the MCF-7 cell line was cultured in RPMI 1640 medium supplemented with 10% heat-inactivated foetal bovine serum (FBS), 100 units/ml penicillin, 100 μg/ml streptomycin, and 2 mM L-glutamine in a humidified incubator in 5% CO₂ at 37° C.

Plasmid Constructs

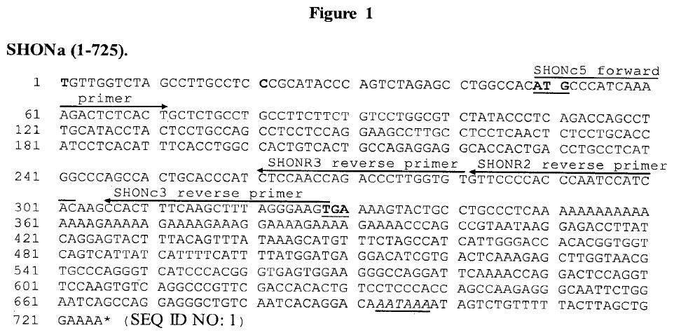

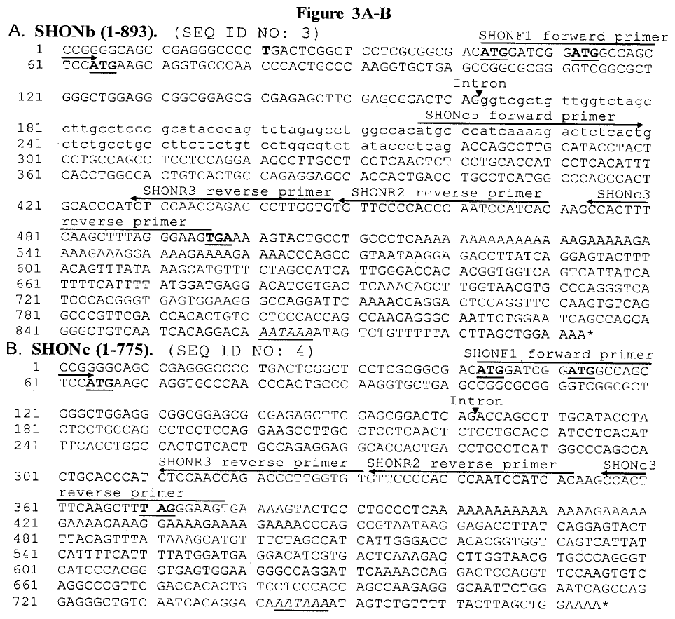

Mammalian expression plasmids—The coding sequence for full-length human SHON α (FIG. 1) and β (FIG. 3A) were cloned into the mammalian expression vector pIRESneo3 (Invitrogen, Life Technologies), designated pIRESneo3-SHONα and pIRESneo3-SHONβ, respectively. The SHONβ mutant expressing plasmid pIRESneo3-SHONβni, in which the potential proprotein convertase motif K⁶²R⁶³ was mutated into N⁶²I⁶³, was generated from the pIRESneo3-SHONβ by PCR-based site-directed mutagenesis. In some cases, a c-Myc epitope (YALEQKLISEEDL (SEQ ID NO: 12), polypeptide resulted from the linker is underlined), 6×HIS tag (SEQ ID NO: 11), or the EGFP (as coded by the pEGFP-C1 vector) was fused at the C-terminal of the proteins expressed from the plasmids using standard methods.

SHON siRNA vectors—To generate siRNA oligonucleotides targeting all three variants of SHON mRNA, the DNA sequence 5′-AATCCATCACAAGCCACTTTC-3′ (SEQ ID NO: 13) was selected (from all the 5 sequences tested) to construct an siRNA expression plasmid using the pSilencer 2.1-U6 hygro vector (Ambion) according to the manufacturer's protocol. The resultant vector was designated pSilencer-siRNA. BLAST search against the human genome sequence showed that only the SHON gene was targeted. The negative control siRNA plasmid (pSilencer-CK) encodes an siRNA which has no significant sequence similarity to human gene sequences (Ambion).

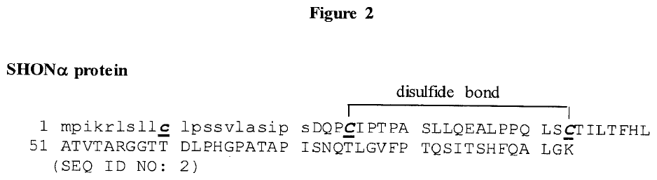

Bacterial expression plasmids—The cDNA of mature SHONa (FIG. 2) was cloned into the pGEX-4T1 vector (Amersham Biosciences, Piscataway, N.J., USA) to generate pGEX-4T1-SHONα for expression of glutathione-S-transferase (GST) recombinant proteins in bacteria. For production of recombinant HIS tagged mature SHONα, the cDNA of mature SHONα was cloned into the pQE30 vector (Qiagen) to generate the pQE30-SHONα vector.

Establishing Stable Cell Lines

MCF-7 cells were stably transfected with SHONα expression plasmid pIRESneo3-SHONα (designated MCF7-SHON) or the empty control pIRESneo3 vector (MCF7-Vec) as a control using Saint-Mix transfection reagent (Synvolux Therapeutics B. V., the Netherlands). Cell clones were selected by addition of G418 at 800 μg/ml (Bio-Rad Laboratories, Calif.) in the medium. Transfected cell lines were generated as pools of positive cell clones. MCF-7 cells were similarly stably transfected with SHON siRNA plasmid pSilencer-siRNA (MCF7-siRNA) or the negative siRNA control plasmid pSilencer-CK (MCF7-CK). siRNA stable cell clones were selected by addition of hygromycin to a concentration of 100 μg/ml in the medium. The overexpression of SHON or depletion of endogenous SHON in established stables were confirmed by RT-PCR and Western blotting.

Preparation of Total RNA

Total RNA was isolated from cultured cells with Trizol reagent (Invitrogen) at 1 ml/10 cm² according to the manufacturer's instructions. RNA was resuspended in diethyl pyrocarbonate (DEPC)-treated nuclease-free water. RNA samples were further treated with DNase I for 30 min at 37° C. The reaction was stopped by addition of 25 mM EDTA and incubation at 65° C. for 15 min. RNA samples were then purified by extraction in phenol/chloroform (pH 5.2, phenol:chloroform:isoamyl alcohol at 25:24:1) followed by an additional chloroform extraction and ethanol precipitation. Quantification and purity of the RNA was assessed by A₂₆₀/A₂₈₀ absorption, and RNA quality was assessed by agarose gel electrophoresis. RNA samples with ratios of A₂₆₀/A₂₈₀ greater than 1.6 were stored at −80° C. for further analysis.

Reverse Transcription-PCR

One-step reverse transcription (RT)-PCR kit (Qiagen) was used to determine the presence of an mRNA using gene-specific primers (see Table 1 for details). For RT-PCR, the following procedure was employed. To start, 1 μg of total RNA was diluted to a concentration of 0.1 μg/μl to minimize the variation of sample handling. This dilution was treated by DNase I for 15 min, followed by inactivation of DNase by adding EDTA to 5 mM and heating to 70° C. for 15 min. The DNase-treated RNA was then mixed with a master cocktail containing RT-PCR buffer, sense and antisense primers, dNTPs, RNase inhibitor, an enzyme mixture containing reverse transcriptase (Omniscript and Sensiscript) and HotStart Taq DNA polymerase at the concentrations recommended by the manufacturer to a final volume of 50 μl.

The temperature-cycle protocol included: 60 min at 50° C. for the reverse transcription reaction, followed by denaturation and activation of HotStart DNA polymerase for 15 min at 95° C., and PCR amplification for 20 sec at 95° C., 30 sec at 54-62° C., and 1 min at 72° C. for 30 cycles. A final extension for 5 min at 72° C. was performed at the end of the cycles. (3-actin was similarly amplified by RT-PCR using 0.2 μg of total RNA as an internal control. Ten microlitres of each of the RT-PCR product was fractionated on 1% agarose gels. The identity of RT-PCR product was confirmed by the size, restriction enzyme digestion, and DNA sequencing.

Immunoblotting

Mammalian cells were washed twice with ice-cold phosphate-buffered saline (PBS) and lysed at 4° C. in lysis buffer (20 mM Tris●HCl, pH 7.4, 150 mM NaCl, 1 mM EDTA, 1 mM EGTA, 1% Triton® X-100, 1% Nonidet P-40, 1 μg/ml protease inhibitor cocktail (GE Healthcare) and 0.1 mM PMSF). The lysates were next sonicated and then cleared by centrifugation at 15,000×g for 15 min at 4° C. SDS-polyacrylamide gel electrophoresis (PAGE) sample buffer (50 mM Tris●HCl, pH 6.8; 2% SDS; 2% β-mercaptoethanol, and bromophenol blue) was added to each sample and the samples were boiled for 5 min.

E. coli bacteria cells were directly lysed using 2×SDS-PAGE sample buffer (50 mM Tris●HCl, pH 6.8; 2% SDS; 2% β-mercaptoethanol, and bromophenol blue) and the samples were then boiled for 5 min before loading.

Samples were subjected to discontinuous SDS-PAGE with a 15% resolving gel and transferred to nitrocellulose membranes (Hybond™ C-extra) using standard electroblotting procedures. Membranes were blocked with 5% non-fat dry milk in PBS with 0.1% Tween® 20 (PBST) for 1 h at room temperature. The blots were then immunolabelled with primary antibodies in PBST containing 1% non-fat dry milk at 4° C. overnight.

After incubation with appropriate secondary antibodies at room temperature, immunolabelling was detected by ECL plus™ chemiluminescence as described by the manufacturer (GE Healthcare). Blots were stripped and reprobed with monoclonal antibody against β-actin to ensure equal loading of the cell lysate proteins. Blots were stripped by incubation for 30 min at 50° C. in a solution containing 62.5 mM Tris●HCl, pH 6.7; 2% SDS; and 0.7% β-mercaptoethanol. Blots were then washed for 30 min with several changes of PBST at room temperature. Efficacy of stripping was determined by re-exposure of the membranes to ECL plus™. Thereafter, blots were re-blocked and immunolabelled as described above.

Production of Recombinant Human SHONα Protein in Bacteria

Recombinant glutathione-S-transferase (GST) tagged SHONα fusion protein (GST-SHONα) was produced by transformation of the pGEX-4T1-SHONα plasmid into E. coli BL21-DE3-LysS pLysS competent cells. Isopropyl β-D-1-thiogalactopyranoside (IPTG) (50 μM) was used to induce the expression. The recombinant protein was purified by Glutathione Sepharose 4B beads (GE Healthcare, Little Chalfont, UK) under native conditions. Dialysed GST-SHONα protein was used for rabbit immunizations. The yield of purified protein was estimated by Bradford's assay.

A HIS tagged SHONα protein (HIS-SHONα) was also produced in E. coli M15 strain by transforming the pQE30-SHONα plasmid. 200 μM of IPTG was used to induce HIS-SHONα expression and Nickel beads (Qiagen) were used to purify HIS-SHONα under native conditions. Serial concentrations of imidazole were tested to elute the recombinant protein. Each elute was run on a SDS PAGE and Western blotted to test the presence and purity of HIS-SHONα protein.

Production of Rabbit Polyclonal Antibodies

Polyclonal antisera against human SHONα was generated using subcutaneous and intramuscular injections of the immunogen into rabbits as previously described (Bean, 2000). Briefly, each rabbit was injected with 400 μg of the purified GST-SHONα antigen with complete Freunds' adjuvant (Sigma-Aldrich, Mo., USA), followed by an injection with 200 μg of antigen with incomplete Freunds' adjuvant (Sigma-Aldrich, Mo., USA) every 2-3 weeks.

SHONα antibodies were affinity-purified from the antisera using standard methodology sequentially, first by using GST protein covalently bound to Glutathione Sepharose 4B beads to remove anti-GST antibodies, and followed by using GST-SHONα protein covalently bound to Glutathione Sepharose 4B beads.

Determination of the Full mRNA Sequence of SHON by Rapid Amplification of 5′ Complementary DNA Ends (5′ RACE) 5′RACE (Frohman et al., 1988) was carried out to determine the 5′upstream sequence of SHON/PIKR2786/UNQ2786 represented by the EST clone (GenBank accession number AY358103) (Clark et al., 2003). Briefly, a SHON specific antisense primer SHONc3 (5′-ACTTCCCTAAAGCTTGAAAGTGG-3′ (SEQ ID NO: 14), FIG. 1) was used to synthesize cDNA from the total RNA isolated from mammary carcinoma MCF-7 cells using M-MuLV reverse transcriptase (New England Biolabs). The synthesised cDNA was purified and polyadenylated using terminal deoxynucleotidyl transferase (TdT) (New England Biolabs) in the presence of dATP. The polyadenylated cDNA was first amplified by PCR using Vent DNA polymerase with the SHONc3 primer and a sense primer dT17TAG (5′-CCGGACGCGTCGACTAGTACTTTTTTTTTTTTTTTTT-3′ (SEQ ID NO: 15)) for 40 cycles each consisting of 94° C., 1 min; 60° C., 1 min; 72° C., 1 min, with a final 5 min extension at 72° C. The PCR reaction was diluted 1,000-fold, and a second and third round of PCR was carried out using the dT17TAG sense primer and two SHON internal antisense primers, SHONR2 (5′-GTGATGGATTGGGTGGGGAAC-3′ (SEQ ID NO: 16)) and SHONR3 (5′-ACACCAAGGGTCTGGTTGGAG-3′ (SEQ ID NO: 17)), respectively (FIG. 1). The PCR product from the third round of PCR was directly cloned into the pPCR-Script Amp SK(+) at the Srf I restriction site. The sequence of the cloned inserts was determined by DNA sequencing.

Determination of SHON Transcripts by RT-PCR

RT-PCR was used to determine the expression of SHON mRNA in MCF-7 cells. Since transcripts b and c had a large intron (see Results section below), it was easy to detect their mRNA expression. However, transcript a was difficult to detect because it is intronless and it was therefore not possible to design primer pairs that only specific to transcript a. Consequently, two approaches were applied to detect the expression of transcript a in MCF-7 cells; 1) To allow for the detection of the intronless transcript a, the RNA samples were treated with DNase I and each was analysed after reverse transcription in the presence of reverse transcriptase (+RT) or mock reverse transcription in the absence of reverse transcriptase (−RT); 2) each RNA sample was amplified by two pairs of specific primers SHONF1/SHONc3 and SHONc5/SHONc3 (Table 1). The SHONc5 forward primer was specific for transcripts a and b, SHONF1 forward primer for transcripts b and c, and SHONc3 reverse primer for all the three transcripts (a, b and c).

TABLE 1
Primer sequences used to detect gene specific transcripts by
RT-PCR (SEQ ID NOS 18, 14, 19, 14, 19, 14, 20 and 21,
respectively, in order of appearance).

| | | | Amplicon |
|---|---|---|---|
| Genes | Primer | Name: Sequence (5′-3′) | (bp) |
| SHON a/b | Forward | SHONc5: ATGCCCATCAAAAGACTCTCACT | 280 |
| | Reverse | SHONc3: ACTTCCCTAAAGCTTGAAAGTGG | |
| SHON b | Forward | SHONF1: ATGGATCGGATGGCCAGCTCCA | 456 |
| | Reverse | SHONc3: ACTTCCCTAAAGCTTGAAAGTGG | |
| SHON c | Forward | SHONF1: ATGGATCGGATGGCCAGCTCCA | 338 |
| | Reverse: | SHONc3: ACTTCCCTAAAGCTTGAAAGTGG | |
| β-ACTIN | Forward | ACTIN5: TTCCTGGGCATGGAGTC | 84 |
| | Reverse: | ACTIN3: CAGGTCTTTGCGGATGTC | |

mRNA Expression Assays

For mRNA expression studies, a cDNA panel (Primgen) consisting of 10 ng of first-strand cDNA from various human tissues was screened by PCR with the SHON specific primers. As recommended by the manufacturer, human β-2-microglobulin gene was used as the cDNA input control. The expression of SHON in a variety of cancer cell lines was also determined by RT-PCR using SHON a/b specific primers SHONc5/SHONc3 (Table 1). The cell lines included MCF-7, T47D, BT474, BT459 and MDA-MB-231 human breast cancer cells; DU145, PC3 and LnCap human prostate cancer cells; RL95-2 and AN3 endometrial carcinoma cells; Ovca4 ovarian cancer; A549 and H1975 human lung carcinoma cells; AGS and MKN-45 human stomach cancer cells.

Immunohistochemical Analysis of SHON Protein in Mammary Carcinoma

Study patients—This was a retrospective study in a consecutive series of 1237 patients with early stage primary invasive breast cancers who were diagnosed from 1990 to 1999 and entered into the Nottingham Tenovus Primary breast cancer series. This is a well-characterized series of patients under the age of 71 years (median, 55 years) with long-term follow-up. All patients were treated uniformly in a single institution. Detailed patient demographics and clinicopathological characteristics were routinely assessed (Table 2) (Pinder et al., 1994). Patients received standard surgery (mastectomy or wide local excision) with radiotherapy. Based on well characterised prognostic and predictive factor status including Nottingham Prognostic Index (NPI), estrogen receptor-α (ER) status, and menopausal status, adjuvant systemic therapy (AT) was scheduled. Patients with NPI scores of <3.4 (low risk) did not receive AT. Patients with ER positive tumours and NPI scores of >3.4 (high risk) were given endocrine therapy (ET). In pre-menopausal patients with NPI scores of ≥3.4 (high risk) classical Cyclophosphamide, Methotrexate, and 5-Flourouracil (CMF) chemotherapy was given; and patients with ER positive tumour were also offered ET. Postmenopausal patients with NPI scores of >3.4 and ER positive were offered ET, while ER negative patients received classical CMF chemotherapy (Elston and Ellis, 1991).

TABLE 2
Clinicopathological characteristics of the whole cohort.

| Variable | n* | Cases | (%) |
|---|---|---|---|
| Menopausal status | 1650 | | |
| Pre-menopausal | | 612 | (37.0) |
| postmenopausal | | 1038 | (63.0) |
| Tumour Grade (NGS) | 1650 | | |
| G1 | | 306 | (18.5) |
| G2 | | 531 | (32.2) |
| G3 | | 813 | (49.3) |
| Lymph node stage | 1650 | | |
| Negative | | 1056 | (64.0) |
| Positive (1-3 nodes) | | 486 | (29.5) |
| Positive (>3 nodes) | | 108 | (6.5) |
| Tumour size (cm) | 1650 | | |
| T1 a + b (≤1.0) | | 187 | (11.0) |
| T1 c (>1.0-2.0) | | 868 | (53.0) |
| T2 (>2.0-5) | | 5729 | (35.0) |
| T3 (>5) | | 16 | (1.0) |
| Tumour type | 1650 | | |
| IDC-NST | | 941 | (57) |
| Tubular | | 349 | (21) |
| ILC | | 160 | (10) |
| Medullary (typical/atypical) | | 41 | (2.5) |
| Others | | 159 | (9.5) |
| NPI subgroups | 1650 | | |
| Excellent PG (2.08-2.40) | Low risk | 207 | (12.5) |
| Good PG (2.42-3.40) | | 331 | (20.1) |
| Moderate I PG (3.42 to 4.4) | High risk | 488 | (29.6) |
| Moderate II PG (4.42 to 5.4) | | 395 | (23.9) |
| Poor PG (5.42 to 6.4) | | 170 | (10.3) |
| Very poor PG (6.5-6.8) | | 59 | (3.6) |
| Survival at 20 years | 1650 | | |
| Alive and well | | 1055 | (64.0) |
| Dead from disease | | 468 | (28.4) |
| Dead from other causes | | 127 | (7.6) |
| Adjuvant systemic therapy (AT) | 1602 | | |
| No AT | | 665 | (42.0) |
| Hormone therapy (ET) | | 642 | (41.0) |
| Chemotherapy | | 307 | (20.0) |
| Hormone + chemotherapy | | 46 | (3.0) |
| SHON expression | 1237 | | |
| Negative | | 471 | (38%) |
| Positive | | 766 | (62%) |

*Number of cases for which data were available.
NPI, Nottingham Prognostic Index;
PG, prognostic group

TABLE 3
Antigens, primary antibodies, clone, source, optimal dilution and scoring system
used for each imrnunohistochemical marker.

| | | | | | | Scoring | | |
|---|---|---|---|---|---|---|---|---|
| Antigen^{a} | Antibody | Clone | Source | Dilution | Distribution | system | Cut-offs | Refs. |
| p53 | Mouse | DO7 | Novocastra | 1:50 | Nuclear | % | >10% | b |
| | MAb anti | | | | | positive | (negative) | |
| | p53 | | | | | cells | 10-20 | |
| | | | | | | | (low) | |
| | | | | | | | >20% | |
| | | | | | | | (High) | |
| Bcl2 | Mouse | 124 | Dako- | 1:100 | Cytoplasm | % | >10% | b |
| | MAb anti- | | Cytomation | | | positive | (positive | |
| | Bcl2 | | | | | cells | | |
| BRCA1 | BRCA1 | MS110 | Oncogen | 1:150 | Nuclear | % | <25% | b |
| | | | Research | | | positive | (negative) | |
| | | | | | | cells | | |
| ATM | Rabbit | Y170 | Abcam | 1:100 | Nuclear | % | <25% | b |
| | MAb anti- | | | | | positive | (negative) | |
| | ATM | | | | | cells | ≥75% | |
| | | | | | | | (positive) | |
| p27 | anti-p27 | SX53G8 | Dako- | 1:50 | Nuclear | % | ≥10% | b |
| | | | Cytomation | | | positive | (positive) | |
| | | | | | | cells | | |
| Vimentin | Mouse | Vim 3B4 | Dako- | 1:250 | Cytoplasm | % | ≥10% | b |
| | MAb anti- | | Cytomation | | | positive | (positive) | |
| | vimentin | | | | | cells | | |
| Bax | Rabbit | Polyclonal | Abcam | 1:1000 | Cytoplasm | % | ≥10% | b |
| | anti-Bax | | | | | positive | (positive) | |
| | | | | | | cells | | |
| ER | Mouse | 1D5 | Dako- | 1:200 | Nuclear | Allred | ≥3 | b |
| | MAb anti- | | Cytomation | | | score | (positive) | |
| | ER-α | | | | | | | |
| PR | Mouse | PR | Dako- | 1:150 | Nuclear | Allred | >3 | b |
| | MAb anti- | | Cytomation | | | score | (positive) | |
| | PR | | | | | | | |
| EGFR | Mouse | 31G7 | Zymed | 1:50 | Membrane | 0-3 as | 0 or +1 | b |
| | MAb anti- | | | | | HER2 | (negative) | |
| | EGFR | | | | | | +2 or +3 | |
| | | | | | | | (positive) | |
| CK14 | Mouse | LL002 | Novocastra | 1:40 | Cytoplasm | % | ≥10% | b |
| | MAb anti- | | | | | positive | (positive) | |
| | Ck14 | | | | | cells | | |
| Ck5/6 | Mouse | D5/161B4 | Chemicon | 1:60 | Cytoplasm | % | ≥10% | b |
| | MAb anti- | | | | | positive | (positive) | |
| | Ck5/6 | | | | | cells | | |
| Ck17 | Mouse | E3 | Dako- | 1:100 | Cytoplasm | % | ≥10% | b |
| | MAb anti- | | Cytomation | | | positive | (positive) | |
| | Ck17 | | | | | cells | | |
| Ck18 | Mouse | DC10 | Dako- | 1:100 | Cytoplasm | % | ≥10% | b |
| | MAb anti- | | Cytomation | | | positive | (positive) | |
| | Ck18 | | | | | cells | | |
| HER2 | Rabbit | polyclonal | Dako- | 1:100 | Membrane | Asco | | c |
| | antihuman | | Cytomation | | | guideline | | |
| | c-erbB2 | | | | | | | |
| Ki67 | Mouse | MIB1 | Dako- | 1:300 | Nuclear | % | ≤30% | b |
| | MAb anti- | | Cytomation | | | positive | (low) | |
| | Ki-67 | | | | | cells | >30% | |
| | | | | | | | (high) | |
| TOP2A | Mouse | KiS1 | Dako- | 1:150 | Nuclear/ | % | >25% | b |
| | MAb | | Cytomation | | cytoplasm | positive | (positive) | |
| | TOP2A | | | | | cells | | |
| p21 | Mouse | EA10 | Abcam | 1:25 | Nuclear | % | ≥10% | b |
| | MAb anti- | | | | | positive | (positive) | |
| | p21 | | | | | cells | | |

^{a}All sections were pre-treated with microwave antigen retrieval using 0.1% citrate buffer (pH6) except for HER2 (no pre-treatment) and EGFR (pre-treated with protease for 10 minutes).
b references (Callagy et al., 2006; Tan et al., 2008; Abdel-Fatah et al., 2010a; Abdel-Fatah et al., 2010b).
c references (Sauter et al., 2009).
MDM2, murine double minute 2;
MDM4, murine double minute 4;
ATM, ataxia telangiectasia mutated;
BRCA1, breast cancer 1 early onset;
ER, estrogen receptor;
PR, progesterone receptor;
CK, cytokeratin;
EGFR, epidermal growth factor;
TOP2A, topoisomerase II alpha;
MAb, monoclonal antibody.

Exploratory subgroup analysis of SHON expression was also performed in lymph node (LN) negative vs. LN positive cases, high risk patients (NPI>3.4) who received AT vs. AT-naïve cases and in ER+ high risk patients who received ET vs. ET-naïve cases.

Survival data—Survival data including survival time, disease-free survival (DFS), and development of loco-regional and distant metastases (DM) were maintained on a prospective basis. Median follow up was 111 months (range 1 to 233). DFS was defined as the number of months from diagnosis to the occurrence of local recurrence, local LN relapse or DM relapse. BC specific survival (BCSS) was defined as the number of months from diagnosis to the occurrence of BC related-death. DM-free survival was defined as the number of months from diagnosis to the occurrence of DM relapse. Survival was censored if the patient was still alive, lost to follow-up, or died from other causes.

The Reporting Recommendations for Tumour Marker Prognostic Studies (REMARK) criteria (McShane et al., 2005) were followed throughout this study. This work was approved by the Nottingham Research Ethics Committee.

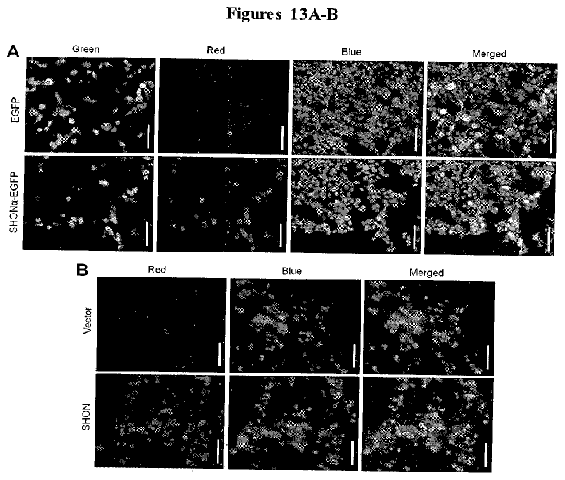

Tissue microarrays (TMAs) and immunohistochemistry (IHC)—Tumours were arrayed in tissue microarrays (TMAs) constructed with 2 replicate 0.6 mm cores from the centre and periphery of the tumours for each marker. The TMAs were immunohistochemically profiled for SHON and other biological antibodies (Table 3) as previously described (Abdel-Fatah et al., 2010a; Abdel-Fatah et al., 2010b). Immunohistochemical staining was performed using NOVOLINK Detection kit according to manufacturer's instructions (Leica Microsystems). The specificity of the rabbit polyclonal antibody against SHONα in the immunohistochemical analysis of paraffin-embedded specimens was validated (FIG. 13). TMAs sections were incubated at room temperature for one hour with the SHONα antibody at 1:700 dilution. Pre-treatment of TMAs section was performed with citrate/EDTA buffer (pH 6.0) for 20 minutes. To validate the use of TMAs for immuno-phenotyping, full-face sections of 40 cases were stained and the protein expression levels of the different antibodies were compared. Positive and negative (omission of the primary antibody and IgG-matched serum) controls were included in each run.

Evaluation of immunohistochemical staining—The invasive tumour cells within TMA cores were evaluated blinded to the clinico-pathological characteristics of patients in two different settings. There was excellent intra and inter-observer agreements (k>0.8; Cohen's κ and multi-rater κ tests, respectively). Whole field inspection of the core was scored and intensities of nuclear staining were grouped as follows: 0=no staining, 1=weak staining, 2=moderate staining, 3=strong staining. The percentage of each category was estimated. Nuclear staining was recorded as a continuous variable using percentages. In addition, H-score was calculated as previously described (Abdel-Fatah etal., 2010a; Abdel-Fatah etal., 2010b). Positive SHON (SHON+) expression was defined by median as the presence of nuclear staining in >10% of malignant cells. Not all cores within the TMA were suitable for IHC analyses due to the fact that some cores were missing or lacked sufficient tumour cells.

Statistical analysis—Data analysis was performed using SPSS (SPSS, version 17 Chicago, Ill.). Where appropriate, Pearson's Chi-square, Fisher's exact, Student's t and ANOVAs one way tests were used. Cumulative survival probabilities were estimated using the Kaplan-Meier method and differences between survival rates were tested for significance using the log-rank test. Multivariate analysis for survival was performed using the Cox hazard model. The proportional hazards assumption was tested using standard log-log plots. Hazard ratios (HR) and 95% confidence intervals (95% CI) were estimated for each variable. All tests were two-sided with a 95% CI and a p value <0.05 was considered significant. For multiple comparisons, p values were adjusted according to Holm-Bonferroni correction method (Holm, 1979).

### Example 2

### Results

Observations and Discussions

Identification of SHON

PIKR2786 (Genentech UNQ ID: UNQ2786) represented by an EST (GenBank accession number AY358103) was originally identified, through bioinformatic analysis, as a potential secreted and trans-membrane protein from a large-scale effort to identify human secreted and trans-membrane proteins (Clark et al., 2003). However, its biological function is undetermined. Sequence homology searches using NCBI BLAST revealed that UNQ2786 belongs to a hominoid-specific gene family with no known orthologs outside the primate lineage. We have demonstrated that UNQ2786 is a secreted protein and is a human mammary epithelial oncogene and have thus renamed it as SHONα.

To identify the full length SHONα mRNA we carried out 5′RACE (rapid amplification of 5′ complementary DNA ends) analysis, to extend the 5′ end sequence of the EST AY358103, on mRNA isolated from the mammary carcinoma cell line, MCF-7. We did not identify additional upstream sequence for the EST by 5′RACE. Because two more transcripts with identical 3′ ends to the EST were identified, the original EST was designated SHON transcript a, and accordingly the two newly-identified ones as SHON transcripts b and c.

SHON transcript a and protein isoform α—The complete cDNA of human SHON transcript a was 725 nucleotides long (FIG. 1). Two transcription initiation sites were identified in the 5′RACE at nucleotide position 1 (T) and 21 (C), respectively. The presence of initiation site 1 was confirmed in 7 clones and site 2 in 4 clones. Both contained an open reading frame of 282 bp. The conserved standard AATAAA polyadenylation signal was also found in the 3′UTR. The protein encoded by SHONa was termed SHONα, which contained 93 amino acid residues with a molecular mass of 9.7 kDa and an isoelectric point of 7.8 (FIG. 2). SHONα protein was predicted to be a soluble protein with an average hydrophobicity of 0.287096. A signal leading peptide MPIKRLSLLCLPSSVLASIPS (residues 1-21) (SEQ ID NO: 22) was also predicted by SignalP 3.0 (Bendtsen et al., 2004). This analysis also suggested that the matured SHONα protein may form an internal disulfide bond between the two cysteines (at nucleotide positions 25 and 43), contain a potential protein kinase C phosphorylation site (TAR 54-56), an N-glycosylation site (NQTL 73-76 (SEQ ID NO: 23)), a cAMP and cGMP-dependent protein kinase phosphorylation site (KRLS 4-7 (SEQ ID NO: 24)) and an Nmyristoylation site (GVFPTQ 77-82 (SEQ ID NO: 25)).

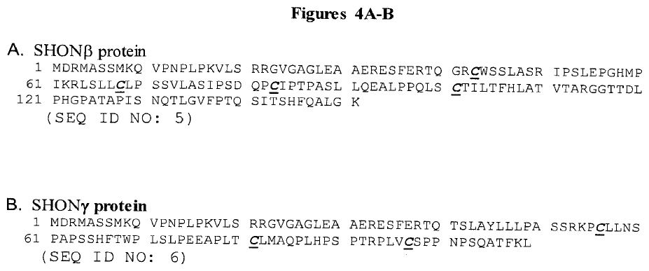

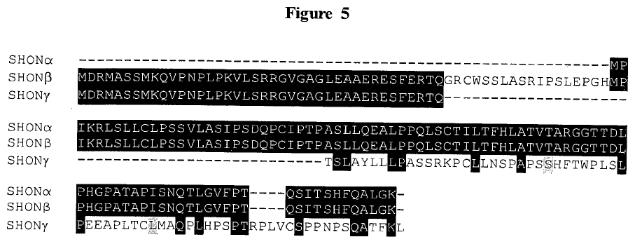

SHON transcript b and protein isoform β—Only one transcription initiation site for SHON transcript b was identified in the 5′RACE in MCF-7 cells at nucleotide position 21 (T) (FIG. 3A). The initiation site was confirmed by 9 clones. The 5′ end upstream sequence was extended based on an EST clone (IMAGE: 1286243) from human tonsillar cells (GenBank accession numbers CR745472 and AA740612). Thus the complete human SHONb cDNA was 893 nucleotides long (FIG. 3A). There were three potential in frame translation ATG initiation codons. The most upstream one predicted an open reading frame of 456 bp which encoded a peptide (SHONβ) of 151 amino acid residues with a molecular mass of 16.0 kDa and an isoelectric point of 9.2 (FIG. 4A). Since the translation start codon ATG of transcript a is in frame with transcript b, both SHON α and β proteins have identical amino acid sequences at the C-termini (FIG. 5). Like the SHONα isoform, SHONβ was also predicted to be a soluble protein with an average hydrophobicity of −0.060927. However, it was not predicted to be a secreted protein. SHONβ protein may form two internal disulfide bonds between the four cystines ((43,68) and (83,101)) and contain one potential cAMP- and cGMP-dependent protein kinase phosphorylation site (KRLS (62-65) (SEQ ID NO: 24)) and three potential protein kinase C phosphorylation sites (SMK(7-9), SRR(20,22) and TAR(112-114), an N-glycosylation site (NQTL(131-134) (SEQ ID NO: 23)) and an N-myristolylation site (GVGAGL(23-28) (SEQ ID NO: 26), GVFPTQ(135-140) (SEQ ID NO: 25)).

SHON transcript c and protein isoform γ—A third transcript variant was identified in the expression test of SHON in MCF-7 cells by RT-PCR (see below), and termed SHONc. Compared with transcription variant SHONb, transcript c had a proximal deletion of 118 bp in exon 2, which resulted from alternative splicing by use of a downstream acceptor (FIG. 3). The complete human SHONc cDNA was 775 nucleotides long (FIG. 3B). There were three potential in frame ATG initiation codons. The most upstream ATG codon predicted an open reading frame of 330 bp, which encoded a peptide (SHONγ) of 109 amino acid residues with a molecular mass of 11.8 kDa and an isoelectric point of 9.4 (FIG. 4B). The SHONγ isoform was also predicted to be a soluble protein with an average hydrophobicity of −0.269725. SHONγ protein may form one internal disulfide bond between two (nucleotide positions 56 and 97) of the three cysteines and contain one potential Casein kinase II phosphorylation site (SLPE(72-75) (SEQ ID NO: 27)) and four potential protein kinase C phosphorylation sites (SMK(7-9), SRR(20-22), SSR(51-53), TFK(106-108) and an N-myristolation site GVGAGL(23-28) (SEQ ID NO: 26)). The deletion in exon 2 in the SHONc transcript caused a reading frame shift. Therefore, SHON isoform γ had an identical N terminal amino acid sequence to isoform β but a distinctive C terminal sequence compared with isoform β (FIG. 5).

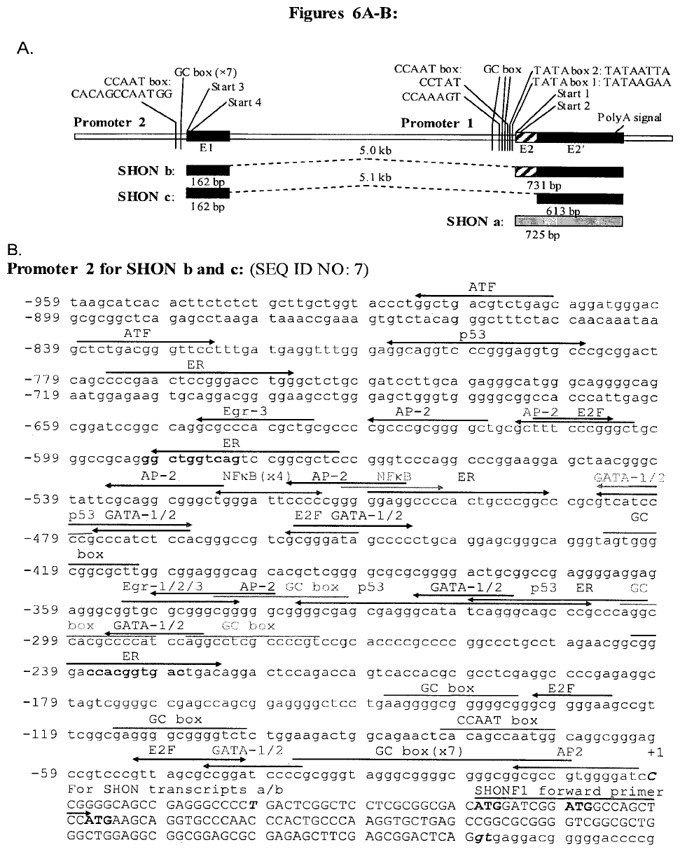

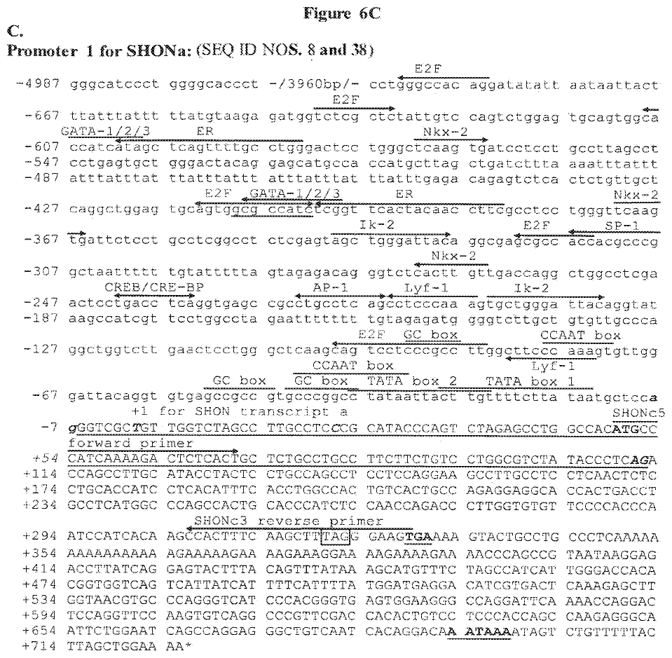

Genomic structure of SHON genes—Sequence comparison of SHON transcripts with the genomic sequence (GenBank accession number NT_007933) revealed that transcript a contains a single exon, whereas b and c contain two exons (FIG. 6). Compared with transcript b, transcript c results from the use of a downstream acceptor for mRNA splicing, generating the 118 bp deletion in the second exon. A single intron was located between nucleotide positions 162 and 163 in the pre-mRNA of SHON transcripts b and c, and was 5,000 and 5,118 bp long, respectively. All of the exon/intron splice junctions followed the GT-AG rule (FIG. 6B). In both SHON b and c genes, exon 1 encoded the 5′ UTR of the gene and the N-terminal amino acid residues while exon 2 encoded the 3′ UTR and the C-terminal residues.

The three transcripts were transcribed from two different promoters (FIG. 6A-C). Transcript a was regulated by one promoter whereas transcripts b and c by the second. The two promoters were approximately 5 kb apart.

Promoter 1 for SHONa: SHONa is a single exon gene with a promoter situated within the large intron of SHON b and c gene (FIG. 6A-B). Two putative overlapped TATA boxes, TTATAAGAAAACAAG (SEQ ID NO: 28) (or in reverse CTTGTTTTCTTATAA (SEQ ID NO: 29)) and CTATAATTACTTGT (SEQ ID NO: 30), were located at −15 and −25 respectively in the SHONa promoter. The core of the latter, TATAATTA matches the functional TATA box reported in PISRT1 gene (Pailhoux et al., 2001). Two putative CCAAT boxes (−35 and −73) were also identified. The one at −73 (CCAAAGT) is similar to the Inr consensus sequence of PyPyANA/TPyPy that surrounds the transcription initiation site (Smale and Baltimore, 1989) and the other (CCTAT) at −35 is found to bind NF-Y in the promoter region of human MHCII Dpa (Turco et al., 1990) and cdc25 genes (Zwicker et al., 1995). In addition, in the proximal region of SHONa promoter were three GC boxes at -38, -47 and -88, respectively. A search for additional promoter elements by TFSEARCH (Parallel Application TRC Laboratory, Real World Computing Project, Japan) revealed SHONa gene promoter sequence (nucleotides −697 to −1) identified potential transcription factor binding sites, among others, for SP1, AP-1, E2F, Lyf-1, Ik-2, CREB, CRE-BP, Nkx-2 and TATA-1/2/3 (FIG. 6B). Therefore, the transcription start sites identified in the 5′RACE analysis most likely represented the actual transcription start sites of the full length of SHONa transcript.

The SHONa gene spanned approximately 1 kb of genomic DNA. The translation start codon ATG was in frame with SHONb; therefore, SHONα protein represented an N-terminal “truncated” version of SHONβ with identical sequence to the C-terminal of SHONβ (FIG. 5).

Promoter 2 for SHON b and c: Sequence analysis showed that the SHON b and c gene promoter sequence (nucleotides −959 to −1) was GC rich (FIG. 6C). It contained several putative GC boxes e.g. at nucleotides −18, −101 and −135; a CCAAT box at −70. A search for additional promoter elements by TFSEARCH revealed potential transcription factor binding sites, among others, for SP1, AP-2, E2F, p53, TATA-1/2, Egr-1/2/3, NFKB and ATF (FIG. 6C).

SHONα is Highly Expressed in the MCF-7 Breast Cancer Cell Line

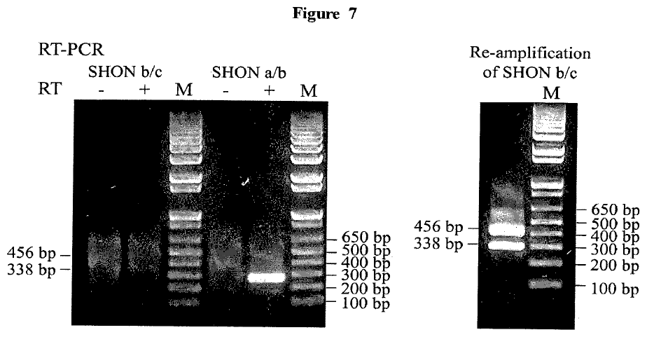

RT-PCR was used to detect the relative expression of SHON transcript variants in MCF-7 cells. Since transcript a is intronless it was not possible to design primer pairs only specific to transcript a. Therefore, two pairs of primers SHONc5/SHONc3 and SHONF1/SHONc3 (Table 1) were used to amplify transcripts for a/b (of 280 bp for both) and b/c (456 bp for b and 338 bp for c), respectively. As shown in FIG. 7 (left panel), weak bands (456 bp for transcript b and 338 bp for c) were observed in the RT-PCR using the SHONF1/SHONc3 primers, demonstrating that the existence of low level expression of SHON transcripts b and c in MCF-7 cells. After re-amplification the two bands were clearly visible (FIG. 7, right panel) and their sequence was verified by DNA sequencing. Under the same conditions, a band of 280 bp for both transcripts a and b was observed in the RT-PCR using the SHONc5/SHONc3 primers. The much stronger signal indicated that transcript a was relatively more abundant since the amount of transcript b was very low. Given that SHONc5/SHONc3 primers amplified a specific band only in the presence of reverse transcriptase, transcript a was therefore confirmed as being intronless. Thus MCF-7 cells expressed all three transcripts and transcript a was the most abundant transcript of all three variants.

Anti-SHONα Polyclonal Antibody Production and Purification

SHONα was predicted as a secreted protein and the cDNA of mature SHONα was cloned into the pGEX-4T1 vector (Amersham Biosciences, Piscataway, N.J., USA) to generate pGEX-4T1-SHONα for the expression of glutathione-S-transferase (GST) recombinant protein GST-SHONα in bacteria. Purified recombinant GST-SHONα protein was used as antigen to immunilise rabbits to generate rabbit polyclonal anti-SHONα antibodies. SHONα antibodies were then affinity-purified from rabbit antisera sequentially. Anti-GST antibodies in the antisera were removed by passing through columns of GST protein covalently linked to Glutathione Sepharose 4B beads. SHONα-specific antibodies were then purified by using GST-SHONα protein covalently bound to Glutathione Sepharose 4B beads.

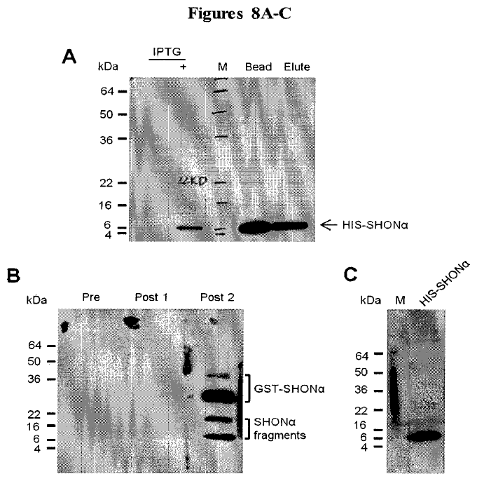

The affinity-purified rabbit polyclonal anti-SHONα antibody was able to specifically recognize the recombinant GST-SHONα protein and a HIS tagged SHONα protein (HIS-SHONα) by Western blotting (FIG. 8A-8C).

Specificity of the Rabbit Anti-SHONα Polyclonal Antibody

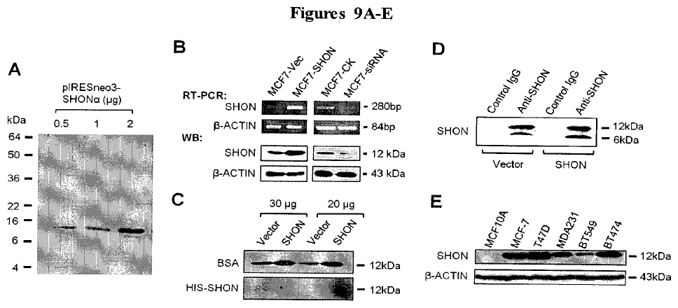

MCF-7 cells were transiently transfected with the SHONα expression plasmid pIRESneo3-SHONα, and transfected cells were then lysed for Western blot analysis. As shown in FIG. 9A, the rabbit polyclonal anti-SHONα antibody was able to detect the forced expression of SHONα protein from the plasmid as a specific band of 12 kDa. Enhanced signals were observed as the amounts of the plasmid used for the transfection increased.

To test whether SHONα antibody can recognise endogenous SHON protein, MCF-7 cells were stably transfected with the SHONα expression plasmid pIRESneo3-SHONα (designated MCF7-SHON) and the empty control pIRESneo3 vector (MCF7-Vec), or with the SHON siRNA plasmid pSilencer-siRNA (MCF7-siRNA) or the negative siRNA control plasmid pSilencer-CK (MCF7-CK). Forced expression of SHONα in MCF7-SHON cells was verified at the mRNA by RT-PCR (FIG. 9B, top left panel). When the whole cell lysates were subjected to Western blot analysis, the affinity purified SHONα antibody was able to detect both the endogenous and forced expression of SHON protein (FIG. 9B, bottom left panel). This was further confirmed by siRNA mediated knock-down of the endogenous SHON in MCF-7 cells. The depletion of SHON by SHON siRNA in stable MCF7-siRNA cells, compared with the siRNA control MCF7-CK cells, was verified by RT-PCR (FIG. 9B, top right panel). The SHONα antibody was able to detect the reduced expression of SHON in MCF7-siRNA cells (FIG. 9B, bottom right panel).

To verify the specificity of the band detected by the SHONα antibody on the Western blots, antigen peptide blocking experiment was carried out. As shown in FIG. 9C, preincubation of the SHONα antibody with the recombinant HIS-SHONα peptide completely blocked the activity of the antibody to detect the endogenous and forced expression of SHON protein in MCF-7 cells.

To test whether SHONα antibody can recognise native protein, immunoprecipitation assays were performed. The whole cell lysates of stable MCF7-Vec and MCF7-SHON cells were subjected to immunoprecipitation with the affinity purified SHONα antibody. As shown in FIG. 9D, both the endogenous and forced expression of SHON proteins were effectively pulled-down by the SHONα antibody as a full-length of 12 kDa band whereas the normal control rabbit IgG did not.

To further validate the SHONα antibody, Western blot analysis was carried out with several other human breast cancer cell lines and the normal but immortalised human mammary epithelial cell line MCF10A. As shown in FIG. 9E, apart from MCF-10A cell line, other breast cancer cell lines MCF-7, T47D, MDA231, BT549 and BT474, all expressed SHON at various levels as detected by the SHONa antibody. However, MCF10A did not express SHON.

SHON is Highly Expressed in Cancer Cell Lines and Cancer Tissues

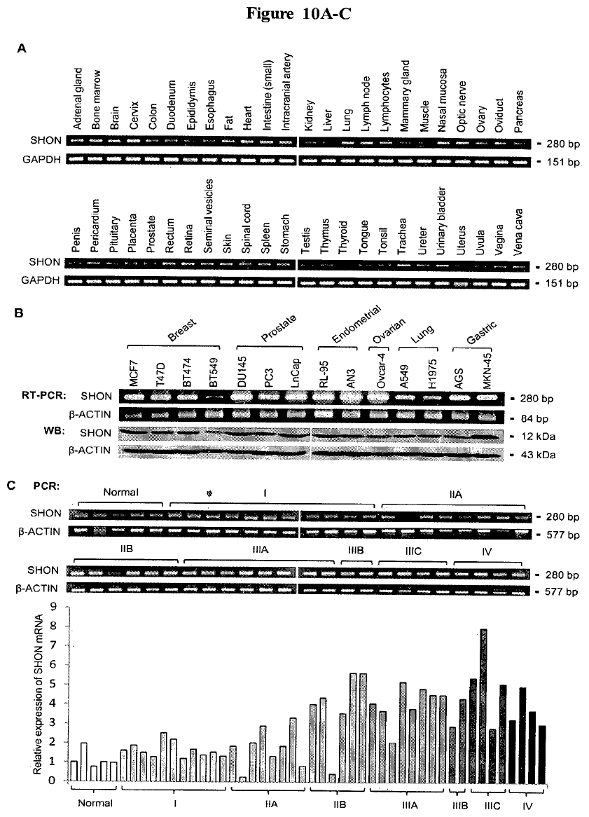

We determined the expression of SHON mRNA in normal human tissues by PCR using a commercially available panel of cDNAs (FIG. 10A). SHON gene expression was detected in all 48 tissues tested, but at a low level since 40 cycles of amplification was needed. Relative higher expression was observed in adrenal gland, bone marrow, brain, cervix, rectum, retina, trachea and urinary bladder, whereas other remaining tissues including mammary gland exhibited lower expression.

We next determined the expression of SHON in a number of human cancer cell lines by RT-PCR. SHON mRNA was expressed in all the cancer cell lines tested, including breast, prostate, endometrial, ovarian, lung and gastric cancer cell lines (FIG. 10B, top panel). The expression of SHON protein in these cell lines was examined by western blot using the affinity purified rabbit polyclonal SHONα antibody. Consistent with the RT-PCR results, the rabbit polyclonal antibody detected a specific band of the expected size (12 kDa) in all the cancer cell lines tested (FIG. 10B, lower panel). However, no SHON protein expression was detected in the normal human mammary epithelial cell line, MCF10A (FIG. 9E).

To determine the clinical relevance of SHON expression in human mammary carcinoma, we assessed SHON mRNA expression in a commercially available a Breast Cancer cDNA Array (OriGene) by PCR. As shown in FIG. 10C, SHON mRNA was expressed in both normal and breast cancer tissues. However, an increased SHON mRNA expression was observed in cancer tissues compared with the normal breast tissues. Moreover, the expression level of SHON mRNA was positively correlated with cancer stages (FIG. 10C, bottom panel).

SHONα is a Secreted Protein

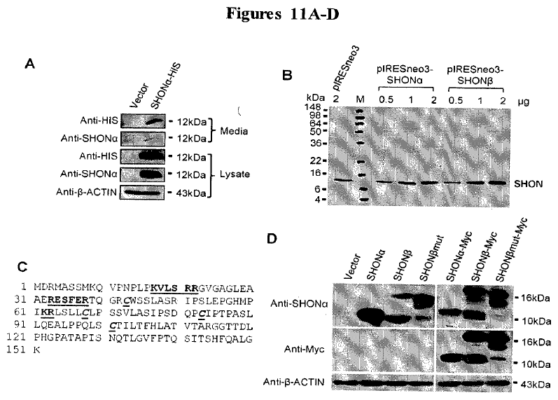

Since SHONα was the major isoform expressed in MCF-7 cells and contained a putative signal peptide for protein secretion at its N-terminal, the open reading frame of SHONα was cloned into the mammalian expression vector pIRESneo3 with a HIS epitope tag at the C-terminal (designated as pIRESneo3-SHONα-HIS). This plasmid and the empty vector were transiently transfected into HEK293 cells, which are readily transfected. As shown in FIG. 11A, HIS-tagged SHONα protein can be readily detected in both the whole cell lysates and the conditioned media by Western blotting using either an anti-HIS monoclonal antibody or the anti-SHOINα polyclonal antibody.

SHONβ is a Proprotein

Both SHON α and β have identical c-terminal sequence but differ in their N-terminal sequence as the β isoform has an extended N-terminal sequence of 58 residues and does not have a putative signal peptide. Interestingly, when the SHONα and β expression plasmids (pIRESneo3-SHONα and pIRESneo3-SHONβ) were transiently transfected into MCF-7 cells, the larger SHONβ isoform was not detected by Western blotting with the rabbit polyclonal antibody to the C-terminal of SHONα, which is identical to that of SHONβ. Instead, a band of approximately the same size as that of endogenous SHON in MCF-7 cells was detected for both isoforms (FIG. 11B), indicating that SHONβ may be a proprotein.

Proprotein convertases usually catalyse the release of protein hormones and neuropeptides from their precursors, for example pro-opiomelanocortin, prorenin, proenkephalin, prodynorphin, prosomatostatin and proinsulin. Precursors are usually cleaved at the consensus motif (K/R)X n(K/R)↓, where n=0, 2, 4, or 6 and X is usually not a Cys (Karim-Jimenez et al., 2000; Khatib et al., 2001). SHONβ contains three such conserved motifs: KVLSRR (SEQ ID NO: 31), RESFER (SEQ ID NO: 32) and KR (FIG. 11C). To examine whether SHON is a substrate of protein convertases, a SHONβ mutant expression plasmid (pIRESneo3-SHONβ was generated, in which the third conserved motif K⁶²R⁶³ was mutated into N⁶²I⁶³. A second vector containing the N⁶²I⁶³ mutation and a c-Myc tag at the C-terminal was also generated (designated pIRESneo3-SHONβni-Myc). These plasmids were transiently transfected into HEK293 cells. Western blot analysis with the rabbit anti-SHONα antibody detected a protein band of approximately 10 kDa, similar to the molecular weight of SHONα, in HEK293 cells transiently transfected with pIRESneo3-SHONβ (FIG. 11D, top left panel). In addition, a larger band of approximately 16 kDa was observed which may represent the unprocessed SHONβ proprotein. Expression of this potential proprotein was not observed in MCF-7 cells (FIG. 11B). However, transient transfection of the mutated vector, pIRESneo3-SHONβni significantly reduced the intensity of the 10 kDa band, while a stronger 16 kDa was detected in addition, a unique intermittent fragment of 14 kDa was observed. These results indicate that mutation of the K⁶²R⁶³ motif affected convertase cleavage of the proprotein. Similar results were obtained with c-Myc tagged SHONα and β expression plasmids (FIG. 11D, top right panel). Western blotting with the mouse monoclonal antibody 9E10 further confirmed the involvement of residues K⁶²R⁶³ in the cleavage (FIG. 11D, middle panel). These data clearly demonstrate that the residues K⁶²R⁶³ play an essential role in the maturation of SHON by proprotein convertases.

SHON is an Estrogen Inducible Gene

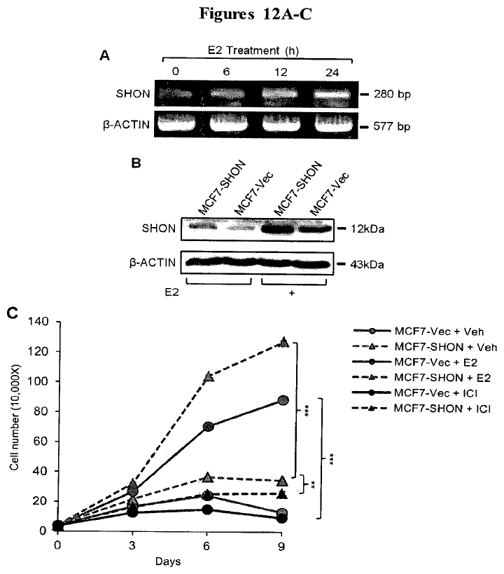

Within the breast cancer cell lines, SHON expression was higher in the three ER+ breast cancer cell lines MCF-7, T47D and BT474 compared with the ER− breast cancer cell lines BT549 and MDA-MB-231 (FIG. 9E and 10), indicating that SHON may be an estrogen regulated gene. We thus treated the ER+ MCF-7 cells with 17β-estradiol (E2). E2 treatment resulted in an increase in SHON mRNA levels in a time dependent manner (FIG. 12A). SHON protein expression was also increased in both MCF7-Vec and MCF7-SHON cells in response to E2 treatment (FIG. 12B). Therefore, SHON is an estrogen inducible gene.

Upon E2 treatment a synergistic increase in cell number was observed in MCF7-SHON cells compared with MCF-Vec cells whereas the ER pure antagonist ICI 182780 partially attenuated SHON-stimulated growth advantages in ER+ MCF-7 cells, indicating that SHON signaling is at least in part mediated by ER (FIG. 12C).

Clinicopathological Significance of SHON Expression

We have demonstrated that SHON is a novel mammary carcinoma oncogene. Forced expression of SHON significantly increases cancer cell proliferation, survival and migration/invasion. In addition, SHON enhances the oncogenicity of mammary carcinoma cell lines and is sufficient to oncogenically transform the human mammary epithelial cell MCF10A. Moreover, depletion of endogenous SHON expression or functional inhibition of SHON reduces cancer cell oncogenicity.

To determine the clinical relevance of SHON expression in human mammary carcinoma, we tested the suitability of the rabbit polyclonal SHONa antibody for immunohistochemical analysis. HEK293 cells were transiently transfected with the expression plasmid pIRESneo3-SHONα-EGFP expressing EGFP-tagged SHONα (SHONα-EGFP), or the pEGFP-C1 empty vector. SHONα-EGFP or EGFP proteins were viewed as green fluorescence using UV-visible fluorescence microscopy (FIG. 13A, far left panel). The expression of SHON was also immune-stained with the affinity purified rabbit polyclonal SHONα antibody as the primary antibody and visualised with a Cy5 cyanine dye conjugated secondary anti and body (Red). Stronger SHON signals were observed in pIRESneo3-SHONα-EGFP transfected cells, but not in pEGFP-C1 transfected control cells (FIG. 13A, 2^{nd} panel from the left). Moreover, the co-localisation of green and red fluorescence staining in the pIRESneo3-SHONα-EGFP transfected cells demonstrated that the rabbit anti-SHONα antibody specifically recognised SHON (FIG. 13A, far right panel). We also tested whether rabbit anti-SHONα antibody could recognise SHON protein in the formalin-fixed paraffin-embedded cell blocks. HEK293 cells were transiently transfected with the SHONα expression plasmid pIRESneo3-SHONα and the empty control pIRESneo3 vector and were used to prepare formalin-fixed paraffin-embedded cell blocks. As shown in FIG. 13B, the anti-SHONα antibody was able to detect SHON protein in the formalin-fixed paraffin-embedded sections after antigen retrieval. These results clearly demonstrated that the anti-SHONα antibody was suitable for use in immunohistochemical analysis.

We thus performed a large scale immunohistochemical analysis of SHON protein in mammary carcinoma using tissue microarrays (TMAs) made from the established ethical human tissue bank at the Nottingham University Hospitals NHS Trust. The TMAs contain an unselected cohort of 1,650 breast cancer tissues and have been used to study the frequency and variance of over 120 biomarkers by immunohistochemistry (Rakha et al., 2004; Abd El-Rehim et al., 2005; Putti et al., 2005; Rakha et al., 2005a; Rakha et al., 2005b; Rakha et al., 2006a; Rakha et al., 2006b; Rakha et al., 2007; Rolland et al., 2007; Green et al., 2008; Elsheikh et al., 2008).

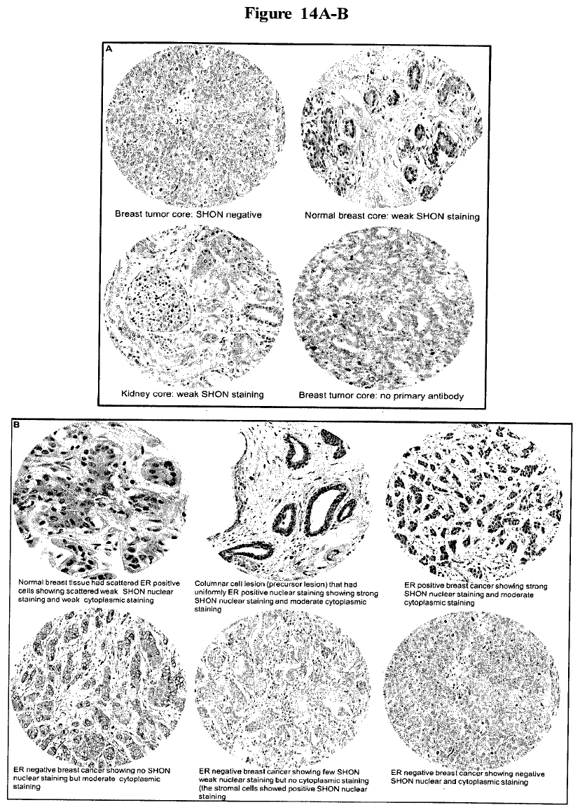

Normal breast terminal duct lobular units displayed strong nuclear and weak cytoplasmic expression of SHON throughout (FIG. 14A-B). A total of 1,237 tumours were suitable for analysis of SHON expression. 62% (766 out of 1237) of the tumours were SHON positive (Table 2). The associations of SHON expression in breast cancer with clinicopathologic features are presented in Table S4. Positive SHON expression was highly correlated to hormonal receptor status ER+, PR+ and AR+. In particular, SHON+ was significantly associated with ER+/PR+ breast tumours. There is a statistically significant correlation between SHON+ and HER2− or triple negative phenotype (P=0.004 and P<0.0001, respectively). SHON expression is very significantly related to many clinicopathological features including high grade, low proliferation index, high pleomorphism and tubular formation. SHON expression is also associated with tumor type and lymphovascular invasion. In addition, SHON− is strongly associated with epithelial mesenchymal transition phenotypes including low CK5/6+ and EGFR+, higher frequency of basal like phenotype and high level of vimentin. There is a weak but significant positive correlation between SHON and E-Cadherin (P=0.016). Furthermore, SHON expression is positively correlated to BRCA1, ATM and XRCC1 expression. SHON+ is negatively associated with p53 and p16 tumor suppressors, while positively associated with the expression of BCL-2 and TOP2A.

Survival Analyses

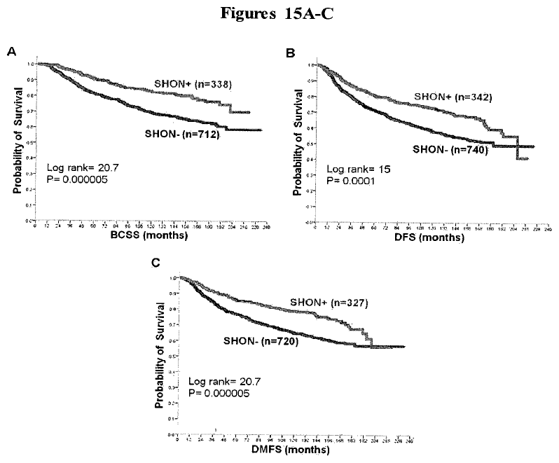

SHON− expression in breast tumours showed an adverse outcome at 10 years with a 2-fold increase in the risk of death (HR: 1.85, 95% CI: 1.4-2.4, p<0.00001), recurrence (HR: 5.5, 95% CI: 1.2-1.9, p=0.0001) and DM (HR: 1.6, 95% CI: 1.3-2.1, p=0.0001) compared to tumours with positive SHON expression (FIGS. 15A-C). Investigating the clinical outcome of 875 patients with early stage tumours confirmed that tumours with SHON− expression displayed a worse prognosis than those with SHON+ expression (data not shown).

Prognostic Significance of SHON Expression for Response to Endocrine Therapy in ER+ High Risk Breast Cancer Patients

In multivariate Cox regression model including other validated prognostic factors; lymph node stage, histological grade and tumour size (i.e., NPI components), SHON negative expression was confirmed as an independent predictor for clinical outcome in the study cohort breast cancer patients; HR: 1.4, 95% CI 1.1-1.9, p=0.017 (Table S5).

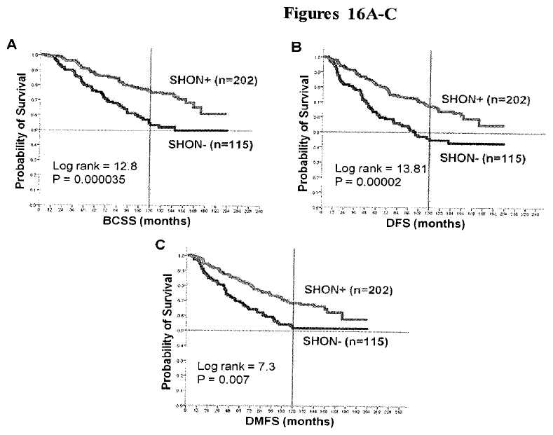

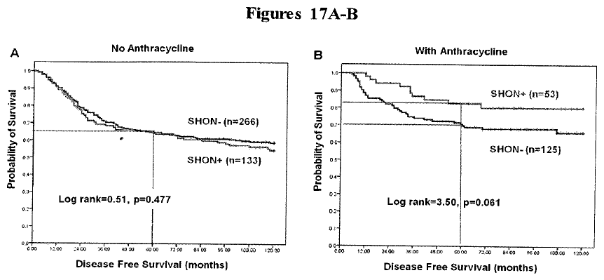

Moreover, SHON protein expression was found to be a prognostic marker for response to endocrine therapy for patients whose tumours were ER+and categorised as high risk (NPI≥3.4). Patients with tumours negative for SHON expression had a 2-fold increase in risk of death (HR: 2.1, 95% CI: 1.4-3.1, p<0.0001), recurrence (HR: 1.9, 95% CI: 1.4-2.6, p<0.0001) and distant metastasis (HR: 1.8, 95% CI: 1.2-2.5, p=0.007) at 10 years compared with patients whose tumours which were positive for SHON expression (FIG. 16A-C). However, SHON expression was not significantly related to disease free survival in the ER− patient cohort with or without anthracycline treatment (FIGS. 17A-B).

TABLE 4
Association between SHON expression and other clinicopathologic variables.

| SHON Expression | | |
|---|---|---|
| Negative | Positive | X² |

| | | | |
|---|---|---|---|
| Variables | n (%) | n (%) | Adjusted p-value |

Hormonal receptors

| | | | | |
|---|---|---|---|---|
| ER | (Negative) | 216 (32) | 50 (17) | 6.2 × 10 ⁻⁷*** |
| | (Positive) | 459 (68) | 251 (83) | |
| PR | (Negative) | 344 (50) | 93 (29) | 7.7 × 10 ⁻¹⁰*** |
| | (Positive) | 347 (50) | 226 (71) | |
| AR | (Negative) | 290 (44) | 64 (21) | 7.2 × 10 ⁻¹²*** |
| | (Positive) | 368 (56) | 239 (79) | |
| ER/PR | (ER−/PR−) | 195 (30) | 38 (13) | 1.7 × 10 ⁻⁸*** |
| | (ER−/PR+) | 17 (2) | 10 (4) | |
| | (ER+/PR−) | 129 (20) | 47 (16) | |
| | (ER+/PR+) | 311 (48) | 195 (67) | |

Pathological parameters

| | | | | |
|---|---|---|---|---|
| Grade^{#} | G1 | 97 (13) | 90 (27) | 6.9 × 10 ⁻¹⁶*** |
| | G2 | 206 (29) | 140 (42) | |
| | G3 | 426 (58) | 106 (31) | |
| Mitotic index | M1 (Low; mitoses < 10) | 216 (29) | 173 (53) | 1.2 × 10 ⁻¹⁵*** |
| | M2 (Medium; mitoses 10-18) | 122 (17) | 72 (21) | |
| | M3 (High; mitoses > 18) | 391 (54) | 91 (27) | |
| Pleomorphism | 1 (Small-regular uniform) | 14 (2) | 10 (3) | 2.7 × 10 ⁻¹⁶*** |
| | 2 (Moderate variation) | 230 (31) | 195 (58) | |
| | 3 (Marked variation) | 485 (67) | 131 (39) | |
| Tubular formation | (1) | 34 (5) | 20 (6) | 0.0006*** |
| | (2) | 234 (32) | 139 (41) | |
| | (3) | 61 (63) | 77 (53) | |
| Tumor type | Ductal | 471 (65) | 153 (46) | 2.2 × 10 ⁻¹⁰*** |
| | Medullary | 127 (17) | 85 (25) | |
| | Tubular | 21 (3) | 2 (1) | |
| | Lobular | 54 (7) | 58 (17) | |
| | Others | 55 (8) | 36 (11) | |
| Lymphovascular | (No) | 471 (67) | 245 (77) | 0.0013** |
| invasion | (Yes) | 230 (33) | 78 (23) | |
| Ki67 | (Low) | 145 (37) | 138 (66) | 2.0 × 10⁻¹¹*** |
| | (High) | 246 (63) | 72 (34) | |
| HER2 | (No) | 680 (84) | 356 (92) | 0.0004*** |
| overexpression | (Yes) | 129 (16) | 33 (8) | |
| Triple negative | (No) | 569 (80) | 301 (91) | 1.6 × 10⁻⁶*** |
| | (Yes) | 146 (20) | 28 (9) | |

Epithelial mesenchymal phenotype

| | | | | |
|---|---|---|---|---|
| Basal like | (No) | 575 (84) | 305 (94) | 9.3 × 10⁻⁶*** |
| | (Yes) | 111 (16) | 20 (6) | |
| CK5/6 | (Negative) | 575 (81) | 287 (89) | 0.0035** |
| | (Positive) | 132 (19) | 37 (11) | |
| EGFR | (Negative) | 517 (79) | 266 (88) | 0.001*** |
| | (Positive) | 139 (21) | 38 (12) | |
| Vimentin | (Negative) | 461 (86) | 143 (94) | 0.010** |
| | (Positive) | 72 (14) | 9 (6) | |
| E-Cadherin | (Loss) | 171 (42) | 201 (34) | 0.016* |
| | (Positive) | 239 (58) | 387 (66) | |

DNA repair pathway

| | | | | |
|---|---|---|---|---|
| BRCA1 | (Loss) | 140 (24) | 19 (7) | 3.6 × 10⁻⁹*** |
| | (Positive) | 443 (76) | 250 (93) | |
| ATM | (Loss) | 149 (52) | 18 (17) | 5.9 × 10⁻¹⁰*** |
| | (Positive) | 137 (48) | 87 (83) | |
| XRCC1 | (Loss) | 155 (20) | 9 (3) | 8.6 × 10⁻¹⁵*** |
| | (High) | 617 (80) | 346 (97) | |

Cell cycle/apoptosis regulators

| | | | | |
|---|---|---|---|---|
| p53 | (Negative) | 532 (78) | 261 (87) | 4.5 × 10⁻⁴*** |
| | (Positive) | 153 (22) | 38 (13) | |
| BCL-2 | (Negative) | 293 (39) | 79 (22) | 4.6 × 10 ⁻⁸*** |
| | (Positive) | 459 (61) | 275 (78) | |
| TOP2A | (Negative) | 328 (52) | 87 (30) | 1.4 × 10⁻⁹*** |
| | (Positive) | 306 (48) | 200 (70) | |
| p16 | (Negative) | 502 (82) | 249 (94) | 7.3 × 10⁻⁶*** |
| | (Positive) | 110 (18) | 17 (6) | |

*P < 0.05;
**P < 0.01;
***P < 0.001;
^{#}grade as defined by Nottingham grading system;
BRCA1, breast cancer 1, early onset;
HER2; human epidermal growth factor receptor 2;
ER, estrogen receptor;
PR, progesterone receptor;
AR, androgen receptor;
CK, cytokeratin;
Basal like, ER−, HER2 and positive expression of either CK5/6, CK14 or EGFR;
Triple negative, ER−/PR−/HER2

TABLE 5
Multivariate analysis using Cox regression analysis confirms that SHON protein
expression is independent prognostic factor.

| | BCSS | | DFS | | DM-FS | |
|---|---|---|---|---|---|---|
| | HR | | HR | | HR | |
| Variable | (CI 95%) | p | (CI 95%) | p | (CI 95%) | p |
| SHON (-) | 1.4 | 0.017* | 1.3 | 0.02* | 1.3 | 0.037* |
| | (1.1-1.9) | | (1.1-1.7) | | (1.0-1.7) | |
| ER(-) | 1.2 | 0.06 | 1.1 | 0.5 | 1.1 | 0.6 |
| | (0.99-1.7) | | (0.9-1.4) | | (0.8-1.4) | |
| Tumour | 1.3 | 0.0004*** | 1.2 | 0.003** | 1.3 | 0.0003*** |
| size | (1.1-1.5) | | (1.1-1.7) | | (1.1-1.4) | |
| Grade | | | | | | |
| G1 | 1.0 | 4.9 × 10⁻⁷*** | 1.0 | 0.018* | 1.0 | 0.00001*** |
| G2 | 2.2 | | 1.2 | | 1.7 | |
| | (1.2-4.0) | | (0.9-1.9) | | (1.1-2.6) | |
| G3 | 3.8 | | 1.6 | | 2.6 | |
| | (2.2-6.7) | | (1.1-2.3) | | (1.7-4.0) | |
| Lymph node | | | | | | |
| Negative | 1 | 5.7 × 10⁻¹²** | 1 | 2.0 × 10⁻¹²** | 1 | 4 × 10⁻¹⁴*** |
| Positive | 1.5 | | 1.3 | | 1.5 | |
| (1-3 nodes) | (1.1-1.9) | | (1.1-1.7) | | (1.1-1.9) | |
| Positive | 3.9 | | 3.6 | | 4.2 | |
| (>3 nodes) | (2.7-5.7) | | (2.6-5.1) | | (2.9-6.0) | |

*P < 0.05;
***P < 0.001;
BCSS, breast cancer specific survival;
DFS, disease free survival;
DM-FS, distant metastases;
HR, hazard ratio;
CI, confident interval

### Example 3

### Production of Mouse SHON Monoclonal Antibodies

Mouse monoclonal SHON antibodies were produced from SHON antigen using 1) synthetic SHON peptides GGTTDLPHGP (SEQ ID NO: 33), PATAPISNQT (SEQ ID NO: 34), NQTLGVFPTQ (SEQ ID NO: 35) and PTQSITSHFQ (SEQ ID NO: 36) by Abmart (Shanghai) Co. Ltd; and 2) the purified GST-SHONa fusion protein by Biomedical Science institute, A*STAR, Singapore.

### Example 4

### Production and Specificity of Mouse Monoclonal Antibodies Against SHON

TABLE 6
Elisa screening of mouse monoclonal SHON antibodies using
synthetic SHON peptides (SEQ ID NOS 34, 33-36, 33, 34, 34, 34,
33, 35, 34, 35, 34, 34, 35, 34, 35 and 35, respectively, in
order of appearance).

| mAbNo. | Clone names | SHON peptides* | Elisa |
|---|---|---|---|
| 1# | 7897-1hz-2M12/2F11_120409 | PATAPISNQT | 128 |
| 2# | 7897-1hz-1M12/1L23 120414 | X | 128 |
| 3# | 7897-1hz-2M12/1A14 120410 | GGTTDLPHGP | 128 |
| 4# | 7897-1hz-1M12/2P18 120416 | X | 128 |
| 5# | 7897-1hz-1M12/2D1 120417 | X | 128 |
| 6# | 7897-1hz-2M12/2D7 120412 | PATAPISNQT | 128 |
| 7# | 7897-1hz-3M12/106 120417 | NQTLGVFPTQ | 128 |
| 8# | 7897-1hz-3M12/1L24 120415 | PTQSITSHFQ | 128 |
| 9# | 7897-1hz-2M12/1H18 120412 | GGTTDLPHGP | 128 |
| 10# | 7897-1hz-2M12/2C21 120415 | PATAPISNQT | 128 |
| 11# | 7897-1hz-2M12/2J8 120409 | PATAPISNQT | 128 |
| 12# | 7897-1hz-1M12/2E19 120416 | X | 128 |
| 13# | 7897-1hz-2M12/2G3 120412 | PATAPISNQT | 128 |
| 14# | 7897-1hz-2M12/1I18 120415 | GGTTDLPHGP | 128 |
| 15# | 7897-1hz-3M12/1B10 120416 | NQTLGVPPTQ | 128 |
| 16# | 7897-1hz-1M12/2A4-120420 | X | 128 |
| 17# | 7897-1hz-2M12/6A19 120415 | PATAPISNQT | 128 |
| 18# | 7897-1hz-3M12/106 120417 | NQTLGVFPTQ | 128 |
| 19# | 7897-1hz-1M12/1A22 120424 | X | 128 |
| 20# | 7897-1hz-3M12/2P17 120422 | PATAPISNQT | 128 |
| 21# | 7897-1hz-3M12/2M20 120124 | PATAPISNQT | 128 |
| 22# | 7897-1hz-1M12/2A4 120420 | X | 128 |
| 23# | 7897-1hz-1M12/2D1-120417 | X | 128 |
| 24# | 7897-1hz-3M12/106 120417 | NQTLGVFPTQ | 128 |
| 25# | 7897-1hz-3M12/7P9 120416 | X | 128 |
| 26# | 7897-1hz-2M12/7D5 120415 | PATAPISNQT | 128 |
| 27# | 7897-1hz-2M12/1A14 120410 | X | 128 |
| 28# | 7897-1hz-1M12/6K22 120420 | X | 128 |
| 29# | 7897-1hz-3M12/6N2 120416 | NQTLGVFPTQ | 128 |
| 30# | 7897-1hz-1M12/7C20 120420 | X | 128 |
| 31# | 7897-1hz-3M12/604 120416 | NQTLGVFPTQ | 128 |
| 32# | 7897-1hz-1M12/5N24 120420 | X | 128 |
| 33# | 7897-1hz-1M12/6B5 120420 | X | 128 |
| 34# | 7897-1hz-2M12/1118-120415 | X | 128 |

*X, to be determined.

34 mouse monoclonal SHON antibodies were produced from SHON antigen using synthetic SHON peptides GGTTDLPHGP (SEQ ID NO: 33), PATAPISNQT (SEQ ID NO: 34), NQTLGVFPTQ (SEQ ID NO: 35) and PTQSITSHFQ (SEQ ID NO: 36) by Abmart (Shanghai) Co. Ltd and their affinity to the antigens were determined by ELISA (Table 6).

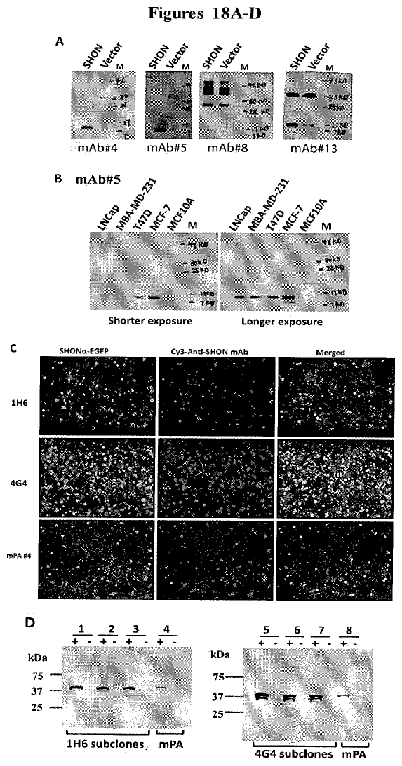

The specificity of 4 of them (SHON mAb#4, mAb#5, mAb#8 and mAb#13) was examined by western blot using MCF-7 cells stably transfected with the SHONa expression plasmid pIRESneo3-SHONα or the empty vector plasmid pIRESneo3 (FIG. 18A). All of them were able to detect both endogenous and forced SHON expression in the MCF-7 cells as a 12 kDa band. The larger bands at 24, 36, 48 kDa were either non-specific or multimers of SHON proteins.

The specificity of SHON mAb#5 was further tested by western blot using the human prostate cancer cell line LNCaP, breast cancer cell lines MBA-MD-231, T47D and MCF-7, and the normal breast cell line MCF10A. A single band of 12 kDa was detected in all the cell lines tested except in the MCF10A cell lines (FIG. 18B, left panel). The faint bands at 24 and 48 kDa observed after longer time of exposure were either non-specific or multimers of SHON proteins.

41 mouse monoclonal SHON antibodies were produced from SHON antigen using the purified GST-SHONa fusion protein by Biomedical Science institute, A*STAR, Singapore. The affinity of the antibodies to GST-SHONa or the GST tag was tested by ELISA (Table 7). The specificity of two of them (Clones 1H6 and 4G4) were examined by immunofluorescence staining using HEK293 cells transiently transfected with the expression plasmid pIRESneo3-SHONα-EGFP, which encodes SHONa with a C-terminal EGFP tag (FIG. 18C). The expression of SHONα was stained using the two antibodies and visualised with a Cy3 cyanine dye conjugated secondary antibody (Red). The expression of SHONα-EGFP was examined by fluorescence microscopy (Green). The co-localisation of green and red fluorescence staining in the pIRESneo3-SHONα-EGFP transfected cells demonstrated that the mouse anti-SHONα monoclonal antibodies specifically recognised SHON.

Three subclones of each of the two clones 1H6 and 4G4 were generated and their specificity was determined by Western blot analysis using HEK293 cells transiently transfected with the expression plasmid pIRESneo3-SHONα-EGFP, which encodes SHONα with a C-terminal EGFP tag, and the pEGFP-C1 empty vector, which encodes the EGFP protein (FIG. 18D). All the 6 subclones were able to specifically recognise the SHONα-EGFP fusion protein at the expected size of about 40 kDa, so did the mouse polyclonal SHON antibody from which the monoclonal antibodies were derived. The faint lower band was the result of protein degradation.

Therefore, we have produced mouse monoclonal SHON antibodies that were suitable for use in ELISA, Western blot and immunofluorescence staining.

TABLE 7
Elisa screening of mouse monoclonal SHON antibodies
using synthetic GST-SHONα fusion protein.

| No. | Clone | GST− | GST | Ratio |
|---|---|---|---|---|
| 1 | 1G6 | 0.6953 | 0.0804 | 8.64801 |
| 2 | 1H6 | 0.7382 | 0.0954 | 7.73795 |
| 3 | 1A9 | 0.2576 | 0.0485 | 5.31134 |
| 4 | 1G11 | 0.226 | 0.0366 | 6.17486 |
| 5 | 2D1 | 0.6445 | 0.0841 | 7.6635 |
| 6 | 2F2 | 0.4235 | 0.066 | 6.41667 |
| 7 | 2G2 | 0.5464 | 0.0583 | 9.37221 |
| 8 | 2E3 | 0.4301 | 0.0691 | 6.22431 |
| 9 | 2H3 | 0.4906 | 0.0759 | 6.46377 |
| 10 | 2A4 | 0.2905 | 0.0716 | 4.05726 |
| 11 | 2E6 | 0.2384 | 0.0564 | 4.22695 |
| 12 | 2E7 | 0.2896 | 0.0715 | 4.05035 |
| 13 | 2A8 | 0.3094 | 0.0561 | 5.51515 |
| 14 | 2D8 | 0.3957 | 0.0566 | 6.99117 |
| 15 | 2E8 | 0.2688 | 0.0503 | 5.34394 |
| 16 | 2H8 | 0.5621 | 0.0567 | 9.91358 |
| 17 | 2E9 | 0.4037 | 0.0696 | 5.80029 |
| 18 | 2B10 | 0.2633 | 0.0531 | 4.95857 |
| 19 | 2D11 | 0.2947 | 0.0735 | 4.00952 |
| 20 | 3H1 | 0.1693 | 0.0534 | 3.17041 |
| 21 | 3C4 | 0.3013 | 0.0771 | 3.90791 |
| 22 | 4G4 | 0.5894 | 0.071 | 8.36016 |
| 23 | 4D8 | 0.4959 | 0.0815 | 6.08466 |
| 24 | 6B1 | 0.217 | 0.06 | 3.61667 |
| 25 | 6G1 | 0.167 | 0.059 | 2.83051 |
| 26 | 6G2 | 0.192 | 0.057 | 3.36842 |
| 27 | 6G9 | 0.204 | 0.071 | 2.87324 |
| 28 | 7G1 | 0.189 | 0.062 | 3.04839 |
| 29 | 7H1 | 0.215 | 0.059 | 3.64407 |
| 30 | 7D2 | 0.212 | 0.056 | 3.78571 |
| 31 | 700 | 0.212 | 0.057 | 3.7193 |
| 32 | 7H3 | 0.376 | 0.084 | 4.47619 |
| 33 | 7C4 | 0.277 | 0.068 | 4.07353 |
| 34 | 7D5 | 0.255 | 0.063 | 4.04762 |
| 35 | 7H5 | 0.236 | 0.057 | 4.14035 |
| 36 | 7G7 | 0.2 | 0.057 | 3.50877 |
| 37 | 7E8 | 0.251 | 0.072 | 3.48611 |
| 38 | 7A11 | 0.232 | 0.05 | 4.64 |
| 39 | 8E5 | 0.189 | 0.051 | 3.70588 |
| 40 | 8A7 | 0.21 | 0.044 | 4.77273 |
| 41 | 8C8 | 0.194 | 0.057 | 3.40351 |

Thus preferred embodiments of the present invention have the advantage over the prior art of improved accuracy of predicting response of a tumour to endocrine therapy.

Aspects of the present invention have been described by way of example only and it should be appreciated that modifications and variations may be made without departing from the scope thereof as defined in the appended claims.

### REFERENCES

Abd El-Rehim D M et al. (2005). High-throughput protein express10n analysis using tissue microarray technology of a large well-characterised series identifies biologically distinct classes of breast cancer confirming recent cDNA expression analyses. Int J Cancer 116: 340-350.

Abdel-Fatah T M et al. (2010a). The biological, clinical and prognostic implications of p53 transcriptional pathways in breast cancers. J Pathol 220: 419-434.

Abdel-Fatah T M et al. (2010b). Proposal for a modified grading system based on mitotic index and Bc12 provides objective determination of clinical outcome for patients with breast cancer. J Pathol 222: 388-399.

Al S S et al. (2011). Signalling pathways involved in endocrine resistance in breast cancer and associations with epithelial to mesenchymal transition (Review). Int J Oncol 38: 1197-1217.

Baumgarten S C et al. (2012). Minireview: Inflammation: An Instigator of More Aggressive Estrogen Receptor (ER) Positive Breast Cancers. Mol Endocrinol.

Bean E S. (2000). Polyclonal Antibodies. In Howard Gary C. and Bathell Delia R. (Eds.), Basic Methods in Antibody Production and Characterization. CRC Press: Boca Raton, London, New York, pp. 31-49.

Bendtsen J D et al. (2004). Improved prediction of signal peptides: SignalP 3.0. J Mol Biol 340:783-795.

Callagy G M et al. (2006). Bcl-2 is a prognostic marker in breast cancer independently of the Nottingham Prognostic Index. Clin Cancer Res 12: 2468-2475.

Clark H F et al. (2003). The secreted protein discovery initiative (SPDI), a large-scale effort to identify novel human secreted and transmembrane proteins: a bioinformatics assessment. Genome Res 13: 2265-2270.

Clarke R et al. (2001). Cellular and molecular pharmacology of antiestrogen action and resistance. Pharmacol Rev 53: 25-71.

Clarke R et al. (2003). Antiestrogen resistance in breast cancer and the role of estrogen receptor signaling. Oncogene 22: 7316-7339.

Colombo P E et al. (2011). Microarrays in the 2010s: the contribution of microarray-based gene expression profiling to breast cancer classification, prognostication and prediction. Breast Cancer Res 13: 212.

Cosman F et al. (1999). Selective estrogen receptor modulators: clinical spectrum. Endocr Rev 20: 418-434.

Dowsett M et al. (2005). Biological characteristics of the pure antiestrogen fulvestrant: overcoming endocrine resistance. Breast Cancer Res Treat 93 Suppl 1: S11-S18.

Early Breast Cancer Trialists' Collaborative Group (EBCTCG). (2005). Effects of chemotherapy and hormonal therapy for early breast cancer on recurrence and 15-year survival: an overview of the randomised trials. Lancet 365: 1687-1717.

Elsheikh S et al. (2008). CCND1 amplification and cyclin DI expression in breast cancer and their relation with proteomic subgroups and patient outcome. Breast Cancer Res Treat 109: 325-335.

Elston C W et al. (1991). Pathological prognostic factors in breast cancer. I. The value of histological grade in breast cancer: experience from a large study with long-term follow-up. Histopathology 19: 403-410.

Encarnacion C A et al. (1993). Measurement of steroid hormone receptors in breast cancer patients on tamoxifen. Breast Cancer Res Treat 26: 237-246.

Fan C et al. (2006). Concordance among gene-expression-based predictors for breast cancer. N Engl J Med 355: 560-569.

Ferlay J et al. (2010). Estimates of worldwide burden of cancer in 2008: GLOBOCAN 2008. Int J Cancer.

Frohman M A et al. (1988). Rapid production of full-length cDNAs from rare transcripts: amplification using a single gene-specific oligonucleotide primer. Proc Natl Acad Sci USA 85: 8998-9002.

Geisler J et al. (2002). Influence of letrozole and anastrozole on total body aromatization and plasma estrogen levels in postmenopausal breast cancer patients evaluated in a randomized, cross-over study. J Clin Oncol 20: 751-757.

Giuliano M et al. (2011). Biological mechanisms and clinical implications of endocrine resistance in breast cancer. Breast 20 Suppl 3: S42-S49.

Green A R et al. (2008). The prognostic significance of steroid receptor co-regulators in breast cancer: co-repressor NCOR2/SMRT is an independent indicator of poor outcome. Breast Cancer Res Treat 110: 427-437.

Gutierrez M C et al. (2005). Molecular changes in tamoxifen-resistant breast cancer: relationship between estrogen receptor, HER-2, and p38 mitogen-activated protein kinase. J Clin Oneal 23: 2469-2476.

Haibe-Kains B et al. (2008). Comparison of prognostic gene expression signatures for breast cancer. BMC Genomics 9: 394.

Hayes D F et al. Why Hasn't Genomic Testing Changed the Landscape m Clinical Oncology. ASCO 2012 Educational Book: e52-e55. 2012.

Ref Type: Journal (Full)

Holm S. (1979). A simple sequentially rejective multiple test procedure. Scand J Stat 6: 65-70. Howard G C et al. (2001). Basic methods in antibody production and characterization. Karim-Jimenez Z et al. (2000). A dibasic motif involved in parathyroid honnone-induced down-regulation of the type Ila NaPi cotransporter. Proc Natl Acad Sci USA 97: 12896-12901.

Khatib A M et al. (2001). Inhibition of proprotein convertases is associated with loss of growth and tumorigenicity of HT-29 human colon carcinoma cells: importance of insulin-like growth factor-1 (IGF-1) receptor processing in IGF-1-mediated functions. J Biol Chem 276: 30686-30693.

Kok M et al. (2010). Gene expression profiles of the oestrogen receptor in breast cancer. Neth J Med 68: 291-302.

Ma C X et al. (2009). Predicting endocrine therapy responsiveness in breast cancer. Oncology (Williston Park) 23: 133-142.

McShane L M et al. (2005). Reporting recommendations for tumor marker prognostic studies (REMARK). J Natl Cancer Inst 97: 1180-1184.

Musgrove E A et al. (2009). Biological determinants of endocrine resistance in breast cancer. Nat Rev Cancer 9: 631-643.

Osborne C K et al. (2004). Fulvestrant: an oestrogen receptor antagonist with a novel mechanism of action. Br J Cancer 90 Suppl 1: S2-S6.

Paik S et al. (2004). A multigene assay to predict recurrence of tamoxifen-treated, node-negative breast cancer. N Engl J Med 351: 2817-2826.

Paik S et al. (2006). Gene expression and benefit of chemotherapy in women with node-negative, estrogen receptor-positive breast cancer. J Clin Oncol 24: 3726-3734.

Pailhoux E et al. (2001). A 11.7-kb deletion triggers intersexuality and polledness in goats. Nat Genet 29: 453-458.

Pinder S E et al. (1994). Pathological prognostic factors in breast cancer. III. Vascular invasion: relationship with recurrence and survival in a large study with long-term follow-up. Histopathology 24: 41-47.

Prat A et al. (2012). Concordance among gene expression-based predictors for ER-positive breast cancer treated with adjuvant tamoxifen. Ann Oncol.

Pritchard K. (2005). Endocrinology and hormone therapy in breast cancer: endocrine therapy in premenopausal women. Breast Cancer Res 7: 70-76.

Putti T C et al. (2005). Estrogen receptor-negative breast carcinomas: a review of morphology and immunophenotypical analysis. Mod Pathol 18: 26-35.

Rakha E A et al. (2005a). E-cadherin expression in invasive non-lobular carcinoma of the breast and its prognostic significance. Histopathology 46: 685-693.

Rakha E A et al. (2005b). Expression of mucins (MUCI, MUC2, MUC3, MUC4, MUC5AC and MUC6) and their prognostic significance in human breast cancer. Mod Pathol 18: 1295-1304.

Rakha E A et al. (2006a). Basal phenotype identifies a poor prognostic subgroup of breast cancer of clinical importance. Eur J Cancer 42: 3149-3156.

Rakha E A et al. (2007). Prognostic markers in triple-negative breast cancer. Cancer 109: 25-32.

Rakha E A et al. (2004). Expression of the transcription factor CTCF in invasive breast cancer: a candidate gene located at 1 6q22. 1. Br J Cancer 91: 1591-1596.

Rakha E A et al. (2006b). Morphological and immunophenotypic analysis of breast carcinomas with basal and myoepithelial differentiation. J Pathol 208: 495-506.

Riggins R B et al. (2005). Antiestrogens, aromatase inhibitors, and apoptosis in breast cancer. Vitam Horm 71: 201-237.

Rolland P et al. (2007). The p53 positive Bcl-2 negative phenotype is an independent marker of prognosis in breast cancer. Int J Cancer 120: 1311-1317.

Rosen P P. (2009). Rosen's breast pathology. Wolters Kluwer Health/Lippincott Williams & Wilkins: Philadelphia.

Sauter G et al. (2009). Guidelines for human epidermal growth factor receptor 2 testing: biologic and methodologic considerations. J Clin Oncol 27: 1323-1333.

Schook L B. (1987). Monoclonal antibody production techniques and applications. v. 33.

Sgroi D C. (2009). The HOXB13:IL17BR gene-expression ratio: a biomarker providing information above and beyond tumor grade. Biomark Med 3: 99-102.

Siegel D L. (2002). Recombinant monoclonal antibody technology. Tranifus Clin Biol 9: 15-22. Smale S T et al. (1989). The “initiator” as a transcription control element. Cell 57: 103-113.

Tan D S et al. (2008). Triple negative breast cancer: molecular profiling and prognostic impact in adjuvant anthracycline-treated patients. Breast Cancer Res Treat 111: 27-44.

Thomassen M et al. (2007). Comparison of gene sets for expression profiling: prediction of metastasis from low-malignant breast cancer. Clin Cancer Res 13: 5355-5360.

Turco E et al. (1990). The X boxes from promoters of HLA class II B genes at different loci do not complete for nuclear protein-specific binding. Immunogenetics 32: 117-128.

Welschof M et al. (2003). Generation and screening of a modular human scFv expression library from multiple donors. Methods Mol Biol 207: 103-121.

Wirapati P et al. (2008). Meta-analysis of gene expression profiles in breast cancer: toward a unified understanding of breast cancer subtyping and prognosis signatures. Breast Cancer Res 10: R65.

Yu J X et al. (2007). Pathway analysis of gene signatures predicting metastasis of node-negative primary breast cancer. BMC Cancer 7: 182.

Zwicker J et al. (1995). Cell cycle regulation of cdc25C transcription is mediated by the periodic repression of the glutamine-rich activators NF-Y and Spl. Nucleic Acids Res 23: 3822-3830.

1-23. (canceled)

24. A method of predicting the responsiveness to endocrine therapy of a cancer, comprising
identifying the presence of a SHON gene in a sample from a patient, wherein the SHON gene comprises a SHON polypeptide comprising an amino acid having substantial identity to SEQ ID NOS: 2, 5 or 6, or a SHON mRNA coding for a SHON polypeptide transcribed from a cDNA having substantial identity to SEQ ID NOS: 1, 3 or 4,
wherein the present of a SHON polypeptide or mRNA in the sample indicates that the cancer is endocrine therapy-responsive.

25. The method of claim 24, further comprising the step of identifying the presence of a estrogen receptor (ER) polypeptide or mRNA coding for the SHON polypeptide in the sample, wherein the presence of ER polypeptide or mRNA together with the presence of SHON polypeptide or mRNA indicates that the patient is endocrine therapy-responsive.

26. The method of claim 24, wherein the sample is a fluid, a tissue or a cell.

27. The method of claim 24, wherein the cancer comprises an estrogen receptor-positive cancer or a progesterone receptor-positive cancer.

28. The method of claim 24, wherein the cancer is breast cancer, colon cancer, prostate cancer, endometrial cancer, lung cancer, stomach cancer, liver cancer or ovarian cancer.

29. The method of claim 24, wherein the step of identifying the presence of SHON polypeptide in the sample comprises contacting the sample with a labelled antibody which specifically binds to the SHON polypeptide.

30. The method of claim 24, wherein the identification of the presence of a SHON mRNA coding for a SHON polypeptide in the sample comprises a nucleic acid amplification process for amplifying a fragment of RNA corresponding to the mRNA coding for the SHON polypeptide.

31. The method of claim 30, wherein the sample is contacted with a hybridization probe capable of specifically hybridizing to the mRNA coding for the SHON polypeptide.

32. The method of claim 24, wherein the presence of SHON polypeptide and/or SHON mRNA in the sample provides a prognosis of a prolonged disease-free survival of the cancer patient.

33. The method of claim 24, wherein the presence of SHON polypeptide and/or SHON mRNA in the sample provides prognosis of a prolonged distant metastasis-free survival of the cancer patient.

34. A kit for use in the method of claim 1, which comprises at least one antibody which specifically binds to the SHON polypeptide of SEQ ID NO. 6 in a sample from a patient to indicate whether a cancer is endocrine therapy-responsive.

35. A kit for use in the method of claim 1, which comprises at least one pair of complementary DNA primers which specifically bind to the SHON nucleotide sequence of SEQ ID NOS: 1, 3 or 4 in a sample from a patient to indicate whether a cancer is endocrine therapy-responsive.

36. A reagent which comprises at least one antibody which specifically binds to the SHON polypeptide of SEQ ID NO: 6, or at least one pair of complementary DNA primers which specifically bind to the SHON nucleotide sequence of SEQ ID NOS: 1, 3 or 4.

37. A method of treating a cancer, comprising
identifying the presence of a SHON gene in a sample from a patient, wherein the SHON gene comprises a SHON polypeptide comprising an amino acid having substantial identity to SEQ ID NOS: 2, 5 or 6, or a SHON mRNA coding for a SHON polypeptide transcribed from a cDNA having substantial identity to SEQ ID NOS: 1, 3 or 4, and wherein the present of a SHON polypeptide or mRNA in the sample indicates that the cancer is endocrine therapy-responsive;
administering to the patient having endocrine therapy-responsive cancer an effective amount of a selective estrogen receptor modulator.

38. The method of claim 37, wherein the selective estrogen receptor modulator comprises tamoxifen, raloxifene, or toremifene.

39. The method of claim 37, further comprising the step of identifying the presence of a estrogen receptor (ER) polypeptide or mRNA coding for the SHON polypeptide in the sample, wherein the presence of ER polypeptide or mRNA together with the presence of SHON polypeptide or mRNA indicates that the patient is endocrine therapy-responsive.

40. The method of claim 37, wherein the cancer is breast cancer, colon cancer, prostate cancer, endometrial cancer, lung cancer, stomach cancer, liver cancer or ovarian cancer.

41. The method of claim 37, wherein the step of identifying the presence of SHON polypeptide in the sample comprises contacting the sample with a labelled antibody which specifically binds to the SHON polypeptide.

42. The method of claim 37, wherein the identification of the presence of a SHON mRNA coding for a SHON polypeptide in the sample comprises a nucleic acid amplification process for amplifying a fragment of RNA corresponding to the mRNA coding for the SHON polypeptide.

43. The method of claim 37, wherein the sample is contacted with a hybridization probe capable of specifically hybridizing to the mRNA coding for the SHON polypeptide.
